SEQUENCE LISTING (508 sequences)

SEQ ID NO: 1 - DNA, 21 bp, Artificial Sequence
  Synthetic Construct
  misc_feature (20)..(21): deoxythymidine (dT) base
gcucuuuguccgcugaucutt

SEQ ID NO: 2 - DNA, 21 bp, Artificial Sequence
  Synthetic Construct
  misc_feature (20)..(21): deoxythymidine (dT) base
uguuggaccagaaguaaagtt

SEQ ID NO: 3 - DNA, 21 bp, Artificial Sequence
  Synthetic Construct
  misc_feature (20)..(21): deoxythymidine (dT) base
agguccuaugcauccucautt

SEQ ID NO: 4 - DNA, 21 bp, Artificial Sequence
  Synthetic Construct
  misc_feature (20)..(21): deoxythymidine (dT) base
cucuuuguccgcugaucuctt

SEQ ID NO: 5 - DNA, 21 bp, Artificial Sequence
  Synthetic Construct
  misc_feature (20)..(21): deoxythymidine (dT) base
aauuugccuuugaccuuuctt

SEQ ID NO: 6 - DNA, 21 bp, Artificial Sequence
  Synthetic Construct
  misc_feature (20)..(21): deoxythymidine (dT) base
auuugccuuugaccuuucutt

SEQ ID NO: 7 - DNA, 21 bp, Artificial Sequence
  Synthetic Construct
  misc_feature (20)..(21): deoxythymidine (dT) base
uuuccugcucuuuguccgctt

SEQ ID NO: 8 - DNA, 21 bp, Artificial Sequence
  Synthetic Construct
  misc_feature (20)..(21): deoxythymidine (dT) base
uucuucucagcccaggucctt

SEQ ID NO: 9 - DNA, 21 bp, Artificial Sequence
  Synthetic Construct
  misc_feature (20)..(21): deoxythymidine (dT) base
ucugcuuuuccugcucuuutt

SEQ ID NO: 10 - DNA, 21 bp, Artificial Sequence
  Synthetic Construct
  misc_feature (20)..(21): deoxythymidine (dT) base
uucugcuuuuccugcucuutt

SEQ ID NO: 11 - DNA, 21 bp, Artificial Sequence
  Synthetic Construct
  misc_feature (20)..(21): deoxythymidine (dT) base
aagguuccgaggggccauutt

SEQ ID NO: 12 - DNA, 21 bp, Artificial Sequence
  Synthetic Construct
  misc_feature (20)..(21): deoxythymidine (dT) base
aaaaggacaccguuucaggtt

SEQ ID NO: 13 - DNA, 21 bp, Artificial Sequence
  Synthetic Construct
  misc_feature (20)..(21): deoxythymidine (dT) base
aaaggacaccguuucagggtt

SEQ ID NO: 14 - DNA, 21 bp, Artificial Sequence
  Synthetic Construct
  misc_feature (20)..(21): deoxythymidine (dT) base
auguuggaccagaaguaaatt

SEQ ID NO: 15 - DNA, 21 bp, Artificial Sequence
  Synthetic Construct
  misc_feature (20)..(21): deoxythymidine (dT) base
ucuucucagcccagguccutt

SEQ ID NO: 16 - DNA, 21 bp, Artificial Sequence
  Synthetic Construct
  misc_feature (20)..(21): deoxythymidine (dT) base
ugucucaauuugccuuugatt

SEQ ID NO: 17 - DNA, 21 bp, Artificial Sequence
  Synthetic Construct
  misc_feature (20)..(21): deoxythymidine (dT) base
ccagguccuaugcauccuctt

SEQ ID NO: 18 - DNA, 21 bp, Artificial Sequence
  Synthetic Construct
  misc_feature (20)..(21): deoxythymidine (dT) base
cugcuuuuccugcucuuugtt

SEQ ID NO: 19 - DNA, 21 bp, Artificial Sequence
  Synthetic Construct
  misc_feature (20)..(21): deoxythymidine (dT) base
uucuuagggacuuguuuuctt

SEQ ID NO: 20 - DNA, 21 bp, Artificial Sequence
  Synthetic Construct
  misc_feature (20)..(21): deoxythymidine (dT) base
aucuaugcagggcuguuautt

SEQ ID NO: 21 - DNA, 21 bp, Artificial Sequence
  Synthetic Construct
  misc_feature (20)..(21): deoxythymidine (dT) base
uucuugucucaauuugccutt

SEQ ID NO: 22 - DNA, 21 bp, Artificial Sequence
  Synthetic Construct
  misc_feature (20)..(21): deoxythymidine (dT) base
gaaagcucaaagcuauguutt

SEQ ID NO: 23 - DNA, 21 bp, Artificial Sequence
  Synthetic Construct
  misc_feature (20)..(21): deoxythymidine (dT) base
uugaaagcucaaagcuaugtt

SEQ ID NO: 24 - DNA, 21 bp, Artificial Sequence
  Synthetic Construct
  misc_feature (20)..(21): deoxythymidine (dT) base
gguuugcacccggucuucutt

SEQ ID NO: 25 - DNA, 21 bp, Artificial Sequence
  Synthetic Construct
  misc_feature (20)..(21): deoxythymidine (dT) base
accagaaguaaaguguucutt

SEQ ID NO: 26 - DNA, 21 bp, Artificial Sequence
  Synthetic Construct
  misc_feature (20)..(21): deoxythymidine (dT) base
guuugcacccggucuucuutt

SEQ ID NO: 27 - DNA, 21 bp, Artificial Sequence
  Synthetic Construct
  misc_feature (20)..(21): deoxythymidine (dT) base
gucucaauuugccuuugactt

SEQ ID NO: 28 - DNA, 21 bp, Artificial Sequence
  Synthetic Construct
  misc_feature (20)..(21): deoxythymidine (dT) base
aggugccuucuugucucaatt

SEQ ID NO: 29 - DNA, 21 bp, Artificial Sequence
  Synthetic Construct
  misc_feature (20)..(21): deoxythymidine (dT) base
uugaggaaacuccucccuutt

SEQ ID NO: 30 - DNA, 21 bp, Artificial Sequence
  Synthetic Construct
  misc_feature (20)..(21): deoxythymidine (dT) base
uccuaugcauccucaucuatt

SEQ ID NO: 31 - DNA, 21 bp, Artificial Sequence
  Synthetic Construct
  misc_feature (20)..(21): deoxythymidine (dT) base
ugaaagcucaaagcuaugutt

SEQ ID NO: 32 - DNA, 21 bp, Artificial Sequence
  Synthetic Construct
  misc_feature (20)..(21): deoxythymidine (dT) base
uguuuucugcuuuuccugctt

SEQ ID NO: 33 - DNA, 21 bp, Artificial Sequence
  Synthetic Construct
  misc_feature (20)..(21): deoxythymidine (dT) base
cucuucuuugaaagcucaatt

SEQ ID NO: 34 - DNA, 21 bp, Artificial Sequence
  Synthetic Construct
  misc_feature (20)..(21): deoxythymidine (dT) base
aaagguuccgaggggccautt

SEQ ID NO: 35 - DNA, 21 bp, Artificial Sequence
  Synthetic Construct
  misc_feature (20)..(21): deoxythymidine (dT) base
cuucuugucucaauuugcctt

SEQ ID NO: 36 - DNA, 21 bp, Artificial Sequence
  Synthetic Construct
  misc_feature (20)..(21): deoxythymidine (dT) base
auugaggaaacuccucccutt

SEQ ID NO: 37 - DNA, 21 bp, Artificial Sequence
  Synthetic Construct
  misc_feature (20)..(21): deoxythymidine (dT) base
uauguuggaccagaaguaatt

SEQ ID NO: 38 - DNA, 21 bp, Artificial Sequence
  Synthetic Construct
  misc_feature (20)..(21): deoxythymidine (dT) base
caggugccuucuugucucatt

SEQ ID NO: 39 - DNA, 21 bp, Artificial Sequence
  Synthetic Construct
  misc_feature (20)..(21): deoxythymidine (dT) base
caauuugccuuugaccuuutt

SEQ ID NO: 40 - DNA, 21 bp, Artificial Sequence
  Synthetic Construct
  misc_feature (20)..(21): deoxythymidine (dT) base
guaaaguguucucguuucutt

SEQ ID NO: 41 - DNA, 21 bp, Artificial Sequence
  Synthetic Construct
  misc_feature (20)..(21): deoxythymidine (dT) base
cccagguccuaugcauccutt

SEQ ID NO: 42 - DNA, 21 bp, Artificial Sequence
  Synthetic Construct
  misc_feature (20)..(21): deoxythymidine (dT) base
ccuaugcauccucaucuautt

SEQ ID NO: 43 - DNA, 21 bp, Artificial Sequence
  Synthetic Construct
  misc_feature (20)..(21): deoxythymidine (dT) base
aagcuuccgaaaaggacactt

SEQ ID NO: 44 - DNA, 21 bp, Artificial Sequence
  Synthetic Construct
  misc_feature (20)..(21): deoxythymidine (dT) base
uugcacccggucuucuugctt

SEQ ID NO: 45 - DNA, 21 bp, Artificial Sequence
  Synthetic Construct
  misc_feature (20)..(21): deoxythymidine (dT) base
guccgcugaucuccugggatt

SEQ ID NO: 46 - DNA, 21 bp, Artificial Sequence
  Synthetic Construct
  misc_feature (20)..(21): deoxythymidine (dT) base
uugaaagguuccgaggggctt

SEQ ID NO: 47 - DNA, 21 bp, Artificial Sequence
  Synthetic Construct
  misc_feature (20)..(21): deoxythymine (dT) base
cauccucaucuaugcagggtt

SEQ ID NO: 48 - DNA, 21 bp, Artificial Sequence
  Synthetic Construct
  misc_feature (20)..(21): deoxythymine (dT) base
ggugccuucuugucucaautt

SEQ ID NO: 49 - DNA, 21 bp, Artificial Sequence
  Synthetic Construct
  misc_feature (20)..(21): deoxythymine (dT) base
ugccuuugaccuuucuuagtt

SEQ ID NO: 50 - DNA, 21 bp, Artificial Sequence
  Synthetic Construct
  misc_feature (20)..(21): deoxythymine (dT) base
cucaaagcuauguuggacctt

SEQ ID NO: 51 - DNA, 21 bp, Artificial Sequence
  Synthetic Construct
  misc_feature (20)..(21): deoxythymine (dT) base
gaccagaaguaaaguguuctt

SEQ ID NO: 52 - DNA, 21 bp, Artificial Sequence
  Synthetic Construct
  misc_feature (20)..(21): deoxythymine (dT) base
cugaucuccugggaagaaatt

SEQ ID NO: 53 - DNA, 21 bp, Artificial Sequence
  Synthetic Construct
  misc_feature (20)..(21): deoxythymine (dT) base
uuccugcucuuuguccgcutt

SEQ ID NO: 54 - DNA, 21 bp, Artificial Sequence
  Synthetic Construct
  misc_feature (20)..(21): deoxythymine (dT) base
cagguccuaugcauccucatt

SEQ ID NO: 55 - DNA, 21 bp, Artificial Sequence
  Synthetic Construct
  misc_feature (20)..(21): Synthetic Construct
uccgcugaucuccugggaatt

SEQ ID NO: 56 - DNA, 21 bp, Artificial Sequence
  Synthetic Construct
  misc_feature (20)..(21): deoxythymine (dT) base
uagcaucgagugguauuuutt

SEQ ID NO: 57 - DNA, 21 bp, Artificial Sequence
  Synthetic Construct
  misc_feature (20)..(21): deoxythymine (dT) base
gaucuccugggaagaaagctt

SEQ ID NO: 58 - DNA, 21 bp, Artificial Sequence
  Synthetic Construct
  misc_feature (20)..(21): deoxythymine (dT) base
ucaauuugccuuugaccuutt

SEQ ID NO: 59 - DNA, 21 bp, Artificial Sequence
  Synthetic Construct
  misc_feature (20)..(21): deoxythymine (dT) base
aagcuauguuggaccagaatt

SEQ ID NO: 60 - DNA, 21 bp, Artificial Sequence
  Synthetic Construct
  misc_feature (20)..(21): deoxythymine (dT) base
uugccuuugaccuuucuuatt

SEQ ID NO: 61 - DNA, 21 bp, Artificial Sequence
  Synthetic Construct
  misc_feature (20)..(21): deoxythymine (dT) base
uugaccuuucuuagggacutt

SEQ ID NO: 62 - DNA, 21 bp, Artificial Sequence
  Synthetic Construct
  misc_feature (20)..(21): deoxythymine (dT) base
ugauagucccguuuucuuctt

SEQ ID NO: 63 - DNA, 21 bp, Artificial Sequence
  deoxythymine (dT) base
  misc_feature (20)..(21): deoxythymine (dT) base
caucuaugcagggcuguuatt

SEQ ID NO: 64 - DNA, 21 bp, Artificial Sequence
  Synthetic Construct
  misc_feature (20)..(21): deoxythymine (dT) base
ucucuucuuugaaagcucatt

SEQ ID NO: 65 - DNA, 21 bp, Artificial Sequence
  Synthetic Construct
  misc_feature (20)..(21): deoxythymine (dT) base
agcuauguuggaccagaagtt

SEQ ID NO: 66 - DNA, 21 bp, Artificial Sequence
  Synthetic Construct
  misc_feature (20)..(21): deoxythymine (dT) base
uuucugcuuuuccugcucutt

SEQ ID NO: 67 - DNA, 21 bp, Artificial Sequence
  Synthetic Construct
  misc_feature (20)..(21): deoxythymine (dT) base
uuuucugcuuuuccugcuctt

SEQ ID NO: 68 - DNA, 21 bp, Artificial Sequence
  Synthetic Construct
  misc_feature (20)..(21): deoxythymine (dT) base
cgcugaucuccugggaagatt

SEQ ID NO: 69 - DNA, 21 bp, Artificial Sequence
  Synthetic Construct
  misc_feature (20)..(21): deoxythymine (dT) base
gcuauguuggaccagaagutt

SEQ ID NO: 70 - DNA, 21 bp, Artificial Sequence
  Synthetic Construct
  misc_feature (20)..(21): deoxythymine (dT) base
uguagcaucgagugguauutt

SEQ ID NO: 71 - DNA, 21 bp, Artificial Sequence
  Synthetic Construct
  misc_feature (20)..(21): deoythymine (dT) base
cgaaaaggacaccguuucatt

SEQ ID NO: 72 - DNA, 21 bp, Artificial Sequence
  Synthetic Construct
  misc_feature (20)..(21): deoxythymine (dT) base
agcuuccgaaaaggacacctt

SEQ ID NO: 73 - DNA, 21 bp, Artificial Sequence
  Synthetic Construct
  misc_feature (20)..(21): deoxythymine (dT) base
ccugggaagaaagcuuccgtt

SEQ ID NO: 74 - DNA, 21 bp, Artificial Sequence
  Synthetic Construct
  misc_feature (20)..(21): deoxythymine (dT) base
uuuuccugcucuuuguccgtt

SEQ ID NO: 75 - DNA, 21 bp, Artificial Sequence
  Synthetic Construct
  misc_feature (20)..(21): deoxythymine (dT) base
ugcucuuuguccgcugauctt

SEQ ID NO: 76 - DNA, 21 bp, Artificial Sequence
  Synthetic Construct
  misc_feature (20)..(21): deoxythymine (dT) base
uuggaccagaaguaaagugtt

SEQ ID NO: 77 - DNA, 21 bp, Artificial Sequence
  Synthetic Construct
  misc_feature (20)..(21): deoxythymine (dT) base
cuuuguccgcugaucuccutt

SEQ ID NO: 78 - DNA, 21 bp, Artificial Sequence
  Synthetic Construct
  misc_feature (20)..(21): deoxythymine (dT) base
cuugucucaauuugccuuutt

SEQ ID NO: 79 - DNA, 21 bp, Artificial Sequence
  Synthetic Construct
  misc_feature (20)..(21): deoxythymine (dT) base
cacccggucuucuugcccutt

SEQ ID NO: 80 - DNA, 21 bp, Artificial Sequence
  Synthetic Construct
  misc_feature (20)..(21): deoxythymine (dT) base
cuuugaccuuucuuagggatt

SEQ ID NO: 81 - DNA, 21 bp, Artificial Sequence
  Synthetic Construct
  misc_feature (20)..(21): deoxythymine (dT) base
ccuuugaccuuucuuagggtt

SEQ ID NO: 82 - DNA, 21 bp, Artificial Sequence
  Synthetic Construct
  misc_feature (20)..(21): deoxythymine (dT) base
ugggaagaaagcuuccgaatt

SEQ ID NO: 83 - DNA, 21 bp, Artificial Sequence
  Synthetic Construct
  misc_feature (20)..(21): deoxythymine (dT) base
ucuaugcagggcuguuauctt

SEQ ID NO: 84 - DNA, 21 bp, Artificial Sequence
  Synthetic Construct
  misc_feature (20)..(21): deoxythymine (dT) base
aaagcuauguuggaccagatt

SEQ ID NO: 85 - DNA, 21 bp, Artificial Sequence
  Synthetic Construct
  misc_feature (20)..(21): deoxythymine (dT) base
ucucaauuugccuuugacctt

SEQ ID NO: 86 - DNA, 21 bp, Artificial Sequence
  Synthetic Construct
  misc_feature (20)..(21): deoxythymine (dT) base
ugcauccucaucuaugcagtt

SEQ ID NO: 87 - DNA, 21 bp, Artificial Sequence
  Synthetic Construct
  misc_feature (20)..(21): deoxythymine (dT) base
gaaagguuccgaggggccatt

SEQ ID NO: 88 - DNA, 21 bp, Artificial Sequence
  Synthetic Construct
  misc_feature (20)..(21): deoxythymine (dT) base
gccuucuugucucaauuugtt

SEQ ID NO: 89 - DNA, 21 bp, Artificial Sequence
  Synthetic Construct
  misc_feature (20)..(21): deoxythymine (dT) base
acuccucccuuuuaauauctt

SEQ ID NO: 90 - DNA, 21 bp, Artificial Sequence
  Synthetic Construct
  misc_feature (20)..(21): deoxythymine (dT) base
gaagaaagcuuccgaaaagtt

SEQ ID NO: 91 - DNA, 21 bp, Artificial Sequence
  Synthetic Construct
  misc_feature (20)..(21): deoxythymine (dT) base
ugaccuuucuuagggacuutt

SEQ ID NO: 92 - DNA, 21 bp, Artificial Sequence
  Synthetic Construct
  misc_feature (20)..(21): deoxythymine (dT) base
aagcucaaagcuauguuggtt

SEQ ID NO: 93 - DNA, 21 bp, Artificial Sequence
  Synthetic Construct
  misc_feature (20)..(21): deoxythymine (dT) base
caguuccggguuugcaccctt

SEQ ID NO: 94 - DNA, 21 bp, Artificial Sequence
  Synthetic Construct
  misc_feature (20)..(21): deoxythymine (dT) base
ugcacccggucuucuugcctt

SEQ ID NO: 95 - DNA, 21 bp, Artificial Sequence
  Synthetic Construct
  misc_feature (20)..(21): deoxythymine (dT) base
ccuccuggguuuaacgcautt

SEQ ID NO: 96 - DNA, 21 bp, Artificial Sequence
  Synthetic Construct
  misc_feature (20)..(21): deoxythymine (dT) base
gcuuccgaaaaggacaccgtt

SEQ ID NO: 97 - DNA, 21 bp, Artificial Sequence
  Synthetic Construct
  misc_feature (20)..(21): deoxythymine (dT) base
cucaauuugccuuugaccutt

SEQ ID NO: 98 - DNA, 21 bp, Artificial Sequence
  Synthetic Construct
  misc_feature (20)..(21): deoxythymine (dT) base
gguccuaugcauccucauctt

SEQ ID NO: 99 - DNA, 21 bp, Artificial Sequence
  Synthetic Construct
  misc_feature (20)..(21): deoxythymine (dT) base
guuuucugcuuuuccugcutt

SEQ ID NO: 100 - DNA, 21 bp, Artificial Sequence
  Synthetic Construct
  misc_feature (20)..(21): deoxythymine (dT) base
uuguuuucugcuuuuccugtt

SEQ ID NO: 101 - DNA, 21 bp, Artificial Sequence
  Synthetic Construct
  misc_feature (20)..(21): deoxythymine (dT) base
cagaaguaaaguguucucgtt

SEQ ID NO: 102 - DNA, 21 bp, Artificial Sequence
  Synthetic Construct
  misc_feature (20)..(21): deoxythymine (dT) base
ugcuuuuccugcucuuugutt

SEQ ID NO: 103 - DNA, 21 bp, Artificial Sequence
  Synthetic Construct
  misc_feature (20)..(21): deoxythymine (dT) base
gcugaucuccugggaagaatt

SEQ ID NO: 104 - DNA, 21 bp, Artificial Sequence
  Synthetic Construct
  misc_feature (20)..(21): deoxythymine (dT) base
acaccguuucaggggcgagtt

SEQ ID NO: 105 - DNA, 21 bp, Artificial Sequence
  Artificial Sequence
  misc_feature (20)..(21): deoxythymine (dT) base
gaaaaggacaccguuucagtt

SEQ ID NO: 106 - DNA, 21 bp, Artificial Sequence
  Synthtetic Construct
  misc_feature (20)..(21): deoxythymine (dT) base
uuucuucucagcccagguctt

SEQ ID NO: 107 - DNA, 21 bp, Artificial Sequence
  Synthetic Construct
  misc_feature (20)..(21): deoxythymine (dT) base
ucaucuaugcagggcuguutt

SEQ ID NO: 108 - DNA, 21 bp, Artificial Sequence
  Synthetic Construct
  misc_feature (20)..(21): deoxythymine (dT) base
ugaggaaacuccucccuuutt

SEQ ID NO: 109 - DNA, 21 bp, Artificial Sequence
  Synthetic Construct
  misc_feature (20)..(21): deoxythymine (dT) base
aggggccauugaggaaacutt

SEQ ID NO: 110 - DNA, 21 bp, Artificial Sequence
  Synthetic Construct
  misc_feature (20)..(21): deoxythymine (dT) base
uuugccuuugaccuuucuutt

SEQ ID NO: 111 - DNA, 21 bp, Artificial Sequence
  Synthetic Construct
  misc_feature (20)..(21): deoxythymine (dT) base
uuguccgcugaucuccuggtt

SEQ ID NO: 112 - DNA, 21 bp, Artificial Sequence
  Synthetic Construct
  misc_feature (20)..(21): deoxythymine (dT) base
uucucagcccagguccuautt

SEQ ID NO: 113 - DNA, 21 bp, Artificial Sequence
  Synthetic Construct
  misc_feature (20)..(21): deoxythymine (dT) base
gugccuucuugucucaauutt

SEQ ID NO: 114 - DNA, 21 bp, Artificial Sequence
  Synthetic Construct
  misc_feature (20)..(21): deoxythymidine (dT) base
ggguuuaacgcauugugautt

SEQ ID NO: 115 - DNA, 21 bp, Artificial Sequence
  Synthetic Construct
  misc_feature (20)..(21): deoxythymine (dT) base
uggaccagaaguaaagugutt

SEQ ID NO: 116 - DNA, 21 bp, Artificial Sequence
  Synthetic Construct
  misc_feature (20)..(21): deoxythymine (dT) base
uugucucaauuugccuuugtt

SEQ ID NO: 117 - DNA, 21 bp, Artificial Sequence
  Synthetic Construct
  misc_feature (20)..(21): deoxythymine (dT) base
gggaagaaagcuuccgaaatt

SEQ ID NO: 118 - DNA, 21 bp, Artificial Sequence
  Synthetic Construct
  misc_feature (20)..(21): deoxythymine (dT) base
gcccagguccuaugcaucctt

SEQ ID NO: 119 - DNA, 21 bp, Artificial Sequence
  Synthetic Construct
  misc_feature (20)..(21): deoxythymine (dT) base
uuugcacccggucuucuugtt

SEQ ID NO: 120 - DNA, 21 bp, Artificial Sequence
  Synthetic Construct
  misc_feature (20)..(21): Synthetic Construct
cugggaagaaagcuuccgatt

SEQ ID NO: 121 - DNA, 21 bp, Artificial Sequence
  Synthetic Construct
  misc_feature (20)..(21): Synthetic Construct
ccaguuccggguuugcacctt

SEQ ID NO: 122 - DNA, 21 bp, Artificial Sequence
  Synthetic Construct
  misc_feature (20)..(21): deoxythymine (dT) base
guuuucuucucagcccaggtt

SEQ ID NO: 123 - DNA, 21 bp, Artificial Sequence
  Synthetic Construct
  misc_feature (20)..(21): deoxythymine (dT) base
ucuugucucaauuugccuutt

SEQ ID NO: 124 - DNA, 21 bp, Artificial Sequence
  Synthetic Construct
  misc_feature (20)..(21): deoxythymine (dT) base
guccuaugcauccucaucutt

SEQ ID NO: 125 - DNA, 21 bp, Artificial Sequence
  Synthetic Construct
  misc_feature (20)..(21): deoxythymine (dT) base
uuccgaaaaggacaccguutt

SEQ ID NO: 126 - DNA, 21 bp, Artificial Sequence
  Synthetic Construct
  misc_feature (20)..(21): deoxythymine (dT) base
uuuucuucucagcccaggutt

SEQ ID NO: 127 - DNA, 21 bp, Artificial Sequence
  Synthetic Construct
  misc_feature (20)..(21): deoxythymine (dT) base
gcauugugauagucccguutt

SEQ ID NO: 128 - DNA, 21 bp, Artificial Sequence
  Synthetic Construct
  misc_feature (20)..(21): deoxythymine (dT) base
acuugaaagguuccgagggtt

SEQ ID NO: 129 - DNA, 21 bp, Artificial Sequence
  Synthetic Construct
  misc_feature (20)..(21): deoxythymine (dT) base
gguuccgaggggccauugatt

SEQ ID NO: 130 - DNA, 21 bp, Artificial Sequence
  Synthetic Construct
  misc_feature (20)..(21): deoxythymine (dT) base
ccuucuugucucaauuugctt

SEQ ID NO: 131 - DNA, 21 bp, Artificial Sequence
  Synthetic Construct
  misc_feature (20)..(21): deoxythymine (dT) base
uggguuuaacgcauugugatt

SEQ ID NO: 132 - DNA, 21 bp, Artificial Sequence
  Synthetic Construct
  misc_feature (20)..(21): deoxythymine (dT) base
uuugaccuuucuuagggactt

SEQ ID NO: 133 - DNA, 21 bp, Artificial Sequence
  Synthetic Construct
  misc_feature (20)..(21): deoxythymine (dT) base
gugauagucccguuuucuutt

SEQ ID NO: 134 - DNA, 21 bp, Artificial Sequence
  Synthetic Construct
  misc_feature (20)..(21): deoxythymine (dT) base
uacaggugccuucuugucutt

SEQ ID NO: 135 - DNA, 21 bp, Artificial Sequence
  deoxythymine (dT) base
ugugauagucccguuuucutt

SEQ ID NO: 136 - DNA, 21 bp, Artificial Sequence
  Synthetic Construct
  misc_feature (20)..(21): deoxythymine (dT) base
cagggcuguuaucugcauatt

SEQ ID NO: 137 - DNA, 21 bp, Artificial Sequence
  Synthetic Construct
  misc_feature (20)..(21): deoxythymine (dT) base
ccguuucaggggcgagugatt

SEQ ID NO: 138 - DNA, 21 bp, Artificial Sequence
  Synthetic Construct
  misc_feature (20)..(21): deoxythymine (dT) base
gccauugaggaaacuccuctt

SEQ ID NO: 139 - DNA, 21 bp, Artificial Sequence
  Synthetic Construct
  misc_feature (20)..(21): deoxythymine (dT) base
ccauugaggaaacuccucctt

SEQ ID NO: 140 - DNA, 21 bp, Artificial Sequence
  Synthetic Construct
  misc_feature (20)..(21): deoxythymine (dT) base
cucagcccagguccuaugctt

SEQ ID NO: 141 - DNA, 21 bp, Artificial Sequence
  Synthetic Construct
  misc_feature (20)..(21): Synthetic Construct
  misc_feature (20)..(21): deoxythymine (dT) base
ugaucuccugggaagaaagtt

SEQ ID NO: 142 - DNA, 21 bp, Artificial Sequence
  Synthetic Construct
  misc_feature (20)..(21): deoxythymine (dT) base
aauguagcaucgagugguatt

SEQ ID NO: 143 - DNA, 21 bp, Artificial Sequence
  Synthetic Construct
  misc_feature (20)..(21): deoxythymine (dT) base
cuucuuugaaagcucaaagtt

SEQ ID NO: 144 - DNA, 21 bp, Artificial Sequence
  Synthetic Construct
  misc_feature (20)..(21): deoxythymine (dT) base
aaggacaccguuucaggggtt

SEQ ID NO: 145 - DNA, 21 bp, Artificial Sequence
  Synthetic Construct
  misc_feature (20)..(21): deoxythymine (dT) base
ccgaaaaggacaccguuuctt

SEQ ID NO: 146 - DNA, 21 bp, Artificial Sequence
  Synthetic Construct
  misc_feature (20)..(21): deoxythymine (dT) base
aacuugaaagguuccgaggtt

SEQ ID NO: 147 - DNA, 21 bp, Artificial Sequence
  deoxythymine (dT) base
ccagaaguaaaguguucuctt

SEQ ID NO: 148 - DNA, 21 bp, Artificial Sequence
  Synthetic Construct
  misc_feature (20)..(21): deoxythymine (dT) base
gucccguuuucuucucagctt

SEQ ID NO: 149 - DNA, 21 bp, Artificial Sequence
  Synthetic Construct
  misc_feature (20)..(21): deoxythymine (dT) base
ccucaucuaugcagggcugtt

SEQ ID NO: 150 - DNA, 21 bp, Artificial Sequence
  Synthetic Construct
  misc_feature (20)..(21): deoxythymine (dT) base
gaaagcuuccgaaaaggactt

SEQ ID NO: 151 - DNA, 21 bp, Artificial Sequence
  Synthetic Construct
  misc_feature (20)..(21): deoxythymine (dT) base
uccugcucuuuguccgcugtt

SEQ ID NO: 152 - DNA, 21 bp, Artificial Sequence
  Synthetic Construct
  misc_feature (20)..(21): deoxythymine (dT) base
ccuuucuuagggacuuguutt

SEQ ID NO: 153 - DNA, 21 bp, Artificial Sequence
  deoxythymine (dT) base
  misc_feature (20)..(21): deoxythymine (dT) base
cuuagggacuuguuuucugtt

SEQ ID NO: 154 - DNA, 21 bp, Artificial Sequence
  Synthetic Construct
  misc_feature (20)..(21): deoxythymine (dT) base
aaaaccuccuggguuuaactt

SEQ ID NO: 155 - DNA, 21 bp, Artificial Sequence
  Synthetic Construct
  misc_feature (20)..(21): Synthetic Construct
  misc_feature (20)..(21): deoxythymine (dT) base
aggacaccguuucaggggctt

SEQ ID NO: 156 - DNA, 21 bp, Artificial Sequence
  Synthetic Construct
  misc_feature (20)..(21): Synthetic Construct
agaaagcuuccgaaaaggatt

SEQ ID NO: 157 - DNA, 21 bp, Artificial Sequence
  Synthetic Construct
  misc_feature (20)..(21): deoxythymine (dT) base
aggaaacuccucccuuuuatt

SEQ ID NO: 158 - DNA, 21 bp, Artificial Sequence
  deoxythymine (dT) base
cgaggggccauugaggaaatt

SEQ ID NO: 159 - DNA, 21 bp, Artificial Sequence
  Synthetic Construct
  misc_feature (20)..(21): deoxythymine (dT) base
ccuggguuuaacgcauugutt

SEQ ID NO: 160 - DNA, 21 bp, Artificial Sequence
  Synthetic Construct
  misc_feature (20)..(21): deoxythymine (dT) base
cuuugaaagcucaaagcuatt

SEQ ID NO: 161 - DNA, 21 bp, Artificial Sequence
  Synthetic Construct
  misc_feature (20)..(21): deoxythymine (dT) base
agaaguaaaguguucucgutt

SEQ ID NO: 162 - DNA, 21 bp, Artificial Sequence
  Synthetic Construct
  misc_feature (20)..(21): deoxythymine (dT) base
augcauccucaucuaugcatt

SEQ ID NO: 163 - DNA, 21 bp, Artificial Sequence
  Synthetic Construct
  misc_feature (20)..(21): deoxythymine (dT) base
cuugaaagguuccgaggggtt

SEQ ID NO: 164 - DNA, 21 bp, Artificial Sequence
  Synthetic Construct
  misc_feature (20)..(21): deoxythymine (dT) base
gcuuuuccugcucuuuguctt

SEQ ID NO: 165 - DNA, 21 bp, Artificial Sequence
  Synthetic Construct
  misc_feature (20)..(21): deoxythymine (dT) base
agaucagcggacaaagagctt

SEQ ID NO: 166 - DNA, 21 bp, Artificial Sequence
  Synthetic Construct
  misc_feature (20)..(21): deoxythymine (dT) base
cuuuacuucugguccaacatt

SEQ ID NO: 167 - DNA, 21 bp, Artificial Sequence
  Synthetic Construct
  misc_feature (20)..(21): deoxythymine (dT) base
augaggaugcauaggaccutt

SEQ ID NO: 168 - DNA, 21 bp, Artificial Sequence
  Synthetic Construct
gagaucagcggacaaagagtt

SEQ ID NO: 169 - DNA, 21 bp, Artificial Sequence
  Synthetic Construct
  misc_feature (20)..(21): deoxythymine (dT) base
gaaaggucaaaggcaaauutt

SEQ ID NO: 170 - DNA, 21 bp, Artificial Sequence
  Synthetic Construct
  misc_feature (20)..(21): deoxythymine (dT) base
agaaaggucaaaggcaaautt

SEQ ID NO: 171 - DNA, 21 bp, Artificial Sequence
  Synthetic Construct
  misc_feature (20)..(21): deoxythymine (dT) base
gcggacaaagagcaggaaatt

SEQ ID NO: 172 - DNA, 21 bp, Artificial Sequence
  Synthetic Construct
  misc_feature (20)..(21): deoxythymine (dT) base
ggaccugggcugagaagaatt

SEQ ID NO: 173 - DNA, 21 bp, Artificial Sequence
  Synthetic Construct
  misc_feature (20)..(21): deoxythymine (dT) base
aaagagcaggaaaagcagatt

SEQ ID NO: 174 - DNA, 21 bp, Artificial Sequence
  Synthetic Construct
  misc_feature (20)..(21): deoxythymine (dT) base
aagagcaggaaaagcagaatt

SEQ ID NO: 175 - DNA, 21 bp, Artificial Sequence
  Synthetic Construct
  misc_feature (20)..(21): deoxythymine (dT) base
aauggccccucggaaccuutt

SEQ ID NO: 176 - DNA, 21 bp, Artificial Sequence
  Synthetic Construct
  misc_feature (20)..(21): deoxythymine (dT) base
ccugaaacgguguccuuuutt

SEQ ID NO: 177 - DNA, 21 bp, Artificial Sequence
  Synthetic Construct
  misc_feature (20)..(21): deoxythymine (dT) base
cccugaaacgguguccuuutt

SEQ ID NO: 178 - DNA, 21 bp, Artificial Sequence
  Synthetic Construct
  misc_feature (20)..(21): deoxythymine (dT) base
uuuacuucugguccaacautt

SEQ ID NO: 179 - DNA, 21 bp, Artificial Sequence
  Synthetic Construct
  misc_feature (20)..(21): deoxythymine (dT) base
aggaccugggcugagaagatt

SEQ ID NO: 180 - DNA, 21 bp, Artificial Sequence
  Synthetic Construct
  misc_feature (20)..(21): deoxythymine (dT) base
ucaaaggcaaauugagacatt

SEQ ID NO: 181 - DNA, 21 bp, Artificial Sequence
  Synthetic Construct
  misc_feature (20)..(21): deoxythymine (dT) base
gaggaugcauaggaccuggtt

SEQ ID NO: 182 - DNA, 21 bp, Artificial Sequence
  Synthetic Construct
  misc_feature (20)..(21): deoxythymine (dT) base
caaagagcaggaaaagcagtt

SEQ ID NO: 183 - DNA, 21 bp, Artificial Sequence
  Synthetic Construct
  misc_feature (20)..(21): deoxythymine (dT) base
gaaaacaagucccuaagaatt

SEQ ID NO: 184 - DNA, 21 bp, Artificial Sequence
  Synthetic Construct
  misc_feature (20)..(21): deoxythymine (dT) base
auaacagcccugcauagautt

SEQ ID NO: 185 - DNA, 21 bp, Artificial Sequence
  Synthetic Construct
  misc_feature (20)..(21): deoxythymine (dT) base
aggcaaauugagacaagaatt

SEQ ID NO: 186 - DNA, 21 bp, Artificial Sequence
  Synthetic Construct
  misc_feature (20)..(21): deoxythymine (dT) base
aacauagcuuugagcuuuctt

SEQ ID NO: 187 - DNA, 21 bp, Artificial Sequence
  Synthetic Construct
  misc_feature (20)..(21): deoxythymine (dT) base
cauagcuuugagcuuucaatt

SEQ ID NO: 188 - DNA, 21 bp, Artificial Sequence
  Synthetic Construct
  misc_feature (20)..(21): deoxythymine (dT) base
agaagaccgggugcaaacctt

SEQ ID NO: 189 - DNA, 21 bp, Artificial Sequence
  Synthetic Construct
  misc_feature (20)..(21): deoxythymine (dT) base
agaacacuuuacuucuggutt

SEQ ID NO: 190 - DNA, 21 bp, Artificial Sequence
  Synthetic Construct
  misc_feature (20)..(21): deoxythymine (dT) base
aagaagaccgggugcaaactt

SEQ ID NO: 191 - DNA, 21 bp, Artificial Sequence
  Synthetic Construct
  misc_feature (20)..(21): deoxythymine (dT) base
gucaaaggcaaauugagactt

SEQ ID NO: 192 - DNA, 21 bp, Artificial Sequence
  Synthetic Construct
  misc_feature (20)..(21): deoxythymine (dT) base
uugagacaagaaggcaccutt

SEQ ID NO: 193 - DNA, 21 bp, Artificial Sequence
  Synthetic Construct
  misc_feature (20)..(21): deoxythymine (dT) base
aagggaggaguuuccucaatt

SEQ ID NO: 194 - DNA, 21 bp, Artificial Sequence
  Synthetic Construct
  misc_feature (20)..(21): deoxythymine (dT) base
uagaugaggaugcauaggatt

SEQ ID NO: 195 - DNA, 21 bp, Artificial Sequence
  Synthetic Construct
  misc_feature (20)..(21): deoxythymine (dT) base
acauagcuuugagcuuucatt

SEQ ID NO: 196 - DNA, 21 bp, Artificial Sequence
  Synthetic Construct
  misc_feature (20)..(21): deoxythymine (dT) base
gcaggaaaagcagaaaacatt

SEQ ID NO: 197 - DNA, 21 bp, Artificial Sequence
  deoxythymine (dT) base
  misc_feature (20)..(21): deoxythymine (dT) base
uugagcuuucaaagaagagtt

SEQ ID NO: 198 - DNA, 21 bp, Artificial Sequence
  Synthetic Construct
  misc_feature (20)..(21): deoxythymine (dT) base
auggccccucggaaccuuutt

SEQ ID NO: 199 - DNA, 21 bp, Artificial Sequence
  Synthetic Construct
  misc_feature (20)..(21): deoxythymine (dT) base
ggcaaauugagacaagaagtt

SEQ ID NO: 200 - DNA, 21 bp, Artificial Sequence
  Synthetic Construct
  misc_feature (20)..(21): deoxythymine (dT) base
agggaggaguuuccucaautt

SEQ ID NO: 201 - DNA, 21 bp, Artificial Sequence
  Synthetic Construct
  misc_feature (20)..(21): deoxythymine (dT) base
uuacuucugguccaacauatt

SEQ ID NO: 202 - DNA, 21 bp, Artificial Sequence
  Synthetic Construct
  misc_feature (20)..(21): deoxythymine (dT) base
ugagacaagaaggcaccugtt

SEQ ID NO: 203 - DNA, 21 bp, Artificial Sequence
  Synthetic Construct
  misc_feature (20)..(21): deoxythymine (dT) base
aaaggucaaaggcaaauugtt

SEQ ID NO: 204 - DNA, 21 bp, Artificial Sequence
  Synthetic Construct
  misc_feature (20)..(21): deoxythymine (dT) base
agaaacgagaacacuuuactt

SEQ ID NO: 205 - DNA, 21 bp, Artificial Sequence
  Synthetic Construct
  misc_feature (20)..(21): deoxythymine (dT) base
aggaugcauaggaccugggtt

SEQ ID NO: 206 - DNA, 21 bp, Artificial Sequence
  Synthetic Construct
  misc_feature (20)..(21): deoxythymine (dT) base
auagaugaggaugcauaggtt

SEQ ID NO: 207 - DNA, 21 bp, Artificial Sequence
  Synthetic Construct
  misc_feature (20)..(21): deoxythymine (dT) base
guguccuuuucggaagcuutt

SEQ ID NO: 208 - DNA, 21 bp, Artificial Sequence
  Synthetic Construct
  misc_feature (20)..(21): deoxythymine (dT) base
gcaagaagaccgggugcaatt

SEQ ID NO: 209 - DNA, 21 bp, Artificial Sequence
  Synthetic Construct
  misc_feature (20)..(21): deoxythymine (dT) base
ucccaggagaucagcggactt

SEQ ID NO: 210 - DNA, 21 bp, Artificial Sequence
  Synthetic Construct
  misc_feature (20)..(21): deoxythymine (dT) base
gccccucggaaccuuucaatt

SEQ ID NO: 211 - DNA, 21 bp, Artificial Sequence
  Synthetic Construct
  misc_feature (20)..(21): deoxythymine (dT) base
cccugcauagaugaggaugtt

SEQ ID NO: 212 - DNA, 21 bp, Artificial Sequence
  Synthetic Construct
  misc_feature (20)..(21): deoxythymine (dT) base
auugagacaagaaggcacctt

SEQ ID NO: 213 - DNA, 21 bp, Artificial Sequence
  Synthetic Construct
  misc_feature (20)..(21): deoxythymine (dT) base
cuaagaaaggucaaaggcatt

SEQ ID NO: 214 - DNA, 21 bp, Artificial Sequence
  Synthetic Construct
  misc_feature (20)..(21): deoxythymine (dT) base
gguccaacauagcuuugagtt

SEQ ID NO: 215 - DNA, 21 bp, Artificial Sequence
  Synthetic Construct
  misc_feature (20)..(21): deoxythymine (dT) base
gaacacuuuacuucugguctt

SEQ ID NO: 216 - DNA, 21 bp, Artificial Sequence
  Synthetic Construct
  misc_feature (20)..(21): deoxythymine (dT) base
uuucuucccaggagaucagtt

SEQ ID NO: 217 - DNA, 21 bp, Artificial Sequence
  Synthetic Construct
  misc_feature (20)..(21): deoxythymine (dT) base
agcggacaaagagcaggaatt

SEQ ID NO: 218 - DNA, 21 bp, Artificial Sequence
  Synthetic Construct
  misc_feature (20)..(21): deoxythymine (dT) base
ugaggaugcauaggaccugtt

SEQ ID NO: 219 - DNA, 21 bp, Artificial Sequence
  Synthetic Construct
  misc_feature (20)..(21): deoxythymine (dT) base
uucccaggagaucagcggatt

SEQ ID NO: 220 - DNA, 21 bp, Artificial Sequence
  Synthetic Construct
  misc_feature (20)..(21): deoxythymine (dT) base
aaaauaccacucgaugcuatt

SEQ ID NO: 221 - DNA, 21 bp, Artificial Sequence
  Synthetic Construct
  misc_feature (20)..(21): deoxythymine (dT) base
gcuuucuucccaggagauctt

SEQ ID NO: 222 - DNA, 21 bp, Artificial Sequence
  Synthetic Construct
  misc_feature (20)..(21): deoxythymine (dT) base
aaggucaaaggcaaauugatt

SEQ ID NO: 223 - DNA, 21 bp, Artificial Sequence
  Synthetic Construct
  misc_feature (20)..(21): deoxythymine (dT) base
uucugguccaacauagcuutt

SEQ ID NO: 224 - DNA, 21 bp, Artificial Sequence
  Synthetic Construct
  misc_feature (20)..(21): deoxythymine (dT) base
uaagaaaggucaaaggcaatt

SEQ ID NO: 225 - DNA, 21 bp, Artificial Sequence
  Synthetic Construct
  misc_feature (20)..(21): deoxythymine (dT) base
agucccuaagaaaggucaatt

SEQ ID NO: 226 - DNA, 21 bp, Artificial Sequence
  Synthetic Construct
  misc_feature (20)..(21): deoxythymine (dT) base
gaagaaaacgggacuaucatt

SEQ ID NO: 227 - DNA, 21 bp, Artificial Sequence
  Synthetic Construct
  misc_feature (20)..(21): deoxythymine (dT) base
uaacagcccugcauagaugtt

SEQ ID NO: 228 - DNA, 21 bp, Artificial Sequence
  Synthetic Construct
  misc_feature (20)..(21): deoxythymidine (dT) base
ugagcuuucaaagaagagatt

SEQ ID NO: 229 - DNA, 21 bp, Artificial Sequence
  Synthetic Construct
  misc_feature (20)..(21): deoxythymidine (dT) base
cuucugguccaacauagcutt

SEQ ID NO: 230 - DNA, 21 bp, Artificial Sequence
  deoxythymidine (dT) base
agagcaggaaaagcagaaatt

SEQ ID NO: 231 - DNA, 21 bp, Artificial Sequence
  Synthetic Construct
  misc_feature (20)..(21): deoxythymidine (dT) base
gagcaggaaaagcagaaaatt

SEQ ID NO: 232 - DNA, 21 bp, Artificial Sequence
  Synthetic Construct
  misc_feature (20)..(21): deoxythymidine (dT) base
ucuucccaggagaucagcgtt

SEQ ID NO: 233 - DNA, 21 bp, Artificial Sequence
  Synthetic Construct
  misc_feature (20)..(21): deoxythymidine (dT) base
acuucugguccaacauagctt

SEQ ID NO: 234 - DNA, 21 bp, Artificial Sequence
  Synthetic Construct
  misc_feature (20)..(21): deoxythymidine (dT) base
aauaccacucgaugcuacatt

SEQ ID NO: 235 - DNA, 21 bp, Artificial Sequence
  Synthetic Construct
  misc_feature (20)..(21): deoxythymidine (dT) base
ugaaacgguguccuuuucgtt

SEQ ID NO: 236 - DNA, 21 bp, Artificial Sequence
  Synthetic Construct
  misc_feature (20)..(21): deoxythymidine (dT) base
gguguccuuuucggaagcutt

SEQ ID NO: 237 - DNA, 21 bp, Artificial Sequence
  Synthetic Construct
  misc_feature (20)..(21): deoxythymidine (dT) base
cggaagcuuucuucccaggtt

SEQ ID NO: 238 - DNA, 21 bp, Artificial Sequence
  Synthetic Construct
  misc_feature (20)..(21): deoxythymidine (dT) base
cggacaaagagcaggaaaatt

SEQ ID NO: 239 - DNA, 21 bp, Artificial Sequence
  Synthetic Construct
  misc_feature (20)..(21): deoxythymidine (dT) base
gaucagcggacaaagagcatt

SEQ ID NO: 240 - DNA, 21 bp, Artificial Sequence
  Synthetic Construct
  misc_feature (20)..(21): deoxythymidine (dT) base
cacuuuacuucugguccaatt

SEQ ID NO: 241 - DNA, 21 bp, Artificial Sequence
  Synthetic Construct
  misc_feature (20)..(21): deoxythymidine (dT) base
aggagaucagcggacaaagtt

SEQ ID NO: 242 - DNA, 21 bp, Artificial Sequence
  Synthetic Construct
  misc_feature (20)..(21): deoxythymidine (dT) base
aaaggcaaauugagacaagtt

SEQ ID NO: 243 - DNA, 21 bp, Artificial Sequence
  Synthetic Construct
  misc_feature (20)..(21): deoxythymidine (dT) base
agggcaagaagaccgggugtt

SEQ ID NO: 244 - DNA, 21 bp, Artificial Sequence
  Synthetic Construct
  misc_feature (20)..(21): deoxythymidine (dT) base
ucccuaagaaaggucaaagtt

SEQ ID NO: 245 - DNA, 21 bp, Artificial Sequence
  Synthetic Construct
  misc_feature (20)..(21): deoxythymidine (dT) base
cccuaagaaaggucaaaggtt

SEQ ID NO: 246 - DNA, 21 bp, Artificial Sequence
  Synthetic Construct
  misc_feature (20)..(21): deoxythymidine (dT) base
uucggaagcuuucuucccatt

SEQ ID NO: 247 - DNA, 21 bp, Artificial Sequence
  Synthetic Construct
  misc_feature (20)..(21): deoxythymidine (dT) base
gauaacagcccugcauagatt

SEQ ID NO: 248 - DNA, 21 bp, Artificial Sequence
  Synthetic Construct
  misc_feature (20)..(21): deoxythymidine (dT) base
ucugguccaacauagcuuutt

SEQ ID NO: 249 - DNA, 21 bp, Artificial Sequence
  Synthetic Construct
  misc_feature (20)..(21): deoxythymidine (dT) base
ggucaaaggcaaauugagatt

SEQ ID NO: 250 - DNA, 21 bp, Artificial Sequence
  Synthetic Construct
  misc_feature (20)..(21): deoxythymidine (dT) base
cugcauagaugaggaugcatt

SEQ ID NO: 251 - DNA, 21 bp, Artificial Sequence
  Synthetic Construct
  misc_feature (20)..(21): deoxythymidine (dT) base
uggccccucggaaccuuuctt

SEQ ID NO: 252 - DNA, 21 bp, Artificial Sequence
  Synthetic Construct
  misc_feature (20)..(21): deoxythymidine (dT) base
caaauugagacaagaaggctt

SEQ ID NO: 253 - DNA, 21 bp, Artificial Sequence
  Synthetic Construct
  misc_feature (20)..(21): deoxythymidine (dT) base
gauauuaaaagggaggagutt

SEQ ID NO: 254 - DNA, 21 bp, Artificial Sequence
  Synthetic Construct
  misc_feature (20)..(21): deoxythymidine (dT) base
cuuuucggaagcuuucuuctt

SEQ ID NO: 255 - DNA, 21 bp, Artificial Sequence
  Synthetic Construct
  misc_feature (20)..(21): deoxythymidine (dT) base
aagucccuaagaaaggucatt

SEQ ID NO: 256 - DNA, 21 bp, Artificial Sequence
  Synthetic Construct
  misc_feature (20)..(21): deoxythymidine (dT) base
ccaacauagcuuugagcuutt

SEQ ID NO: 257 - DNA, 21 bp, Artificial Sequence
  Synthetic Construct
  misc_feature (20)..(21): deoxythymidine (dT) base
gggugcaaacccggaacugtt

SEQ ID NO: 258 - DNA, 21 bp, Artificial Sequence
  Synthetic Construct
  misc_feature (20)..(21): deoxythymidine (dT) base
ggcaagaagaccgggugcatt

SEQ ID NO: 259 - DNA, 21 bp, Artificial Sequence
  Synthetic Construct
  misc_feature (20)..(21): deoxythymidine (dT) base
augcguuaaacccaggaggtt

SEQ ID NO: 260 - DNA, 21 bp, Artificial Sequence
  Synthetic Construct
  misc_feature (20)..(21): deoxythymidine (dT) base
cgguguccuuuucggaagctt

SEQ ID NO: 261 - DNA, 21 bp, Artificial Sequence
  Synthetic Construct
  misc_feature (20)..(21): deoxythymidine (dT) base
aggucaaaggcaaauugagtt

SEQ ID NO: 262 - DNA, 21 bp, Artificial Sequence
  Synthetic Construct
  misc_feature (20)..(21): deoxythymidine (dT) base
gaugaggaugcauaggacctt

SEQ ID NO: 263 - DNA, 21 bp, Artificial Sequence
  Synthetic Construct
  misc_feature (20)..(21): deoxythymidine (dT) base
agcaggaaaagcagaaaactt

SEQ ID NO: 264 - DNA, 21 bp, Artificial Sequence
  Synthetic Construct
  misc_feature (20)..(21): deoxythymidine (dT) base
caggaaaagcagaaaacaatt

SEQ ID NO: 265 - DNA, 21 bp, Artificial Sequence
  Synthetic Construct
  misc_feature (20)..(21): deoxythymidine (dT) base
cgagaacacuuuacuucugtt

SEQ ID NO: 266 - DNA, 21 bp, Artificial Sequence
  Synthetic Construct
  misc_feature (20)..(21): deoxythymidine (dT) base
acaaagagcaggaaaagcatt

SEQ ID NO: 267 - DNA, 21 bp, Artificial Sequence
  Synthetic Construct
  misc_feature (20)..(21): deoxythymidine (dT) base
uucuucccaggagaucagctt

SEQ ID NO: 268 - DNA, 21 bp, Artificial Sequence
  Synthetic Construct
  misc_feature (20)..(21): deoxythymidine (dT) base
cucgccccugaaacggugutt

SEQ ID NO: 269 - DNA, 21 bp, Artificial Sequence
  Synthetic Construct
  misc_feature (20)..(21): deoxythymidine (dT) base
cugaaacgguguccuuuuctt

SEQ ID NO: 270 - DNA, 21 bp, Artificial Sequence
  Synthetic Construct
  misc_feature (20)..(21): deoxythymidine (dT) base
gaccugggcugagaagaaatt

SEQ ID NO: 271 - DNA, 21 bp, Artificial Sequence
  Synthetic Construct
  misc_feature (20)..(21): deoxythymidine (dT) base
aacagcccugcauagaugatt

SEQ ID NO: 272 - DNA, 21 bp, Artificial Sequence
  Synthetic Construct
  misc_feature (20)..(21): deoxythymidine (dT) base
aaagggaggaguuuccucatt

SEQ ID NO: 273 - DNA, 21 bp, Artificial Sequence
  Synthetic Construct
  misc_feature (20)..(21): deoxythymidine (dT) base
aguuuccucaauggccccutt

SEQ ID NO: 274 - DNA, 21 bp, Artificial Sequence
  Synthetic Construct
  misc_feature (20)..(21): deoxythymidine (dT) base
aagaaaggucaaaggcaaatt

SEQ ID NO: 275 - DNA, 21 bp, Artificial Sequence
  Synthetic Construct
  misc_feature (20)..(21): deoxythymidine (dT) base
ccaggagaucagcggacaatt

SEQ ID NO: 276 - DNA, 21 bp, Artificial Sequence
  Synthetic Construct
  misc_feature (20)..(21): deoxythymidine (dT) base
auaggaccugggcugagaatt

SEQ ID NO: 277 - DNA, 21 bp, Artificial Sequence
  Synthetic Construct
  misc_feature (20)..(21): deoxythymidine (dT) base
aauugagacaagaaggcactt

SEQ ID NO: 278 - DNA, 21 bp, Artificial Sequence
  Synthetic Construct
  misc_feature (20)..(21): deoxythymidine (dT) base
aucacaaugcguuaaaccctt

SEQ ID NO: 279 - DNA, 21 bp, Artificial Sequence
  Synthetic Construct
  misc_feature (20)..(21): deoxythymidine (dT) base
acacuuuacuucugguccatt

SEQ ID NO: 280 - DNA, 21 bp, Artificial Sequence
  Synthetic Construct
  misc_feature (20)..(21): deoxythymidine (dT) base
caaaggcaaauugagacaatt

SEQ ID NO: 281 - DNA, 21 bp, Artificial Sequence
  Synthetic Construct
  misc_feature (20)..(21): deoxythymidine (dT) base
uuucggaagcuuucuuccctt

SEQ ID NO: 282 - DNA, 21 bp, Artificial Sequence
  Synthetic Construct
  misc_feature (20)..(21): deoxythymidine (dT) base
ggaugcauaggaccugggctt

SEQ ID NO: 283 - DNA, 21 bp, Artificial Sequence
  Synthetic Construct
  misc_feature (20)..(21): deoxythymidine (dT) base
caagaagaccgggugcaaatt

SEQ ID NO: 284 - DNA, 21 bp, Artificial Sequence
  Synthetic Construct
  misc_feature (20)..(21): deoxythymidine (dT) base
ucggaagcuuucuucccagtt

SEQ ID NO: 285 - DNA, 21 bp, Artificial Sequence
  Synthetic Construct
  misc_feature (20)..(21): deoxythymidine (dT) base
ggugcaaacccggaacuggtt

SEQ ID NO: 286 - DNA, 21 bp, Artificial Sequence
  Synthetic Construct
  misc_feature (20)..(21): deoxythymidine (dT) base
ccugggcugagaagaaaactt

SEQ ID NO: 287 - DNA, 21 bp, Artificial Sequence
  Synthetic Construct
  misc_feature (20)..(21): deoxythymidine (dT) base
aaggcaaauugagacaagatt

SEQ ID NO: 288 - DNA, 21 bp, Artificial Sequence
  Synthetic Construct
  misc_feature (20)..(21): deoxythymidine (dT) base
agaugaggaugcauaggactt

SEQ ID NO: 289 - DNA, 21 bp, Artificial Sequence
  Synthetic Construct
  misc_feature (20)..(21): deoxythymidine (dT) base
aacgguguccuuuucggaatt

SEQ ID NO: 290 - DNA, 21 bp, Artificial Sequence
  Synthetic Construct
  misc_feature (20)..(21): deoxythymidine (dT) base
accugggcugagaagaaaatt

SEQ ID NO: 291 - DNA, 21 bp, Artificial Sequence
  Synthetic Construct
  misc_feature (20)..(21): deoxythymidine (dT) base
aacgggacuaucacaaugctt

SEQ ID NO: 292 - DNA, 21 bp, Artificial Sequence
  Synthetic Construct
  misc_feature (20)..(21): deoxythymidine (dT) base
cccucggaaccuuucaagutt

SEQ ID NO: 293 - DNA, 21 bp, Artificial Sequence
  Synthetic Construct
  misc_feature (20)..(21): deoxythymidine (dT) base
ucaauggccccucggaacctt

SEQ ID NO: 294 - DNA, 21 bp, Artificial Sequence
  Synthetic Construct
  misc_feature (20)..(21): deoxythymidine (dT) base
gcaaauugagacaagaaggtt

SEQ ID NO: 295 - DNA, 21 bp, Artificial Sequence
  Synthetic Construct
  misc_feature (20)..(21): deoxythymidine (dT) base
ucacaaugcguuaaacccatt

SEQ ID NO: 296 - DNA, 21 bp, Artificial Sequence
  Synthetic Construct
  misc_feature (20)..(21): deoxythymidine (dT) base
gucccuaagaaaggucaaatt

SEQ ID NO: 297 - DNA, 21 bp, Artificial Sequence
  Synthetic Construct
  misc_feature (20)..(21): deoxythymidiine (dT) base
aagaaaacgggacuaucactt

SEQ ID NO: 298 - DNA, 21 bp, Artificial Sequence
  Synthetic Construct
  misc_feature (20)..(21): deoxythymidine (dT) base
agacaagaaggcaccuguatt

SEQ ID NO: 299 - DNA, 21 bp, Artificial Sequence
  Synthetic Construct
  misc_feature (20)..(21): deoxythymidine (dT) base
agaaaacgggacuaucacatt

SEQ ID NO: 300 - DNA, 21 bp, Artificial Sequence
  Synthetic Construct
  misc_feature (20)..(21): deoxythymidine (dT) base
uaugcagauaacagcccugtt

SEQ ID NO: 301 - DNA, 21 bp, Artificial Sequence
  Synthetic Construct
  misc_feature (20)..(21): deoxythymidine (dT) base
ucacucgccccugaaacggtt

SEQ ID NO: 302 - DNA, 21 bp, Artificial Sequence
  Synthetic Construct
  misc_feature (20)..(21): deoxythymidine (dT) base
gaggaguuuccucaauggctt

SEQ ID NO: 303 - DNA, 21 bp, Artificial Sequence
  Synthetic Construct
  misc_feature (20)..(21): deoxythymidine (dT) base
ggaggaguuuccucaauggtt

SEQ ID NO: 304 - DNA, 21 bp, Artificial Sequence
  Synthetic Construct
  misc_feature (20)..(21): deoxythymidine (dT) base
gcauaggaccugggcugagtt

SEQ ID NO: 305 - DNA, 21 bp, Artificial Sequence
  Synthetic Construct
  misc_feature (20)..(21): deoxythymidine (dT) base
cuuucuucccaggagaucatt

SEQ ID NO: 306 - DNA, 21 bp, Artificial Sequence
  Synthetic Construct
  misc_feature (20)..(21): deoxythymidine (dT) base
uaccacucgaugcuacauutt

SEQ ID NO: 307 - DNA, 21 bp, Artificial Sequence
  Synthetic Construct
  misc_feature (20)..(21): deoxythymidine (dT) base
cuuugagcuuucaaagaagtt

SEQ ID NO: 308 - DNA, 21 bp, Artificial Sequence
  Synthetic Construct
  misc_feature (20)..(21): deoxythymidine (dT) base
ccccugaaacgguguccuutt

SEQ ID NO: 309 - DNA, 21 bp, Artificial Sequence
  Synthetic Construct
  misc_feature (20)..(21): deoxythymidine (dT) base
gaaacgguguccuuuucggtt

SEQ ID NO: 310 - DNA, 21 bp, Artificial Sequence
  Synthetic Construct
  misc_feature (20)..(21): deoxythymidine (dT) base
ccucggaaccuuucaaguutt

SEQ ID NO: 311 - DNA, 21 bp, Artificial Sequence
  Synthetic Construct
  misc_feature (20)..(21): deoxythymidine (dT) base
gagaacacuuuacuucuggtt

SEQ ID NO: 312 - DNA, 21 bp, Artificial Sequence
  Synthetic Construct
  misc_feature (20)..(21): deoxythymidine (dT) base
gcugagaagaaaacgggactt

SEQ ID NO: 313 - DNA, 21 bp, Artificial Sequence
  Synthetic Construct
  misc_feature (20)..(21): deoxythymidine (dT) base
cagcccugcauagaugaggtt

SEQ ID NO: 314 - DNA, 21 bp, Artificial Sequence
  Synthetic Construct
  misc_feature (20)..(21): deoxythymidine (dT) base
guccuuuucggaagcuuuctt

SEQ ID NO: 315 - DNA, 21 bp, Artificial Sequence
  Synthetic Construct
  misc_feature (20)..(21): deoxythymidine (dT) base
cagcggacaaagagcaggatt

SEQ ID NO: 316 - DNA, 21 bp, Artificial Sequence
  Synthetic Construct
  misc_feature (20)..(21): deoxythymidine (dT) base
aacaagucccuaagaaaggtt

SEQ ID NO: 317 - DNA, 21 bp, Artificial Sequence
  Synthetic Construct
  misc_feature (20)..(21): deoxythymidine (dT) base
cagaaaacaagucccuaagtt

SEQ ID NO: 318 - DNA, 21 bp, Artificial Sequence
  Synthetic Construct
  misc_feature (20)..(21): deoxythymidine (dT) base
guuaaacccaggagguuuutt

SEQ ID NO: 319 - DNA, 21 bp, Artificial Sequence
  Synthetic Construct
  misc_feature (20)..(21): deoxythymidine (dT) base
gccccugaaacgguguccutt

SEQ ID NO: 320 - DNA, 21 bp, Artificial Sequence
  Synthetic Construct
  misc_feature (20)..(21): deoxythymidine (dT) base
uccuuuucggaagcuuucutt

SEQ ID NO: 321 - DNA, 21 bp, Artificial Sequence
  Synthetic Construct
  misc_feature (20)..(21): deoxythymidine (dT) base
uaaaagggaggaguuuccutt

SEQ ID NO: 322 - DNA, 21 bp, Artificial Sequence
  Synthetic Construct
  misc_feature (20)..(21): deoxythymidine (dT) base
uuuccucaauggccccucgtt

SEQ ID NO: 323 - DNA, 21 bp, Artificial Sequence
  Synthetic Construct
  misc_feature (20)..(21): deoxythymidine (dT) base
acaaugcguuaaacccaggtt

SEQ ID NO: 324 - DNA, 21 bp, Artificial Sequence
  Synthetic Construct
  misc_feature (20)..(21): deoxythymidine (dT) base
uagcuuugagcuuucaaagtt

SEQ ID NO: 325 - DNA, 21 bp, Artificial Sequence
  Synthetic Construct
  misc_feature (20)..(21): deoxythymidine (dT) base
acgagaacacuuuacuucutt

SEQ ID NO: 326 - DNA, 21 bp, Artificial Sequence
  Synthetic Construct
  misc_feature (20)..(21): deoxythymidine (dT) base
ugcauagaugaggaugcautt

SEQ ID NO: 327 - DNA, 21 bp, Artificial Sequence
  Synthetic Construct
  misc_feature (20)..(21): deoxythymidine (dT) base
ccccucggaaccuuucaagtt

SEQ ID NO: 328 - DNA, 21 bp, Artificial Sequence
  Synthetic Construct
  misc_feature (20)..(21): deoxythymidine (dT) base
gacaaagagcaggaaaagctt

SEQ ID NO: 329 - DNA, 19 bp, Artificial Sequence
  Synthetic Construct
gctctttgtccgctgatct

SEQ ID NO: 330 - DNA, 19 bp, Artificial Sequence
  Synthetic Construct
tgttggaccagaagtaaag

SEQ ID NO: 331 - DNA, 19 bp, Artificial Sequence
  Synthetic Construct
aggtcctatgcatcctcat

SEQ ID NO: 332 - DNA, 19 bp, Artificial Sequence
  Synthetic Construct
ctctttgtccgctgatctc

SEQ ID NO: 333 - DNA, 19 bp, Artificial Sequence
  Synthetic Construct
aatttgcctttgacctttc

SEQ ID NO: 334 - DNA, 19 bp, Artificial Sequence
  Synthetic Construct
atttgcctttgacctttct

SEQ ID NO: 335 - DNA, 19 bp, Artificial Sequence
  Synthetic Construct
tttcctgctctttgtccgc

SEQ ID NO: 336 - DNA, 19 bp, Artificial Sequence
  Synthetic Construct
ttcttctcagcccaggtcc

SEQ ID NO: 337 - DNA, 19 bp, Artificial Sequence
  Synthetic Construct
tctgcttttcctgctcttt

SEQ ID NO: 338 - DNA, 19 bp, Artificial Sequence
  Synthetic Construct
ttctgcttttcctgctctt

SEQ ID NO: 339 - DNA, 19 bp, Artificial Sequence
  Synthetic Construct
aaggttccgaggggccatt

SEQ ID NO: 340 - DNA, 19 bp, Artificial Sequence
  Synthetic Construct
aaaaggacaccgtttcagg

SEQ ID NO: 341 - DNA, 19 bp, Artificial Sequence
  Synthetic Construct
aaaggacaccgtttcaggg

SEQ ID NO: 342 - DNA, 19 bp, Artificial Sequence
  Synthetic Construct
atgttggaccagaagtaaa

SEQ ID NO: 343 - DNA, 19 bp, Artificial Sequence
  Synthetic Construct
tcttctcagcccaggtcct

SEQ ID NO: 344 - DNA, 19 bp, Artificial Sequence
  Synthetic Construct
  misc_feature (20)..(21): deoxythymidine (dT) base
tgtctcaatttgcctttga

SEQ ID NO: 345 - DNA, 19 bp, Artificial Sequence
  Synthetic Construct
  misc_feature (20)..(21): deoxythymidine (dT) base
ccaggtcctatgcatcctc

SEQ ID NO: 346 - DNA, 19 bp, Artificial Sequence
  Synthetic Construct
ctgcttttcctgctctttg

SEQ ID NO: 347 - DNA, 19 bp, Artificial Sequence
  Synthetic Construct
ttcttagggacttgttttc

SEQ ID NO: 348 - DNA, 19 bp, Artificial Sequence
  Synthetic Construct
atctatgcagggctgttat

SEQ ID NO: 349 - DNA, 19 bp, Artificial Sequence
  Synthetic Construct
ttcttgtctcaatttgcct

SEQ ID NO: 350 - DNA, 19 bp, Artificial Sequence
  Synthetic Construct
gaaagctcaaagctatgtt

SEQ ID NO: 351 - DNA, 19 bp, Artificial Sequence
  Synthetic Construct
ttgaaagctcaaagctatg

SEQ ID NO: 352 - DNA, 19 bp, Artificial Sequence
  Synthetic Construct
ggtttgcacccggtcttct

SEQ ID NO: 353 - DNA, 19 bp, Artificial Sequence
  Synthetic Construct
accagaagtaaagtgttct

SEQ ID NO: 354 - DNA, 19 bp, Artificial Sequence
  Synthetic Construct
gtttgcacccggtcttctt

SEQ ID NO: 355 - DNA, 19 bp, Artificial Sequence
  Synthetic Construct
gtctcaatttgcctttgac

SEQ ID NO: 356 - DNA, 19 bp, Artificial Sequence
  Synthetic Construct
aggtgccttcttgtctcaa

SEQ ID NO: 357 - DNA, 19 bp, Artificial Sequence
  Synthetic Construct
ttgaggaaactcctccctt

SEQ ID NO: 358 - DNA, 19 bp, Artificial Sequence
  Synthetic Construct
tcctatgcatcctcatcta

SEQ ID NO: 359 - DNA, 19 bp, Artificial Sequence
  Synthetic Construct
tgaaagctcaaagctatgt

SEQ ID NO: 360 - DNA, 19 bp, Artificial Sequence
  Synthetic Construct
tgttttctgcttttcctgc

SEQ ID NO: 361 - DNA, 19 bp, Artificial Sequence
  Synthetic Construct
ctcttctttgaaagctcaa

SEQ ID NO: 362 - DNA, 19 bp, Artificial Sequence
  Synthetic Construct
aaaggttccgaggggccat

SEQ ID NO: 363 - DNA, 19 bp, Artificial Sequence
  Synthetic Construct
cttcttgtctcaatttgcc

SEQ ID NO: 364 - DNA, 19 bp, Artificial Sequence
  Synthetic Construct
attgaggaaactcctccct

SEQ ID NO: 365 - DNA, 19 bp, Artificial Sequence
  Synthetic Construct
tatgttggaccagaagtaa

SEQ ID NO: 366 - DNA, 19 bp, Artificial Sequence
  Synthetic Construct
caggtgccttcttgtctca

SEQ ID NO: 367 - DNA, 19 bp, Artificial Sequence
  Synthetic Construct
caatttgcctttgaccttt

SEQ ID NO: 368 - DNA, 19 bp, Artificial Sequence
  Synthetic Construct
gtaaagtgttctcgtttct

SEQ ID NO: 369 - DNA, 19 bp, Artificial Sequence
  Synthetic Construct
cccaggtcctatgcatcct

SEQ ID NO: 370 - DNA, 19 bp, Artificial Sequence
  Synthetic Construct
cctatgcatcctcatctat

SEQ ID NO: 371 - DNA, 19 bp, Artificial Sequence
  Synthetic Construct
aagcttccgaaaaggacac

SEQ ID NO: 372 - DNA, 19 bp, Artificial Sequence
  Synthetic Construct
ttgcacccggtcttcttgc

SEQ ID NO: 373 - DNA, 19 bp, Artificial Sequence
  Synthetic Construct
gtccgctgatctcctggga

SEQ ID NO: 374 - DNA, 19 bp, Artificial Sequence
  Synthetic Construct
ttgaaaggttccgaggggc

SEQ ID NO: 375 - DNA, 19 bp, Artificial Sequence
  Synthetic Construct
catcctcatctatgcaggg

SEQ ID NO: 376 - DNA, 19 bp, Artificial Sequence
  Synthetic Construct
ggtgccttcttgtctcaat

SEQ ID NO: 377 - DNA, 19 bp, Artificial Sequence
  Synthetic Construct
tgcctttgacctttcttag

SEQ ID NO: 378 - DNA, 19 bp, Artificial Sequence
  Synthetic Construct
ctcaaagctatgttggacc

SEQ ID NO: 379 - DNA, 19 bp, Artificial Sequence
  Synthetic Construct
gaccagaagtaaagtgttc

SEQ ID NO: 380 - DNA, 19 bp, Artificial Sequence
  Synthetic Construct
ctgatctcctgggaagaaa

SEQ ID NO: 381 - DNA, 19 bp, Artificial Sequence
  Synthetic Construct
ttcctgctctttgtccgct

SEQ ID NO: 382 - DNA, 19 bp, Artificial Sequence
  Synthetic Construct
caggtcctatgcatcctca

SEQ ID NO: 383 - DNA, 19 bp, Artificial Sequence
  Synthetic Construct
tccgctgatctcctgggaa

SEQ ID NO: 384 - DNA, 19 bp, Artificial Sequence
  Synthetic Construct
tagcatcgagtggtatttt

SEQ ID NO: 385 - DNA, 19 bp, Artificial Sequence
  Synthetic Construct
gatctcctgggaagaaagc

SEQ ID NO: 386 - DNA, 19 bp, Artificial Sequence
  Synthetic Construct
tcaatttgcctttgacctt

SEQ ID NO: 387 - DNA, 19 bp, Artificial Sequence
  Synthetic Construct
aagctatgttggaccagaa

SEQ ID NO: 388 - DNA, 19 bp, Artificial Sequence
  Synthetic Construct
ttgcctttgacctttctta

SEQ ID NO: 389 - DNA, 19 bp, Artificial Sequence
  Synthetic Construct
ttgacctttcttagggact

SEQ ID NO: 390 - DNA, 19 bp, Artificial Sequence
  Synthetic Construct
tgatagtcccgttttcttc

SEQ ID NO: 391 - DNA, 19 bp, Artificial Sequence
  Synthetic Construct
catctatgcagggctgtta

SEQ ID NO: 392 - DNA, 19 bp, Artificial Sequence
  Synthetic Construct
tctcttctttgaaagctca

SEQ ID NO: 393 - DNA, 19 bp, Artificial Sequence
  Synthetic Construct
agctatgttggaccagaag

SEQ ID NO: 394 - DNA, 19 bp, Artificial Sequence
  Synthetic Construct
tttctgcttttcctgctct

SEQ ID NO: 395 - DNA, 19 bp, Artificial Sequence
  Synthetic Construct
ttttctgcttttcctgctc

SEQ ID NO: 396 - DNA, 19 bp, Artificial Sequence
  Synthetic Construct
cgctgatctcctgggaaga

SEQ ID NO: 397 - DNA, 19 bp, Artificial Sequence
  Synthetic Construct
gctatgttggaccagaagt

SEQ ID NO: 398 - DNA, 19 bp, Artificial Sequence
  Synthetic Construct
tgtagcatcgagtggtatt

SEQ ID NO: 399 - DNA, 19 bp, Artificial Sequence
  Synthetic Construct
cgaaaaggacaccgtttca

SEQ ID NO: 400 - DNA, 19 bp, Artificial Sequence
  Synthetic Construct
agcttccgaaaaggacacc

SEQ ID NO: 401 - DNA, 19 bp, Artificial Sequence
  Synthetic Construct
cctgggaagaaagcttccg

SEQ ID NO: 402 - DNA, 19 bp, Artificial Sequence
  Synthetic Construct
ttttcctgctctttgtccg

SEQ ID NO: 403 - DNA, 19 bp, Artificial Sequence
  Synthetic Construct
tgctctttgtccgctgatc

SEQ ID NO: 404 - DNA, 19 bp, Artificial Sequence
  Synthetic Construct
ttggaccagaagtaaagtg

SEQ ID NO: 405 - DNA, 19 bp, Artificial Sequence
  Synthetic Construct
ctttgtccgctgatctcct

SEQ ID NO: 406 - DNA, 19 bp, Artificial Sequence
  Synthetic Construct
cttgtctcaatttgccttt

SEQ ID NO: 407 - DNA, 19 bp, Artificial Sequence
  Synthetic Construct
cacccggtcttcttgccct

SEQ ID NO: 408 - DNA, 19 bp, Artificial Sequence
  Synthetic Construct
ctttgacctttcttaggga

SEQ ID NO: 409 - DNA, 19 bp, Artificial Sequence
  Synthetic Construct
cctttgacctttcttaggg

SEQ ID NO: 410 - DNA, 19 bp, Artificial Sequence
  Synthetic Construct
tgggaagaaagcttccgaa

SEQ ID NO: 411 - DNA, 19 bp, Artificial Sequence
  Synthetic Construct
tctatgcagggctgttatc

SEQ ID NO: 412 - DNA, 19 bp, Artificial Sequence
  Synthetic Construct
aaagctatgttggaccaga

SEQ ID NO: 413 - DNA, 19 bp, Artificial Sequence
  Synthetic Construct
tctcaatttgcctttgacc

SEQ ID NO: 414 - DNA, 19 bp, Artificial Sequence
  Synthetic Construct
tgcatcctcatctatgcag

SEQ ID NO: 415 - DNA, 19 bp, Artificial Sequence
  Synthetic Construct
gaaaggttccgaggggcca

SEQ ID NO: 416 - DNA, 19 bp, Artificial Sequence
  Synthetic Construct
gccttcttgtctcaatttg

SEQ ID NO: 417 - DNA, 19 bp, Artificial Sequence
  Synthetic Construct
actcctcccttttaatatc

SEQ ID NO: 418 - DNA, 19 bp, Artificial Sequence
  Synthetic Construct
gaagaaagcttccgaaaag

SEQ ID NO: 419 - DNA, 19 bp, Artificial Sequence
  Synthetic Construct
tgacctttcttagggactt

SEQ ID NO: 420 - DNA, 19 bp, Artificial Sequence
  Synthetic Construct
aagctcaaagctatgttgg

SEQ ID NO: 421 - DNA, 19 bp, Artificial Sequence
  Synthetic Construct
cagttccgggtttgcaccc

SEQ ID NO: 422 - DNA, 19 bp, Artificial Sequence
  Synthetic Construct
tgcacccggtcttcttgcc

SEQ ID NO: 423 - DNA, 19 bp, Artificial Sequence
  Synthetic Construct
cctcctgggtttaacgcat

SEQ ID NO: 424 - DNA, 19 bp, Artificial Sequence
  Synthetic Construct
gcttccgaaaaggacaccg

SEQ ID NO: 425 - DNA, 19 bp, Artificial Sequence
  Synthetic Construct
ctcaatttgcctttgacct

SEQ ID NO: 426 - DNA, 19 bp, Artificial Sequence
  Synthetic Construct
ggtcctatgcatcctcatc

SEQ ID NO: 427 - DNA, 19 bp, Artificial Sequence
  Synthetic Construct
gttttctgcttttcctgct

SEQ ID NO: 428 - DNA, 19 bp, Artificial Sequence
  Synthetic Construct
ttgttttctgcttttcctg

SEQ ID NO: 429 - DNA, 19 bp, Artificial Sequence
  Synthetic Construct
cagaagtaaagtgttctcg

SEQ ID NO: 430 - DNA, 19 bp, Artificial Sequence
  Synthetic Construct
tgcttttcctgctctttgt

SEQ ID NO: 431 - DNA, 19 bp, Artificial Sequence
  Synthetic Construct
gctgatctcctgggaagaa

SEQ ID NO: 432 - DNA, 19 bp, Artificial Sequence
  Synthetic Construct
acaccgtttcaggggcgag

SEQ ID NO: 433 - DNA, 19 bp, Artificial Sequence
  Synthetic Construct
gaaaaggacaccgtttcag

SEQ ID NO: 434 - DNA, 19 bp, Artificial Sequence
  Synthetic Construct
tttcttctcagcccaggtc

SEQ ID NO: 435 - DNA, 19 bp, Artificial Sequence
  Synthetic Construct
tcatctatgcagggctgtt

SEQ ID NO: 436 - DNA, 19 bp, Artificial Sequence
  Synthetic Construct
tgaggaaactcctcccttt

SEQ ID NO: 437 - DNA, 19 bp, Artificial Sequence
  Synthetic Construct
aggggccattgaggaaact

SEQ ID NO: 438 - DNA, 19 bp, Artificial Sequence
  Synthetic Construct
tttgcctttgacctttctt

SEQ ID NO: 439 - DNA, 19 bp, Artificial Sequence
  Synthetic Construct
ttgtccgctgatctcctgg

SEQ ID NO: 440 - DNA, 19 bp, Artificial Sequence
  Synthetic Construct
ttctcagcccaggtcctat

SEQ ID NO: 441 - DNA, 19 bp, Artificial Sequence
  Synthetic Construct
gtgccttcttgtctcaatt

SEQ ID NO: 442 - DNA, 19 bp, Artificial Sequence
  Synthetic Construct
gggtttaacgcattgtgat

SEQ ID NO: 443 - DNA, 19 bp, Artificial Sequence
  Synthetic Construct
tggaccagaagtaaagtgt

SEQ ID NO: 444 - DNA, 19 bp, Artificial Sequence
  Synthetic Construct
ttgtctcaatttgcctttg

SEQ ID NO: 445 - DNA, 19 bp, Artificial Sequence
  Synthetic Construct
gggaagaaagcttccgaaa

SEQ ID NO: 446 - DNA, 19 bp, Artificial Sequence
  Synthetic Construct
gcccaggtcctatgcatcc

SEQ ID NO: 447 - DNA, 19 bp, Artificial Sequence
  Synthetic Construct
tttgcacccggtcttcttg

SEQ ID NO: 448 - DNA, 19 bp, Artificial Sequence
  Synthetic Construct
ctgggaagaaagcttccga

SEQ ID NO: 449 - DNA, 19 bp, Artificial Sequence
  Synthetic Construct
ccagttccgggtttgcacc

SEQ ID NO: 450 - DNA, 19 bp, Artificial Sequence
  Synthetic Construct
gttttcttctcagcccagg

SEQ ID NO: 451 - DNA, 19 bp, Artificial Sequence
  Synthetic Construct
tcttgtctcaatttgcctt

SEQ ID NO: 452 - DNA, 19 bp, Artificial Sequence
  Synthetic Construct
gtcctatgcatcctcatct

SEQ ID NO: 453 - DNA, 19 bp, Artificial Sequence
  Synthetic Construct
ttccgaaaaggacaccgtt

SEQ ID NO: 454 - DNA, 19 bp, Artificial Sequence
  Synthetic Construct
ttttcttctcagcccaggt

SEQ ID NO: 455 - DNA, 19 bp, Artificial Sequence
  Synthetic Construct
gcattgtgatagtcccgtt

SEQ ID NO: 456 - DNA, 19 bp, Artificial Sequence
  Synthetic Construct
acttgaaaggttccgaggg

SEQ ID NO: 457 - DNA, 19 bp, Artificial Sequence
  Synthetic Construct
ggttccgaggggccattga

SEQ ID NO: 458 - DNA, 19 bp, Artificial Sequence
  Synthetic Construct
ccttcttgtctcaatttgc

SEQ ID NO: 459 - DNA, 19 bp, Artificial Sequence
  Synthetic Construct
tgggtttaacgcattgtga

SEQ ID NO: 460 - DNA, 19 bp, Artificial Sequence
  Synthetic Construct
tttgacctttcttagggac

SEQ ID NO: 461 - DNA, 19 bp, Artificial Sequence
  Synthetic Construct
gtgatagtcccgttttctt

SEQ ID NO: 462 - DNA, 19 bp, Artificial Sequence
  Synthetic Construct
tacaggtgccttcttgtct

SEQ ID NO: 463 - DNA, 19 bp, Artificial Sequence
  Synthetic Construct
tgtgatagtcccgttttct

SEQ ID NO: 464 - DNA, 19 bp, Artificial Sequence
  Synthetic Construct
cagggctgttatctgcata

SEQ ID NO: 465 - DNA, 19 bp, Artificial Sequence
  Synthetic Construct
ccgtttcaggggcgagtga

SEQ ID NO: 466 - DNA, 19 bp, Artificial Sequence
  Synthetic Construct
gccattgaggaaactcctc

SEQ ID NO: 467 - DNA, 19 bp, Artificial Sequence
  Synthetic Construct
ccattgaggaaactcctcc

SEQ ID NO: 468 - DNA, 19 bp, Artificial Sequence
  Synthetic Construct
ctcagcccaggtcctatgc

SEQ ID NO: 469 - DNA, 19 bp, Artificial Sequence
  Synthetic Construct
tgatctcctgggaagaaag

SEQ ID NO: 470 - DNA, 19 bp, Artificial Sequence
  Synthetic Construct
aatgtagcatcgagtggta

SEQ ID NO: 471 - DNA, 19 bp, Artificial Sequence
  Synthetic Construct
cttctttgaaagctcaaag

SEQ ID NO: 472 - DNA, 19 bp, Artificial Sequence
  Synthetic Construct
aaggacaccgtttcagggg

SEQ ID NO: 473 - DNA, 19 bp, Artificial Sequence
  Synthetic Construct
ccgaaaaggacaccgtttc

SEQ ID NO: 474 - DNA, 19 bp, Artificial Sequence
  Synthetic Construct
aacttgaaaggttccgagg

SEQ ID NO: 475 - DNA, 19 bp, Artificial Sequence
  Synthetic Construct
ccagaagtaaagtgttctc

SEQ ID NO: 476 - DNA, 19 bp, Artificial Sequence
  Synthetic Construct
gtcccgttttcttctcagc

SEQ ID NO: 477 - DNA, 19 bp, Artificial Sequence
  Synthetic Construct
cctcatctatgcagggctg

SEQ ID NO: 478 - DNA, 19 bp, Artificial Sequence
  Synthetic Construct
gaaagcttccgaaaaggac

SEQ ID NO: 479 - DNA, 19 bp, Artificial Sequence
  Synthetic Construct
tcctgctctttgtccgctg

SEQ ID NO: 480 - DNA, 19 bp, Artificial Sequence
  Synthetic Construct
cctttcttagggacttgtt

SEQ ID NO: 481 - DNA, 19 bp, Artificial Sequence
  Synthetic Construct
cttagggacttgttttctg

SEQ ID NO: 482 - DNA, 19 bp, Artificial Sequence
  Synthetic Construct
aaaacctcctgggtttaac

SEQ ID NO: 483 - DNA, 19 bp, Artificial Sequence
  Synthetic Construct
aggacaccgtttcaggggc

SEQ ID NO: 484 - DNA, 19 bp, Artificial Sequence
  Synthetic Construct
agaaagcttccgaaaagga

SEQ ID NO: 485 - DNA, 19 bp, Artificial Sequence
  Synthetic Construct
aggaaactcctccctttta

SEQ ID NO: 486 - DNA, 19 bp, Artificial Sequence
  Synthetic Construct
cgaggggccattgaggaaa

SEQ ID NO: 487 - DNA, 19 bp, Artificial Sequence
  Synthetic Construct
cctgggtttaacgcattgt

SEQ ID NO: 488 - DNA, 19 bp, Artificial Sequence
  Synthetic Construct
ctttgaaagctcaaagcta

SEQ ID NO: 489 - DNA, 19 bp, Artificial Sequence
  Synthetic Construct
agaagtaaagtgttctcgt

SEQ ID NO: 490 - DNA, 19 bp, Artificial Sequence
  Synthetic Construct
atgcatcctcatctatgca

SEQ ID NO: 491 - DNA, 19 bp, Artificial Sequence
  Synthetic Construct
cttgaaaggttccgagggg

SEQ ID NO: 492 - DNA, 19 bp, Artificial Sequence
  Synthetic Construct
gcttttcctgctctttgtc

SEQ ID NO: 493 - DNA, 1000 bp, Artificial Sequence
  Synthetic Construct
ttgaaccccataacatttcaacgaattcctcatcctttctgtgaatcaagagcctgaaaagaaatggtgaaataatatgatcctctcttctttgaaagctcaaagctatgttggaccagaagtaaagtgttctcgtttctatttaataacttgaaaggttccgaggggccattgaggaaactcctcccttttaatatcaatgtgtatttattgcaaaaataatgtagcatcgagtggtattttatagcttatccaaaaacctcctgggtttaacgcattgtgatagtcccgttttcttctcagcccaggtcctatgcatcctcatctatgcagggctgttatctgcatataattttttttttttttaagacaaagtcttgctctgtcgccccggctggagtgcagtggtgcaatctcggctcactgcaacctccgcctcccaggttcaagcggttcttccgcctcagcctaccgagtagctgggactacaggcatgcgccaccacacctaggtgatttttgtatttttagtagagacaggggtttcaccatgttgaccaggctggtctcgaactcctgatctcaagcgatccacccgcctcagcctcccaaagtgctgggattacaggcataagccactacgcccggcctcaattttgtattgtactttttctttctttctttaatagagacagggtctcactatgttgactaggttggtctagaactcctgggcacaagctgtccgcccgcttctgcctcccaaagtgctgggattgcaggcgtgaaccaccgcccctggctacaggtgccttcttgtctcaatttgcctttgacctttcttagggacttgttttctgcttttcctgctctttgtccgctgatctcctgggaagaaagcttccgaaaaggacaccgtttcaggggcgagtgacgccggggtgcccaggccgcgccccagttccgggtttgcacccggtcttcttgccctgccccgcccg

SEQ ID NO: 494 - DNA, 20 bp, Artificial Sequence
  Synthetic Construct
aaggcgcttgagtatgcctg

SEQ ID NO: 495 - DNA, 20 bp, Artificial Sequence
  Synthetic Construct
gtgggcttccactcctatcg

SEQ ID NO: 496 - DNA, 21 bp, Artificial Sequence
  Synthetic Construct
atggacaggactgaacgtctt

SEQ ID NO: 497 - DNA, 20 bp, Artificial Sequence
  Synthetic Construct
tccagcaggtcagcaaagaa

SEQ ID NO: 498 - DNA, 20 bp, Artificial Sequence
  Synthetic Construct
ataatcccaagcggtttgct

SEQ ID NO: 499 - DNA, 20 bp, Artificial Sequence
  Synthetic Construct
ctgccagtctggactgttct

SEQ ID NO: 500 - DNA, 74 bp, Artificial Sequence
  Synthetic Construct
tctcttctttgaaagctcaaagctatgttggaccagaagtaaagtgttctcgtttctatttaataacttgaaag

SEQ ID NO: 501 - DNA, 36 bp, Artificial Sequence
  Synthetic Construct
gttttcttctcagcccaggtcctatgcatcctcatc

SEQ ID NO: 502 - DNA, 31 bp, Artificial Sequence
  Synthetic Construct
tacaggtgccttcttgtctcaatttgccttt

SEQ ID NO: 503 - DNA, 124 bp, Artificial Sequence
  Synthetic Construct
ttgttttctgcttttcctgctctttgtccgctgatctcctgggaagaaagcttccgaaaaggacaccgtttcaggggcgagtgacgccggggtgcccaggccgcgccccagttccgggtttgca

SEQ ID NO: 504 - DNA, 125 bp, Artificial Sequence
  Synthetic Construct
aggttccgaggggccattgaggaaactcctcccttttaatatcaatgtgtatttattgcaaaaataatgtagcatcgagtggtattttatagcttatccaaaaacctcctgggtttaacgcattg

SEQ ID NO: 505 - DNA, 21 bp, Artificial Sequence
  Synthetic Construct
  misc_feature (20)..(21): deoxythymidine (dT) base
acuacugagugacaguagatt

SEQ ID NO: 506 - DNA, 21 bp, Artificial Sequence
  Synthetic Construct
  misc_feature (20)..(21): deoxythymidine (dT) base
ucuacugucacucaguagutt

SEQ ID NO: 507 - DNA, 21 bp, Artificial Sequence
  Synthetic Construct
  misc_feature (20)..(21): deoxythymidine (dT) base
gaaguucagagccgcucaatt

SEQ ID NO: 508 - DNA, 21 bp, Artificial Sequence
  Synthetic Construct
  misc_feature (20)..(21): deoxythymidine (dT) base
uugagcggcucugaacuuctt

(19) United States
(12) Patent Application Publication
(10) Pub. No.: US 2022/0096516 A1
(43) Pub. Date: Mar. 31, 2022

(54) OLIGONUCLEOTIDE MOLECULE AND APPLICATION THEREOF IN TUMOR THERAPY

(71) Applicant: Ractigen Therapeutics, Nantong City, Jiangsu (CN)

(72) Inventors: Tao KANG, Nantong City, Jiangsu (CN); Longcheng LI, Nantong City, Jiangsu (CN); Moorim KANG, Nantong City, Jiangsu (CN)

(21) Appl. No.: 17/427,393

(22) PCT Filed: Jun. 25, 2019
(86) PCT No.: PCT/CN2019/092720

§ 371 Date: Jul. 30, 2021

(30) Foreign Application Priority Data
Jan. 30, 2019 (CN) 201910092375.8

(51) Int. Cl. A61K 31/7088; A61P 35/00

(52) U.S. Cl. CPC A61K 31/7088; A61P 35/00

(57) ABSTRACT

The present invention relates to oligomeric nucleic acids and uses thereof for the treatment of tumors. The oligomeric nucleic acid for tumor treatment provided by the present application can be small activating nucleic acid molecules. A small activating nucleic acid molecule of the present invention can be a double-stranded or single-stranded RNA molecule targeting the promoter region of an LHPP gene comprising a first nucleic acid strand and a second nucleic acid strand. The double-stranded RNA molecule targeting the promoter region of the LHPP gene comprises two nucleic acid strands of 16 to 35 nucleotides in length, wherein one of the nucleic acid strands has at least 75% homology or complementarity to a target selected from the promoter region of the LHPP gene. The present invention also relates to pharmaceutical compositions comprising the small activating nucleic acids and optional pharmaceutically acceptable carriers, and methods for upregulating the expression of the LHPP gene in a cell and methods for treating diseases or conditions related to insufficient or decreased expression of LHPP gene by using the small activating nucleic acid molecules or the pharmaceutical compositions.

### TECHNICAL FIELD

The present invention belongs to the technical field of nucleic acids, and more particularly it relates to oligomeric nucleic acids associated with gene activation, e.g., small activating nucleic acid molecules, uses of the small activating nucleic acid molecules in activating/up-regulating the transcription of histidine phosphatase LHPP (Phospholysine phosphohistidine inorganic pyrophosphate phosphatase) gene, and uses thereof in treating LHPP deficiency-associated diseases, such as tumors.

### BACKGROUND

The histidine phosphatase LHPP (Phospholysine phosphohistidine inorganic pyrophosphate phosphatase) gene on the chromosome 10q26.13 of the human genome has seven exons, and can be transcribed to produce nine LHPP splice variants. LHPP is expressed in various human tissues, including liver, kidney, brain tissues, and the like (1-3).

Multiple genome-wide association studies (GWAS) have showed that LHPP is a risk factor in causing severe depression (2; 4-7) and cancer (8-10). A recent study on hepatocellular carcinoma (HCC) showed that LHPP was proved to be histidine phosphatase with a tumor suppressor function (11). Using a hepatocellular carcinoma mouse model (mTOR-driven), this study showed an significant increase in histidine phosphorylation levels in the tumor tissues during the tumor formation. A proteome analysis showed increased expression levels of histidine kinase NME1 (Nucleoside diphosphate kinase A) and histidine kinase NME2 (Nucleoside diphosphate kinase B) in tumor tissue, with decreased expression level of histidine phosphatase LHPP. Interestingly, after the LHPP gene was introduced into the hepatocellular carcinoma mouse model, tumor weight was reduced and liver function was protected. Furthermore according to an analysis of LHPP expression in a liver cancer patient sample, LHPP expression was negatively correlated with tumor severity and positively correlated with the overall survival rate of the patient (11). Therefore, LHPP has the potential as a therapeutic target for hepatocellular carcinoma.

China is a country with the prevalence of liver cancer and more than half of the cases of HCC worldwide occur in China (12). However, there are few approaches to treat liver cancer and a limited curative effect exists. Therefore, there is an urgent need to develop an innovative drug. The present invention provides a highly specific, small activating RNA capable of continuously activating/upregulating LHPP transcription, which can in turn increase LHPP protein expression and have a remarkable tumor inhibition effect as shown by in in-vitro and in-vivo studies. The small activating RNA is expected to become an effective treatment for cancers.

### SUMMARY

### Disclosure

One objective of the present invention is to provide small activating nucleic acid molecules, which can increase the expression of LHPP protein by activating/up-regulating the transcription of the LHPP gene via an RNA activation process to thereby treat an LHPP deficiency-associated disease, such as tumors.

Another objective of the present invention is to provide compositions and formulations comprising the small activating nucleic acid molecules with the tumor inhibitory activity.

Yet another objective of the present invention is to provide a use of the small activating nucleic acid molecule with the tumor inhibitory activity or the compositions or formulations comprising the same in preparing a medicament for activating/up-regulating the expression of the LHPP gene in a cell.

Still yet another objective of the present invention is to provide a method for activating/up-regulating the expression of LHPP gene in a cell.

Still another objective of the present invention is to provide the use of the small activating nucleic acid molecule with the tumor inhibitory activity or the compositions or formulations comprising the same in preparing a therapeutic for treating a disease or condition, e.g., tumors, which is related to LHPP deficiency or insufficiency, or a method for treating a disease or condition, e.g., tumors, related to LHPP deficiency or insufficiency.

Another objective of the present invention is to provide an isolated target site of a small activating nucleic acid molecule on an LHPP gene, wherein the target site comprises or is selected from any sequence of continuous 16 to 35 nucleotides in any of the sequences set forth in SEQ ID NOs: 500-504.

### Technical Solution

In one aspect of the present invention, a small activating nucleic acid molecule is provided, which can activate/up-regulate the expression of LHPP gene in a cell. One strand of the small activating nucleic acid molecule has at least 75% homology or complementarity to a nucleic acid sequence of 16 to 35 nucleotides in length in a promoter region of LHPP gene, thereby activating or up-regulating the expression of the gene, wherein the promoter region comprises 1000 nucleotides upstream of a transcription start site. Specifically, one strand of the small activating nucleic acid molecule comprises or is selected from a nucleic acid sequence having at least 75%, e.g., at least about 79%, about 80%, about 85%, about 90%, about 95%, about 99% or 100%, homology or complementarity to a sequence of continuous 16 to 35 nucleotides in positions −917 to −844 (H1, SEQ ID NO: 500), positions −710 to −675 (H2, SEQ ID NO: 501), positions −198 to −168 (H3, SEQ ID NO: 502), positions −151 to −28 (H4, SEQ ID NO: 503) or positions −845 to −711 (H5, SEQ ID NO: 504) upstream of the transcription start site in the LHPP gene promoter. More specifically, one strand of the small activating nucleic acid molecule of the present invention has at least 75%, e.g., at least about 79%, about 80%, about 85%, about 90%, about 95%, about 99% or about 100%, homology or complementarity to any nucleotide sequence selected from SEQ ID NOs: 329-492. In one specific embodiment, one strand of the small activating nucleic acid molecule of the present invention comprises a nucleic acid sequence having at least 75%, e.g., at least about 79%, about 80%, about 85%, about 90%, about 95%, about 99% or about 100% homology or complementarity to any nucleotide sequence selected from SEQ ID NOs: 329-492. In another embodiment, one strand of the small activating nucleic acid molecule of the present invention consists of a nucleic acid sequence having at least 75%, e.g., at least about 79%, about 80%, about 85%, about 90%, about 95%, about 99% or about 100%, homology or complementarity to any nucleotide sequence selected from SEQ ID NOs: 329-492.

The small activating nucleic acid molecule of the present invention comprises a double-stranded small activating nucleic acid molecule targeting the promoter region of LHPP gene comprising a first nucleic acid strand and a second nucleic acid strand, wherein the first nucleic acid strand has at least 75% homology or complementarity to any sequence of 16 to 35 continuous nucleotides in positions −917 to −844 (SEQ ID NO: 500), positions −710 to −675 (SEQ ID NO: 501), positions −198 to −168 (SEQ ID NO: 502), positions −151 to −28 (SEQ ID NO: 503) or positions −845 to −711 (SEQ ID NO: 504) upstream of the transcription start site in the LHPP gene promoter, and the first nucleic acid strand and the second nucleic acid strand can complementarily form a double-stranded nucleic acid structure capable of activating the expression of LHPP gene in a cell.

The first nucleic acid strand and the second nucleic acid strand of the small activating nucleic acid molecule of the present invention can be present either on two different nucleic acid strands or on the same nucleic acid strand. When the first nucleic acid strand and the second nucleic acid strand are located on two different strands, at least one strand of the small activating nucleic acid molecule can have overhangs at the 5′ terminus and/or the 3′ terminus, e.g. overhangs of 0 to 6 nucleotides in length at 3′ terminus, such as overhangs of 0, 1, 2, 3, 4, 5 or 6 nucleotides in length. Preferably, both strands of the small activating nucleic acid molecule of the present invention have overhangs; more preferably, the 3′ terminus of both strands of the small activating nucleic acid molecule can have overhangs of 0 to 6 nucleotides in length, e.g., overhangs of 0, 1, 2, 3, 4, 5 or 6 nucleotides in length; and most preferably overhangs of 2 or 3 nucleotides in length. Preferably, the nucleotide of the overhang can be dT.

The small activating nucleic acid molecule of the present invention can also comprise a small activating nucleic acid molecule capable of forming a double-stranded region hairpin structure, e.g., a single-stranded small activating RNA molecule. In one embodiment, the small activating nucleic acid molecule of the present invention comprises a single-stranded small activating RNA molecule targeting the promoter region of LHPP gene, wherein the single-stranded small activating nucleic acid molecule can form a double-stranded region hairpin structure. When the first nucleic acid strand and the second nucleic acid strand are present on the same nucleic acid strand, preferably, the small activating nucleic acid molecule of the present invention can be a hairpin single-stranded nucleic acid molecule, wherein the first nucleic acid strand and the second nucleic acid strand have complementary regions capable of forming a double-stranded nucleic acid structure, and the double-stranded nucleic acid structure can promote the expression of LHPP gene in a cell with, for example, a RNA activation mechanism.

In the aforementioned small activating nucleic acid molecule, the first nucleic acid strand and the second nucleic acid strand can have 16 to 35 nucleotides, e.g., 16, 17, 18, 19, 20, 21, 22, 23, 24, 25, 26, 27, 28, 29, 30, 31, 32, 33, 34 or 35 nucleotides in length.

In one embodiment, the first nucleic acid strand of the small activating nucleic acid molecule of the present invention has at least 75%, e.g., at least about 79%, about 80%, about 85%, about 90%, about 95%, about 99% or about 100%, identity or homology to any nucleotide sequence selected from SEQ ID NOs: 1-164, and the second nucleic acid strand of the small activating nucleic acid molecule has at least 75%, e.g., at least about 79%, about 80%, about 85%, about 90%, about 95%, about 99% or about 100%, identity or homology to any nucleotide sequence selected from SEQ ID NOs: 165-328. In one embodiment, the first nucleic acid strand of the small activating nucleic acid molecule of the present invention comprises a nucleic acid sequence having at least 75%, e.g., at least about 79%, about 80%, about 85%, about 90%, about 95%, about 99% or about 100%, identity or homology to any nucleotide sequence selected from SEQ ID NOs: 1-164, or consists of a nucleic acid sequence having at least 75%, e.g., at least about 79%, about 80%, about 85%, about 90%, about 95%, about 99% or about 100%, identity or homology to any nucleotide sequence selected from SEQ ID NOs: 1-164; and the second nucleic acid strand of the small activating nucleic acid molecule of the present invention comprises a nucleic acid sequence having at least 75%, e.g., at least about 79%, about 80%, about 85%, about 90%, about 95%, about 99% or about 100%, identity or homology to any nucleotide sequence selected from SEQ ID NOs: 165-328, or consists of a nucleic acid sequence having at least 75%, e.g., at least about 79%, about 80%, about 85%, about 90%, about 95%, about 99% or about 100%, identity or homology to any nucleotide sequence selected from SEQ ID NOs: 165-328. In a specific embodiment, the first nucleic acid strand of the small activating nucleic acid molecule of the present invention can comprise or be selected from any nucleotide sequence set forth in SEQ ID NOs: 1-164, and the second strand can comprise or be selected from any nucleotide sequence set forth in SEQ ID NOs: 165-328. In one embodiment, the small activating nucleic acid molecule described herein can be synthesized, transcribed in vitro or expressed by a vector.

All the nucleotides in the small activating nucleic acid molecule described herein can be natural non-chemically modified nucleotides or can comprise at least one modification. In one embodiment, the modification in the small activating nucleic acid molecule described herein can be chemical modification, for example, at least one nucleotide can have chemical modification, and the chemical modification used in the present invention can comprise or be selected from one or more or any combination of the following modifications:

(1) modification of a phosphodiester bond of nucleotides in the nucleotide sequence of the small activating nucleic acid molecule;

(2) modification of 2′-OH of a ribose in the nucleotide sequence of the small activating nucleic acid molecule; and

(3) modification of a base in the nucleotide sequence of the small activating nucleic acid molecule;

(4) at least one nucleotide in the nucleotide sequence of the small activating nucleic acid molecule being a locked nucleic acid.

The chemical modification described herein is well-known to those skilled in the art, and the modification of the phosphodiester bond refers to the modification of oxygen in the phosphodiester bond including, but not limited to phosphorothioate modification and boranophosphate modification. Both modifications can stabilize an saRNA structure and maintain high specificity and high affinity for base pairing.

The ribose modification refers to the modification of 2′-OH in pentose of a nucleotide, i.e., the introduction of some substituents into hydroxyl positions of the ribose, for example, including, but not limited to 2′-fluoro modification, 2′-oxymethyl modification, 2′-oxyethylidene methoxy modification, 2,4′-dinitrophenol modification, locked nucleic acid (LNA), 2′-amino modification, 2′-deoxy modification, etc.

The base modification refers to the modification of the base of a nucleotide, for example, including, but not limited to 5′-bromouracil modification, 5′-iodouracil modification, N-methyluracil modification, 2,6-diaminopurine modification, etc.

These modifications can increase the bioavailability of the small activating nucleic acid molecule, improve affinity to a target sequence, and enhance resistance to nuclease hydrolysis in a cell.

In addition, in order to promote the access of the small activating nucleic acid molecule into a cell, on the basis of the aforementioned modifications, a lipophilic group (such as cholesterol) can be introduced into the terminus of the first nucleic acid strand or the second nucleic acid strand of the small activating nucleic acid molecule to facilitate the interaction with the gene promoter region in the cell nucleus through the cell membrane and nuclear membrane composed of lipid bilayers.

After contacting a cell, the small activating nucleic acid molecule provided by the present invention can effectively activate or up-regulate the expression of the LHPP gene in the cell, preferably up-regulate the expression by at least 10%.

Another aspect of the present invention relates to a nucleic acid coding the small activating nucleic acid molecule described herein. In one embodiment, the nucleic acid can be a DNA molecule.

Another aspect of the present invention provides a cell comprising the aforementioned small activating nucleic acid molecule or the nucleic acid coding the small activating nucleic acid molecule described herein. In one embodiment, the small activating nucleic acid molecule of the present invention can be a double-stranded small activating nucleic acid molecule targeting the promoter region of LHPP gene, comprising a first nucleic acid strand and a second nucleic acid strand. In another embodiment, the small activating nucleic acid molecule of the present invention can be a single-stranded small activating nucleic acid molecule targeting the promoter region of LHPP gene.

Another aspect of the present invention provides a composition, e.g., a pharmaceutical composition. The composition comprises the small activating nucleic acid molecule of the present invention or the nucleic acid coding the small activating nucleic acid molecule described herein and optionally, a pharmaceutically acceptable carrier. In one embodiment, the pharmaceutically acceptable carrier can comprise or be chosen from a liposome, a high-molecular polymer, and a polypeptide. In one embodiment, the composition of the present invention contains 1 nM to 150 nM, e.g., 1 nM to 100 nM, such as 1 nM to 50 nM, e.g., 10 nM, 20 nM, 30 nM, 40 nM, 50 nM, 60 nM, 70 nM, 80 nM, 90 nM, 100 nM, 110 nM, 120 nM, 130 nM, 140 nM or 150 nM of small activating nucleic acid molecule of the present invention. In another embodiment, the composition of the present invention can further comprise other compounds, such as a low-molecular-weight compound for tumor treatment. In one embodiment, the low-molecular-weight compound for tumor treatment can comprise multi-target anti-tumor drugs (e.g., Sorafenib (targets: PDGFR, KIT and RAF) (SELLECK, S1040)), tyrosine kinase inhibitors (e.g., Lenvatinib (targets: FGFR, VEGFR2, PDGFR and KIT) (SELLECK, S1164)), kinase inhibitors (e.g., Regorafenib (targets: FGFR, VEGFR2, PDGFR, KIT and RAF) (SELLECK, S1178)), or Cabozantini (inhibiting MET, VEGFR2 and RET signal transduction) (SELLECK, S1119).

In another aspect of the present invention, a kit is provided, which comprises the aforementioned small activating nucleic acid molecule, the nucleic acid coding the small activating nucleic acid molecule described herein, the cell comprising the small activating nucleic acid molecule of the present invention or the nucleic acid coding the small activating nucleic acid molecule of the present invention, or the composition comprising the small activating nucleic acid molecule of the present invention.

Another aspect of the present invention relates to a use of the small activating nucleic acid molecule of the present invention, the nucleic acid coding the small activating nucleic acid molecule of the present invention, the cell comprising the small activating nucleic acid molecule of the present invention and the nucleic acid coding the small activating nucleic acid molecule of the present invention or the composition comprising the small activating nucleic acid molecule of the present invention in preparing a drug or formulation for activating/up-regulating the expression of LHPP gene in a cell.

Another aspect of the present invention also relates to a method for activating/up-regulating the expression of LHPP gene in a cell. The method comprises administering the small activating nucleic acid molecule of the present invention, the nucleic acid coding the small activating nucleic acid molecule of the present invention or the composition comprising the small activating nucleic acid molecule of the present invention to the cell.

The small activating nucleic acid molecule of the present invention can be directly introduced into a cell or it can be produced in the cell after a nucleotide sequence coding the small activating nucleic acid molecule of the present invention is introduced into the cell. The cell is preferably a mammalian cell, more preferably a human cell. The aforementioned cell can be in vitro, such as a cell line or a cell strain, or can be present in a mammalian body, such as a human body. The human body can be a patient suffering from a disease or condition related to insufficient or decreased expression of LHPP protein. The small activating nucleic acid molecule of the present invention can be administered at a sufficient dose to treat the disease or condition related to a deficiency in the amount of LHPP protein or insufficient or decreased expression of LHPP protein. Specifically, the disease or condition related to a deficiency in the amount of LHPP protein or insufficient or decreased expression of LHPP protein can comprise, for example, tumors, e.g., solid tumors, and the solid tumors can comprise, for example, liver cancer, lung cancer, bladder cancer, prostatic cancer, glioma, etc.

Another aspect of the present invention provides an isolated small activating nucleic acid molecule targeting site of an LHPP gene, which has any sequence of 16 to 35 continuous nucleotides in the promoter region of the LHPP gene, and preferably, the acting site comprises or is chosen from any sequence of 16 to 35 continuous nucleotides in any of the sequences set forth in SEQ ID NOs: 500-504. Specifically, the targeting site can comprise or be chosen from any nucleotide sequence set forth in SEQ ID NOs: 329-492.

Another aspect of the present invention relates to a method for treating a disease or condition related to insufficient or decreased expression of LHPP protein in a subject, which comprises administering the small activating nucleic acid molecule of the present invention, the nucleic acid coding the small activating nucleic acid molecule of the present invention, the cell comprising the small activating nucleic acid molecule of the present invention or the nucleic acid coding the small activating nucleic acid molecule of the present invention or the composition comprising the small activating nucleic acid molecule of the present invention at a therapeutically effective amount to the subject. In one embodiment, the method for treating a disease or condition related to insufficient or decreased expression of LHPP protein in a subject in the present invention comprises administering the small activating nucleic acid molecule of the present invention, the nucleic acid coding the small activating nucleic acid molecule of the present invention, the cell comprising the small activating nucleic acid molecule of the present invention or the nucleic acid coding the small activating nucleic acid molecule of the present invention or the composition comprising the small activating nucleic acid molecule of the present invention and a low-molecular-weight compound, antibody, polypeptide or protein at therapeutically effective amounts to the subject. The subject may be a mammal, such as a human. In one embodiment, the disease or condition related to insufficient or decreased expression of LHPP protein may comprise, for example, tumors, e.g., solid tumors, and the solid tumors may comprise, for example, liver cancer, lung cancer, bladder cancer, prostatic cancer, glioma, etc.

Another aspect of the present invention relates to a use of the small activating nucleic acid molecule of the present invention, the nucleic acid coding the small activating nucleic acid molecule of the present invention, the cell comprising the small activating nucleic acid molecule of the present invention or the nucleic acid coding the small activating nucleic acid molecule of the present invention or the composition comprising the small activating nucleic acid molecule of the present invention in preparing a drug for treating a disease or condition related to insufficient or decreased expression of LHPP protein. The subject may be a mammal, such as a human. In one embodiment, the disease related to insufficient or decreased expression of LHPP protein may comprise, for example, tumors, e.g., solid tumors, and the solid tumors may comprise, for example, liver cancer, lung cancer, bladder cancer, prostatic cancer, glioma, etc.

In one embodiment, provided is a use of the small activating nucleic acid molecule of the present invention, the nucleic acid coding the small activating nucleic acid molecule of the present invention, the cell comprising the small activating nucleic acid molecule of the present invention or the nucleic acid coding the small activating nucleic acid molecule of the present invention or the composition comprising the small activating nucleic acid molecule of the present invention in preparing a drug for treating tumors, such as solid tumors, wherein the solid tumors can comprise, for example, liver cancer, lung cancer, bladder cancer, prostatic cancer, glioma, etc.

Another aspect of the present invention relates to a use of the small activating nucleic acid molecule of the present invention, the nucleic acid coding the small activating nucleic acid molecule of the present invention, the cell comprising the small activating nucleic acid molecule of the present invention or the nucleic acid coding the small activating nucleic acid molecule of the present invention or the composition comprising the small activating nucleic acid molecule of the present invention and a chemotherapeutic agent, radiotherapy, cell therapy, micromolecule, polypeptide, protein, antibody or other anti-tumor drugs in preparing a drug or pharmaceutical composition for treating a disease or condition related to insufficient or decreased expression of LHPP protein.

In one embodiment, provided is a use of the small activating nucleic acid molecule of the present invention, the nucleic acid coding the small activating nucleic acid molecule of the present invention, the cell comprising the small activating nucleic acid molecule of the present invention or the nucleic acid coding the small activating nucleic acid molecule of the present invention or the composition comprising the small activating nucleic acid molecule of the present invention and a chemotherapeutic agent, radiotherapy, cell therapy, micromolecule, polypeptide, protein, antibody or other anti-tumor drugs in preparing a drug or pharmaceutical composition for treating tumors, such as solid tumors, wherein the solid tumors can comprise, for example, liver cancer, lung cancer, bladder cancer, prostatic cancer, glioma, etc. In one embodiment, the chemotherapeutic agent comprises or is chosen from Sorafenib, Lenvatinib, Regorafenib and Cabozantinib.

Further embodiments include methods and uses of the small activating nucleic acid molecule of the present invention in combination with a chemotherapeutic agent, radiotherapy, cell therapy, micromolecule, polypeptide, protein, antibody, or other anti-tumor drugs, and the chemotherapeutic agent is preferably selected from Sorafenib, Lenvatinib, Regorafenib and Cabozantinib.

Advantages of the Present Invention

The small activating nucleic acid molecule capable of activating/up-regulating the expression of LHPP gene provided by the present invention can permanently activate LHPP gene, therefore efficiently and specifically up-regulating or restoring the expression of LHPP gene and protein while featuring lower toxic and side effects, and it can be used in preparing a drug or formulation for a disease or symptom related to insufficient or decreased expression of LHPP protein. Moreover, the activating saRNA of LHPP shows a good synergistic effect when used in combination with other anti-tumor drugs, reflecting a synergistic effect in terms of anti-tumor effect.

### BRIEF DESCRIPTION OF THE DRAWINGS

FIG. 1 is a schematic of LHPP gene. The drawing shows the LHPP gene structure and a 1 kb promoter region used for designing the saRNA, in which a 449 bp Alu repeat sequence is excluded.

FIG. 2 shows changes in the expression levels of LHPP mRNA mediated by saRNA. 290 LHPP promoter-targeting saRNAs were individually transfected into Huh7 cells, and 72 hours later, the expression level of LHPP mRNA was analyzed by one-step RT-qPCR. The drawing shows changes in the expression level of LHPP caused by each saRNA relative to a control treatment (control, mock) ordered by the target positions of the saRNA in the promoter region from −917 to −28.

FIG. 3 shows saRNA hot spot regions in LHPP promoter. 290 LHPP saRNAs promoter-targeting saRNAs were individually transfected into Huh7 cells, and 72 hours later, the expression level of LHPP mRNA was analyzed by one-step RT-qPCR. The drawing shows changes in the expression level of LHPP mRNA caused by each saRNA relative to a control treatment (control, mock) ordered by the target positions of the saRNA in the promoter region from −917 to −28. The numbers above or below indicate the boundaries of the hot spot regions (relative to the LHPP transcription start site (TSS)).

FIG. 4 shows a negative correlation between expression levels of LHPP mRNA and cell viability. 290 LHPP promoter-targeting saRNAs were individually transfected into Huh7 cells, and 72 hours later, one-step RT-qPCR was employed to analyze the expression level of LHPP mRNA and cell viability was detected by the CCK-8 method. The thin line represents relative LHPP mRNA expression level (log 2), and the thick line represents cell viability.

FIG. 5 shows saRNAs inducing the expression of LHPP and inhibiting AKT phosphorylation. 10 LHPP promoter-targeting saRNAs were individually transfected into Huh7 cells and were analyzed 72 hours later. In FIG. 5A, the expression levels of LHPP mRNA was analyzed by RT-qPCR. (In FIG. 5B, the protein levels of LHPP, pAKT and AKT were detected by a Western blot method.

FIG. 6 shows saRNAs inducing the mRNA expression of LHPP and inhibiting the proliferation of liver cancer cells. 8 LHPP promoter-targeting saRNAs were individually transfected into liver cancer cells at 10 nM for 72 h. In FIG. 6A, the expression level of LHPP mRNA was analyzed by RT-qPCR. In FIG. 6B, cell viability in the saRNA treatment group was evaluated by the CCK-8 method and recorded as a percentage relative to the viability of cells in a control (Mock) treatment group.

FIG. 7 shows saRNAs inducing the expression levels of LHPP mRNA and inhibiting the proliferation of several other cancer cells. 8 LHPP promoter-targeting saRNAs were individually transfected into cancer cells at 10 nM for 72 h. In FIG. 7A, the expression level of LHPP mRNA was analyzed by RT-qPCR. In FIG. 7B, cell viability in the saRNA treatment group was evaluated by the CCK-8 method and recorded as a percentage relative to the viability of cells in a control (Mock) treatment group.

FIG. 8 shows saRNA inhibiting the proliferation of HepG2 cells in combination with chemical drugs. The saRNAs were transfected into cancer cells at different concentrations in combination with a panel of chemical drugs. In FIG. 8A, cell viability in the saRNA treatment group was evaluated by the CCK-8 method and recorded as a percentage relative to the viability of cells in a control (Mock) treatment group. In FIG. 8B, Compusyn© version 1.0 software was used to draw a combination index graphs; and to calculate combination index values as shown in FIG. 8C.

FIG. 9 shows saRNA inhibiting the proliferation of U87MG cells in combination with chemical drugs. The sRNAs were transfected into cancer cells at different concentrations in combination with different chemical drugs. In FIG. 9A, cell viability in the saRNA treatment group was evaluated by the CCK-8 method and recorded as a percentage relative to the viability of cells in a control (Mock) treatment group. In FIG. 9B, Compusyn© version 1.0 software was used to draw a combination index graphs; and to calculate combination index values as shown in FIG. 9C.

FIG. 10 shows saRNA inhibiting the growth of a HepG2 transplanted tumor in combination with a chemical drug. The small activating RNA shown was injected into a tumor at a dose of 1 mg/kg in combination with a chemical drug. The volume change of the transplanted tumor was recorded during administration.

FIG. 11 shows saRNA inhibiting the growth of a U87MG transplanted tumor in combination with a chemical drug. The saRNA was injected into a tumor at a dose of 1 mg/kg in combination with a chemical drug, and the volume change of the transplanted tumor was recorded during administration.

### DETAILED DESCRIPTION

In the present invention, the related terms are defined as follows:

The term “complementarity” as used herein refers to the capability of forming base pairs between two oligonucleotide strands. The base pairs are generally formed through hydrogen bonds between nucleotides in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing).

Complementarity includes complete complementarity and incomplete complementarity. “Complete complementarity” or “100% complementarity” means that each nucleotide from the first oligonucleotide strand can form a hydrogen bond with a nucleotide at a corresponding position in the second oligonucleotide strand in the double-stranded region of the double-stranded oligonucleotide molecule without “mispairing”. “Incomplete complementarity” means that not all the nucleotide units of the two strands are bonded with each other by hydrogen bonds. For example, for two oligonucleotide strands each of 20 nucleotides in length in the double-stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. Substantial complementarity refers to at least about 75%, about 79%, about 80%, about 85%, about 90%, about 95%, about 99% or about 100% complementarity.

The term “oligonucleotide” as used herein refers to polymers of nucleotides, and includes, but is not limited to, single-stranded or double-stranded molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides. The oligonucleotide for activating target gene transcription described herein is a small activating nucleic acid molecule.

The terms “oligonucleotide strand” and “oligonucleotide sequence” as used herein can be used interchangeably, referring to a generic term for short nucleotide sequences having less than 35 bases (including nucleotides in deoxyribonucleic acid (DNA) or ribonucleic acid (RNA)). In the present invention, an oligonucleotide strand can have any of 16 to 35 nucleotides in length.

As used herein, the term “first nucleic acid strand” can be a sense strand or an antisense strand. The sense strand of a small activating RNA refers to a nucleic acid strand contained in a small activating RNA duplex which has identity to the coding strand of the promoter DNA sequence of a target gene, and the antisense strand refers to a nucleic acid strand in the small activating RNA duplex which is complementary with the sense strand.

As used herein, the term “second nucleic acid strand” can also be a sense strand or an antisense strand. If the first oligonucleotide strand is a sense strand, the second oligonucleotide strand is an antisense strand; and if the first oligonucleotide strand is an antisense strand, the second oligonucleotide strand is a sense strand.

The term “gene” as used herein refers to all nucleotide sequences required to encode a polypeptide chain or to transcribe a functional RNA. “Gene” can be an endogenous or fully or partially recombinant gene for a host cell (for example, because an exogenous oligonucleotide and a coding sequence for encoding a promoter are introduced into a host cell, or a heterogeneous promoter adjacent to an endogenous coding sequence is introduced into a host cell). For example, the term “gene” comprises a nucleic acid sequence consisting of exons and introns. Protein-coding sequences are, for example, sequences contained within exons in an open reading frame between an initiation codon and a termination codon, and as used herein, “gene” can comprise such as a gene regulatory sequence, such as a promoter, an enhancer, and all other sequences known in the art for controlling the transcription, expression or activity of another gene, no matter whether the gene comprises a coding sequence or a non-coding sequence. In one case, for example, “gene” can be used to describe a functional nucleic acid comprising a regulatory sequence such as a promoter or an enhancer. The expression of a recombinant gene can be controlled by one or more types of heterogeneous regulatory sequences.

The term “target gene” as used herein can refer to nucleic acid sequences naturally present in organisms, transgenes, viral or bacterial sequences, can be chromosomes or extrachromosomal genes, and/or can be transiently or stably transfected or incorporated into cells and/or chromatins thereof. The target gene can be a protein-coding gene or a non-protein-coding gene (such as a microRNA gene and a long non-coding RNA gene). The target gene generally contains a promoter sequence, and the positive regulation for the target gene can be achieved by designing a small activating nucleic acid molecule having sequence identity (also called homology) to the promoter sequence, characterized as the up-regulation of expression of the target gene. “Sequence of a target gene promoter” refers to a non-coding sequence of the target gene, and the reference of the sequence of a target gene promoter in the phrase “complementary with the sequence of a target gene promoter” of the present invention refers to a coding strand of the sequence, also known as a non-template strand, i.e., a nucleic acid sequence having the same sequence as the coding sequence of the gene. “Target” or “target sequence” refers to a sequence fragment in the sequence of a target gene promoter which is homologous or complementary with a sense oligonucleotide strand or an antisense oligonucleotide strand of a small activating nucleic acid molecule.

As used herein, the terms “sense strand” and “sense nucleic acid strand” can be used interchangeably, and the sense oligonucleotide strand of a small activating nucleic acid molecule refers to the first nucleic acid strand having sequence identity to the coding strand of the sequence of a target gene promoter in the small activating nucleic acid molecule duplex.

As used herein, the terms “antisense strand” and “antisense nucleic acid strand” can be used interchangeably, and the antisense oligonucleotide strand of a small activating nucleic acid molecule refers to the second nucleic acid strand which is complementary with the sense oligonucleotide strand in the small activating nucleic acid molecule duplex.

The term “coding strand” as used herein refers to a DNA strand in the target gene which cannot be used for transcription, and the nucleotide sequence of this strand is the same as that of a RNA produced from transcription (in the RNA, T in DNA is replaced by U). The coding strand of the double-stranded DNA sequence of the target gene promoter described herein refers to a promoter sequence on the same DNA strand as the DNA coding strand of the target gene.

The term “template strand” as used herein refers to the other strand complementary with the coding strand in the double-stranded DNA of the target gene, i.e., the strand that, as a template, can be transcribed into RNA, and this strand is complementary with the transcribed RNA (A to U and G to C). In the process of transcription, RNA polymerase binds to the template strand, moves along the 3′→5′ direction of the template strand, and catalyzes the synthesis of the RNA along the 5′→3′ direction. The template strand of the double-stranded DNA sequence of the target gene promoter described herein refers to a promoter sequence on the same DNA strand as the DNA template strand of the target gene.

The term “promoter” as used herein refers to a sequence which plays a regulatory role for the transcription of a protein-coding or RNA-coding nucleic acid sequence by spacially associating with the coding sequence. Generally, a eukaryotic gene promoter contains 100 to 5000 base pairs, although this length range is not intended to limit the term “promoter” as used herein. Although the promoter sequence is generally located at the 5′ terminus of a protein-coding or RNA-coding sequence, it can also exist in exon and intron sequences.

The term “transcription start site” as used herein refers to a nucleotide marking the transcription start on the template strand of a gene. The transcription start site can appear on the template strand of the promoter region. A gene can have more than one transcription start site.

The term “identity” or “homology” as used herein means that one oligonucleotide strand (sense or antisense strand) of an small activating RNA has sequence similarity with a coding strand or a template strand in a region of the promoter sequence of a target gene. As used herein, the “identity” or “homology” can be at least about 75%, about 79%, about 80%, about 85%, about 90%, about 95%, about 99% or about 100%.

The term “overhang” as used herein refers to non-base-paired nucleotides at the terminus (5′ or 3′) of an oligonucleotide strand, which is formed by one strand extending out of the other strand in a double-stranded oligonucleotide. A single-stranded region extending out of the 3′ terminus and/or 5′ terminus of a duplex is referred to as an overhang.

As used herein, the terms “gene activation”, “activating gene expression”, “gene up-regulation” and “up-regulating gene expression” can be used interchangeably, and mean an increase in transcription, translation, expression or activity of a certain nucleic acid as determined by measuring the transcriptional level, mRNA level, protein level, enzymatic activity, methylation state, chromatin state or configuration, translation level or the activity or state in a cell or biological system of a gene. These activities or states can be determined directly or indirectly. In addition, “gene activation”, “activating gene expression”, “gene up-regulation” or “up-regulating gene expression” refers to an increase in activity associated with a nucleic acid sequence, regardless of the mechanism of such activation. For example, the nucleic acid sequence plays a regulatory role as a regulatory sequence, the nucleic acid sequence is transcribed into RNA and the RNA is translated into a protein, thereby increasing the expression of the protein.

As used herein, the terms “small activating RNA”, “saRNA”, and “small activating nucleic acid molecule” can be used interchangeably, and refer to a nucleic acid molecule that can upregulate target gene expression and can be composed of the first nucleic acid fragment (antisense nucleic acid strand, also referred to as antisense oligonucleotide strand) containing a nucleotide sequence having sequence identity or homology with the non-coding nucleic acid sequence (e.g., a promoter and an enhancer) of a target gene and a second nucleic acid fragment (sense nucleic acid strand, also referred to as sense oligonucleotide strand) containing a nucleotide sequence complementary with the first nucleic acid fragment, wherein the first nucleic acid fragment and the second nucleic acid fragment form a duplex. The small activating nucleic acid molecule can also be composed of a synthesized or vector-expressed single-stranded RNA molecule that can form a hairpin structure by two complementary regions within the molecule, wherein the first region comprises a nucleotide sequence having sequence identity to the target sequence of a promoter of a gene, and the second region comprises a nucleotide sequence which is complementary with the first region. The length of the duplex region of the small activating nucleic acid molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45, or about 50 base pairs. In addition, the terms “saRNA”, “small activating RNA”, and “small activating nucleic acid molecule” also comprise nucleic acids other than the ribonucleotide, including, but not limited to, modified nucleotides or analogues.

As used herein, the term “hot spot” refers to a promoter region of at least 30 bp in length of a target gene, wherein targets of functional small activating nucleic acid molecules are enriched, i.e., at least 30% of the small activating nucleic acid molecules designed to target this region can induce a 1.2-fold or more change in the mRNA expression of the target gene.

As used herein, the term “synthesis” refers to a method for synthesis of an oligonucleotide, including any method allowing RNA synthesis, such as chemical synthesis, in vitro transcription, and/or vector-based expression.

According to the present invention, the expression of LHPP gene is up-regulated by RNA activation, and a related disease (particularly hepatocellular carcinoma) is treated by increasing the expression of full-length LHPP protein. The LHPP gene in the present invention is sometimes also called a target gene.

The method for preparing the small activating nucleic acid molecule provided by the present invention comprises sequence design and synthesis.

Small activating nucleic acid molecules of the present invention can be chemically synthesized or can be obtained from a biotechnology company specialized in nucleic acid synthesis.

Generally speaking, chemical synthesis of nucleic acids comprises the following four steps: (1) synthesis of oligomeric ribonucleotides; (2) deprotection; (3) purification and isolation; and (4) desalination and annealing.

For example, the specific steps for chemically synthesizing saRNAs described herein are as follows:

(1) Synthesis of Oligomeric Ribonucleotides

Synthesis of 1 μM RNA was set in an automatic DNA/RNA synthesizer (e.g., Applied Biosystems EXPEDITE8909), and the coupling time of each cycle was set as 10 to 15 min. With a solid phase-bonded 5′-O-p-dimethoxytriphenylmethyl-thymidine substrate as an initiator, one base was bonded to the solid phase substrate in the first cycle, and then, in the n^{th}(19≥n≥2) cycle, one base was bonded to the base bonded in the n−1^{th} cycle. This process was repeated until the synthesis of the whole nucleic acid sequence was completed.

(2) Deprotection

The solid phase substrate bonded with the saRNA was put into a test tube, and 1 mL of a mixed solution of ethanol and ammonium hydroxide (volume ratio: 1:3) was added to the test tube. The test tube was then sealed and placed in an incubator, and the mixture was incubated at 25-70° C. for 2 to 30 h. The solution containing the solid phase substrate bonded with the saRNA was filtered, and the filtrate was collected. The solid phase substrate was rinsed with double distilled water twice (1 mL each time), and the filtrate was collected. The collected eluent was combined and dried under vacuum for 1 to 12 h. Then the solution was added with 1 mL of a solution of tetrabutylammonium fluoride in tetrahydrofuran (1 M), let stand at room temperature for 4 to 12 h, followed by addition of 2 mL of n-butanol. Precipitate was collected to give a single-stranded crude product of saRNA by high-speed centrifugation.

(3) Purification and Isolation

The resulting crude product of saRNA was dissolved in 2 mL of triethylamine acetate solution with a concentration of 1 mol/L, and the solution was separated by a reversed-phase C18 column of high pressure liquid chromatography to give a purified single-stranded product of saRNA.

(4) Desalination and Annealing

Salts were removed by gel filtration (size exclusion chromatography). A single sense oligomeric ribonucleic acid strand and a single antisense oligomeric ribonucleic acid strand were mixed in a 1 to 2 mL of buffer (10 mM Tris, pH 7.5-8.0, 50 mM NaCl) at a molar ratio of 1:1. The solution was heated to 95° C., and was then slowly cooled to room temperature to give a solution containing saRNA.

It was discovered in this study that after being introduced into a cell, the aforementioned saRNA could effectively increase the mRNA and protein expression of full-length LHPP.

The present invention will be further illustrated with reference to specific examples and drawings below. It should be understood that these examples are merely intended to illustrate the present invention rather than limit the scope of the present invention. In the following examples, study methods without specific conditions were generally in accordance with conventional conditions, such as conditions described in Sambrook, et al., Molecular Cloning: A Laboratory Manual (New York: Cold Spring Harbor Laboratory Press, 1989), or conditions recommended by the manufacturer.

### EXAMPLES

### Example 1

### Design and Synthesis of Small Activating Nucleic Acid Molecule Targeting LHPP Promoter

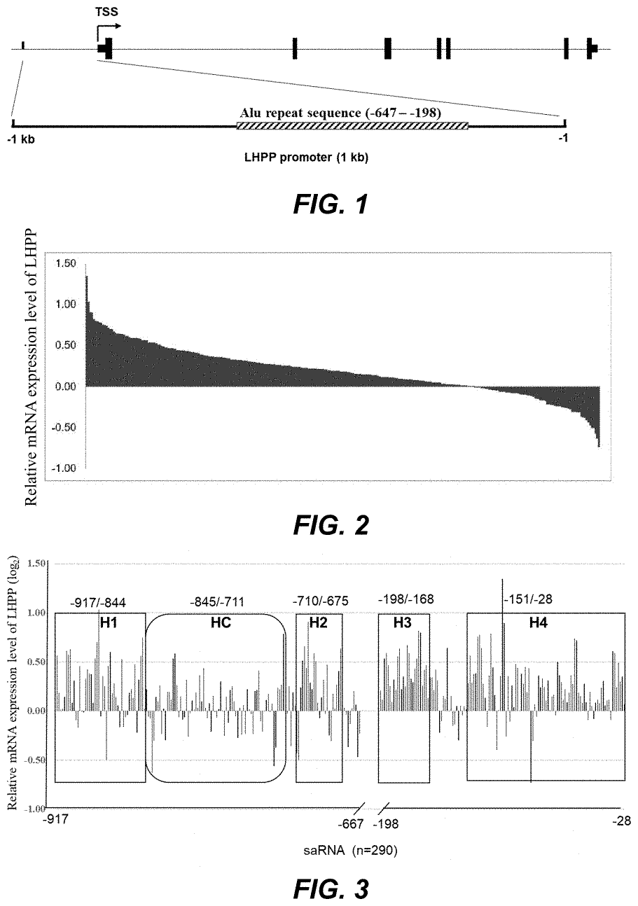

Using a 1 kb sequence (SEQ ID No: 493) encompassing −1 kb to −1 bp, but excluding an Alu repeat sequence from −647 bp to −198 bp, in the LHPP promoter region, as a target sequence (FIG. 1)), a series of 19-nt saRNA target sequences were selected, moving 1 bp each time, and a total of 453 saRNA target sequences were obtained. The target sequences were filtered to keep those which met the following criteria: (1) having GC content between 35% and 65%; (2) with less than 5 consecutive identical nucleotides; (3) with 2 or less dinucleotide repeats; and (4) with 2 or less trinucleotide repeat sequences. After filtration, 290 target sequences remained.

Each of the sense strand and antisense strand in the double-stranded small activating RNA (saRNA) used in the study had 21 nucleotides in length. The 19 nucleotides in the 5′ region of the first nucleic acid strand (sense strand) of the double-stranded saRNA had 100% sequence identity to the target sequence of the promoter, and the 3′ terminus of the first nucleic acid strand contained a TT sequence. The 19 nucleotides in the 5′ region of the second nucleic acid strand were fully complementary with the first ribonucleic acid strand sequence, and the 3′ terminus of the second nucleic acid strand contained a TT sequence. The aforementioned two strands of the double-stranded saRNA were mixed at a molar ratio of 1:1, and after annealed to obtain a duplex saRNA.

The sequence of the LHPP promoter is shown as follows, which corresponds to position 1 to position 1000 from 5′ to 3′ of SEQ ID NO:493:

```
-1000 ttgaacccca taacatttca acgaattcct catcctttct gtgaatcaag
  -950 agcctgaaaa gaaatggtga aataatatga tcctctcttc tttgaaagct
  -900 caaagctatg ttggaccaga agtaaagtgt tctcgtttct atttaataac
  -850 ttgaaaggtt ccgaggggcc attgaggaaa ctcctccctt ttaatatcaa
  -800 tgtgtattta ttgcaaaaat aatgtagcat cgagtggtat tttatagctt
  -750 atccaaaaac ctcctgggtt taacgcattg tgatagtccc gttttcttct
  -700 cagcccaggt cctatgcatc ctcatctatg cagggctgtt atctgcatat
  -650 aatttttttt ttttttaaga caaagtcttg ctctgtcgcc ccggctggag
  -600 tgcagtggtg caatctcggc tcactgcaac ctccgcctcc caggttcaag
  -550 cggttcttcc gcctcagcct accgagtagc tgggactaca ggcatgcgcc
  -500 accacaccta ggtgattttt gtatttttag tagagacagg ggtttcacca
  -450 tgttgaccag gctggtctcg aactcctgat ctcaagcgat ccacccgcct
  -400 cagcctccca aagtgctggg attacaggca taagccacta cgcccggcct
  -350 caattttgta ttgtactttt tctttctttc tttaatagag acagggtctc
  -300 actatgttga ctaggttggt ctagaactcc tgggcacaag ctgtccgccc
  -250 gcttctgcct cccaaagtgc tgggattgca ggcgtgaacc accgcccctg
  -200 gctacaggtg ccttcttgtc tcaatttgcc tttgaccttt cttagggact
  -150 tgttttctgc ttttcctgct ctttgtccgc tgatctcctg ggaagaaagc
  -100 ttccgaaaag gacaccgttt caggggcgag tgacgccggg gtgcccaggc
    -50 cgcgccccag ttccgggttt gcacccggtc ttcttgccct gccccgcccg
```

### Example 2

### High-throughput Screening of saRNAs Targeting LHPP Promoter Region

1. Cell Culture and Transfection

Human liver cancer cell line Huh7 was cultured in DMEM medium (Gibco), containing 10% of calf serum (Sigma-Aldrich) and 1% of penicillin/streptomycin (Gibco). The cells were cultured at 5% CO₂ and 37° C. According to the instructions provided by the manufacturer, RNAiMax (Invitrogen, Carlsbad, Calif.) was used to transfect small activating RNAs at a concentration of 10 nM (unless otherwise specified).

2. One-Step RT-qPCR

At the end of transfection, the media were discarded, and each well was washed with 150 μL of PBS once. After discarding the PBS, 50 μL of cell lysis buffer (Takara) was added to each well and incubated at room temperature for 5 min. 1 μL of the resulted cell lysis was taken from each well and analyzed by qPCR on an ABI 7500 fast real-time PCR system (Applied Biosystems) using a one-step TB Green™ PrimeScrip™ RT-PCR kit II (Takara, RR086A) Each transfection sample was repeatedly amplified in 3 replicate wells. PCR reaction conditions are shown in Table 1 below.

TABLE 1
PCR reaction preparation

| Reagent | Volume/Reaction |
|---|---|
| 2 × One-step TB Green RT-PCR buffer 4 | 2.5 μL |
| PrimeScript 1 step enzyme mixture 2 | 0.2 μL |
| Mixture of forward and reverse primers (5 μM) | 0.4 μL |
| No RNase dH₂O | 1.4 μL |
| Crude lysate (RNA) | 0.5 μL |
| Sum | 5 μL |

Reaction conditions were as follows: reverse transcription reaction (stage 1): 5 min at 42° C., 10 s at 95° C.; PCR reaction (stage 2): 5 s at 95° C., 20 s at 60° C., 45 cycles of amplification. HPRT1 and TBP were used as internal reference genes. PCR primers used for amplifying LHPP, HPRT1 and TBP genes are shown in Table 2, wherein LHPP was amplified using the LHPP F1/R1 primer pair.

TABLE 2
Primer sequences for RT-qPCR analysis

| Primer | Sequence No. | Sequence (5′-3′) |
|---|---|---|
| LHPP F1 | SEQ ID NO: 494 | AAGGCGCTTGAGTATGCCTG |
| LHPP R1 | SEQ ID NO: 495 | GTGGGCTTCCACTCCTATCG |
| HPRT1 F | SEQ ID NO: 496 | ATGGACAGGACTGAACGTCTT |
| HPRT1 R | SEQ ID NO: 497 | TCCAGCAGGTCAGCAAAGAA |
| TBP F | SEQ ID NO: 498 | ATAATCCCAAGCGGTTTGCT |
| TBP R | SEQ ID NO: 499 | CTGCCAGTCTGGACTGTTCT |

To calculate the relative expression level (Eᵣₑₗ) of LHPP (target gene) in an saRNA-transfected sample relative to control treatment (Mock), the Ct values of the target gene and the two internal reference genes were substituted into formula 1

Eᵣₑₗ=2^{(CtT}^{ₘ}^{-CtT}^{ₛ}⁾/((2^{(CtR1}^{ₘ}^{-CtR1}^{ₛ}⁾*2^{(CtR2}^{ₘ}^{-CtR2}^{ₛ}⁾)^{(1/2)})

wherein CtTₘ was the Ct value of the target gene from the control (Mock) sample; CtTₛ was the Ct value of the target gene from the saRNA-treated sample; CtR1m was the Ct value of the internal reference gene 1 from the control (Mock) sample; CtR1s was the Ct value of the internal reference gene 1 from the saRNA-treated sample; CtR2m was the Ct value of the internal reference gene 2 from the control (Mock) sample; and CtR2s was the Ct value of the internal reference gene 2 from the saRNA-treated sample.

3. Screening of Functional saRNAs

In order to obtain saRNAs capable of activating LHPP transcription, Huh7 cells were transfected with each of the aforementioned 290 saRNAs with a transfection concentration of 10 nM, and 72 hours later, according to the same method as described above, the cells were lysed and analyzed by one-step RT-qPCR to obtain the relative (compared with the control (Mock)) expression level of LHPP gene for each saRNA-treated sample. As shown in Table 3, 164 (56.6%) and 37 (12.8%) saRNAs exhibited activating and inhibiting activities, respectively, and 89 (30.7%) saRNAs had no effect on the expression of LHPP. The observed maximum activation and maximum inhibition is 3.46 fold and 0.49 fold, respectively, saRNAs with activating activity are referred to as activating saRNAs, and the saRNAs with inhibiting activity are referred to as inhibiting saRNAs.

TABLE 3
High-throughput screening results of LHPP

| | | Number | |
|---|---|---|---|
| | log₂ value of change in LHPP | of | Per- |
| saRNA activity | (fold) | saRNAs | centage |
| High activation | ≥0.49 (1.50)~≤1.79(3.46) | 30 | 10.3 |
| Moderate activation | ≥0.26 (1.20)~<0.49 (1.50) | 81 | 27.9 |
| Mild activation | ≥0.13 (1.10)~<0.26 (1.20) | 53 | 18.3 |
| No effect | <0.13 (1.10)~>−0.13 (0.91) | 89 | 30.7 |
| Mild inhibition | ≤−0.13 (0.91)~>−0.26 (0.84) | 18 | 6.2 |
| Moderate inhibition | ≤−0.26 (0.84)~>−0.49 (0.71) | 14 | 4.8 |
| High inhibition | ≤−0.49 (0.71)~≥−0.73 (0.49) | 5 | 1.7 |
| Total | | 290 | 100 |

FIG. 2 further shows the distribution of the activities of the LHPP saRNAs sorted from high activation to high inhibition.

TABLE 4
Functional saRNA sequences, functional target sequences thereof
and changes in LHPP mRNA expression level

| | | | | Fold of | Fold of |
|---|---|---|---|---|---|
| | | | | changes | changes in |
| | | | | in | relative |
| | | | | relative | LHPP |
| | | | | LHPP | mRNA |
| | | | | mRNA | expression |
| | Active target | Sense sequence | Antisense sequence | expression | level |
| saRNA | sequence (5′-3′) | (5′-3′) | (5′-3′) | level | (log2) |
| RAG7-133 | GCTCTTTGTCCGCTGATCT | GCUCUUUGUCCGCUGAUCUTT | AGAUCAGCGGACAAAGAGCTT | 2.54 | 1.35 |
| | (SEQ ID NO: 329) | (SEQ ID NO: 1) | (SEQ ID NO: 165) | | |
| RAG7-892 | TGTTGGACCAGAAGTAAAG | UGUUGGACCAGAAGUAAAGTT | CUUUACUUCUGGUCCAACATT | 2.05 | 1.03 |
| | (SEQ ID NO: 330) | (SEQ ID NO: 2) | (SEQ ID NO: 166) | | |
| RAG7-694 | AGGTCCTATGCATCCTCAT | AGGUCCUAUGCAUCCUCAUTT | AUGAGGAUGCAUAGGACCUTT | 1.88 | 0.91 |
| | (SEQ ID NO: 331) | (SEQ ID NO: 3) | (SEQ ID NO: 167) | | |
| RAG7-132 | CTCTTTGTCCGCTGATCTC | CUCUUUGUCCGCUGAUCUCTT | GAGAUCAGCGGACAAAGAGTT | 1.86 | 0.90 |
| | (SEQ ID NO: 332) | (SEQ ID NO: 4) | (SEQ ID NO: 168) | | |
| RAG7-178 | AATTTGCCTTTGACCTTTC | AAUUUGCCUUUGACCUUUCTT | GAAAGGUCAAAGGCAAAUUTT | 1.76 | 0.82 |
| | (SEQ ID NO: 333) | (SEQ ID NO: 5) | (SEQ ID NO: 169) | | |
| RAG7-177 | ATTTGCCTTTGACCTTTCT | AUUUGCCUUUGACCUUUCUTT | AGAAAGGUCAAAGGCAAAUTT | 1.74 | 0.80 |
| | (SEQ ID NO: 334) | (SEQ ID NO: 6) | (SEQ ID NO: 170) | | |
| RAG7-139 | TTTCCTGCTCTTTGTCCGC | UUUCCUGCUCUUUGUCCGCTT | GCGGACAAAGAGCAGGAAATT | 1.73 | 0.79 |
| | (SEQ ID NO: 335) | (SEQ ID NO: 7) | (SEQ ID NO: 171) | | |
| RAG7-707 | TTCTTCTCAGCCCAGGTCC | UUCUUCUCAGCCCAGGUCCTT | GGACCUGGGCUGAGAAGAATT | 1.72 | 0.78 |
| | (SEQ ID NO: 336) | (SEQ ID NO: 8) | (SEQ ID NO: 172) | | |
| RAG7-145 | TCTGCTTTTCCTGCTCTTT | UCUGCUUUUCCUGCUCUUUTT | AAAGAGCAGGAAAAGCAGATT | 1.71 | 0.77 |
| | (SEQ ID NO: 337) | (SEQ ID NO: 9) | (SEQ ID NO: 173) | | |
| RAG7-146 | TTCTGCTTTTCCTGCTCTT | UUCUGCUUUUCCUGCUCUUTT | AAGAGCAGGAAAAGCAGAATT | 1.69 | 0.76 |
| | (SEQ ID NO: 338) | (SEQ ID NO: 10) | (SEQ ID NO: 174) | | |
| RAG7-846 | AAGGTTCCGAGGGGCCATT | AAGGUUCCGAGGGGCCAUUTT | AAUGGCCCCUCGGAACCUUTT | 1.68 | 0.75 |
| | (SEQ ID NO: 339) | (SEQ ID NO: 11) | (SEQ ID NO: 175) | | |
| RAG7-95 | AAAAGGACACCGTTTCAGG | AAAAGGACACCGUUUCAGGTT | CCUGAAACGGUGUCCUUUUTT | 1.67 | 0.74 |
| | (SEQ ID NO: 340) | (SEQ ID NO: 12) | (SEQ ID NO: 176) | | |
| RAG7-94 | AAAGGACACCGTTTCAGGG | AAAGGACACCGUUUCAGGGTT | CCCUGAAACGGUGUCCUUUTT | 1.64 | 0.72 |
| | (SEQ ID NO: 341) | (SEQ ID NO: 13) | (SEQ ID NO: 177) | | |
| RAG7-893 | ATGTTGGACCAGAAGTAAA | AUGUUGGACCAGAAGUAAATT | UUUACUUCUGGUCCAACAUTT | 1.63 | 0.70 |
| | (SEQ ID NO: 342) | (SEQ ID NO: 14) | (SEQ ID NO: 178) | | |
| RAG7-706 | TCTTCTCAGCCCAGGTCCT | UCUUCUCAGCCCAGGUCCUTT | AGGACCUGGGCUGAGAAGATT | 1.63 | 0.70 |
| | (SEQ ID NO: 343) | (SEQ ID NO: 15) | (SEQ ID NO: 179) | | |
| RAG7-184 | TGTCTCAATTTGCCTTTGA | UGUCUCAAUUUGCCUUUGATT | UCAAAGGCAAAUUGAGACATT | 1.59 | 0.67 |
| | (SEQ ID NO: 344) | (SEQ ID NO: 16) | (SEQ ID NO: 180) | | |
| RAG7-696 | CCAGGTCCTATGCATCCTC | CCAGGUCCUAUGCAUCCUCTT | GAGGAUGCAUAGGACCUGGTT | 1.58 | 0.66 |
| | (SEQ ID NO: 345) | (SEQ ID NO: 17) | (SEQ ID NO: 181) | | |
| RAG7-144 | CTGCTTTTCCTGCTCTTTG | CUGCUUUUCCUGCUCUUUGTT | CAAAGAGCAGGAAAAGCAGTT | 1.56 | 0.64 |
| | (SEQ ID NO: 346) | (SEQ ID NO: 18) | (SEQ ID NO: 182) | | |
| RAG7-162 | TTCTTAGGGACTTGTTTTC | UUCUUAGGGACUUGUUUUCTT | GAAAACAAGUCCCUAAGAATT | 1.56 | 0.64 |
| | (SEQ ID NO: 347) | (SEQ ID NO: 19) | (SEQ ID NO: 183) | | |
| RAG7-677 | ATCTATGCAGGGCTGTTAT | AUCUAUGCAGGGCUGUUAUTT | AUAACAGCCCUGCAUAGAUTT | 1.56 | 0.64 |
| | (SEQ ID NO: 348) | (SEQ ID NO: 20) | (SEQ ID NO: 184) | | |
| RAG7-188 | TTCTTGTCTCAATTTGCCT | UUCUUGUCUCAAUUUGCCUTT | AGGCAAAUUGAGACAAGAATT | 1.55 | 0.64 |
| | (SEQ ID NO: 349) | (SEQ ID NO: 21) | (SEQ ID NO: 185) | | |
| RAG7-907 | GAAAGCTCAAAGCTATGTT | GAAAGCUCAAAGCUAUGUUTT | AACAUAGCUUUGAGCUUUCTT | 1.55 | 0.63 |
| | (SEQ ID NO: 350) | (SEQ ID NO: 22) | (SEQ ID NO: 186) | | |
| RAG7-909 | TTGAAAGCTCAAAGCTATG | UUGAAAGCUCAAAGCUAUGTT | CAUAGCUUUGAGCUUUCAATT | 1.53 | 0.61 |
| | (SEQ ID NO: 351) | (SEQ ID NO: 23) | (SEQ ID NO: 187) | | |
| RAG7-35 | GGTTTGCACCCGGTCTTCT | GGUUUGCACCCGGUCUUCUTT | AGAAGACCGGGUGCAAACCTT | 1.53 | 0.61 |
| | (SEQ ID NO: 352) | (SEQ ID NO: 24) | (SEQ ID NO: 188) | | |
| RAG7-886 | ACCAGAAGTAAAGTGTTCT | ACCAGAAGUAAAGUGUUCUTT | AGAACACUUUACUUCUGGUTT | 1.52 | 0.60 |
| | (SEQ ID NO: 353) | (SEQ ID NO: 25) | (SEQ ID NO: 189) | | |
| RAG7-34 | GTTTGCACCCGGTCTTCTT | GUUUGCACCCGGUCUUCUUTT | AAGAAGACCGGGUGCAAACTT | 1.51 | 0.59 |
| | (SEQ ID NO: 354) | (SEQ ID NO: 26) | (SEQ ID NO: 190) | | |
| RAG7-183 | GTCTCAATTTGCCTTTGAC | GUCUCAAUUUGCCUUUGACTT | GUCAAAGGCAAAUUGAGACTT | 1.51 | 0.59 |
| | (SEQ ID NO: 355) | (SEQ ID NO: 27) | (SEQ ID NO: 191) | | |
| RAG7-195 | AGGTGCCTTCTTGTCTCAA | AGGUGCCUUCUUGUCUCAATT | UUGAGACAAGAAGGCACCUTT | 1.51 | 0.59 |
| | (SEQ ID NO: 356) | (SEQ ID NO: 28) | (SEQ ID NO: 192) | | |
| RAG7-829 | TTGAGGAAACTCCTCCCTT | UUGAGGAAACUCCUCCCUUTT | AAGGGAGGAGUUUCCUCAATT | 1.50 | 0.59 |
| | (SEQ ID NO: 357) | (SEQ ID NO: 29) | (SEQ ID NO: 193) | | |
| RAG7-691 | TCCTATGCATCCTCATCTA | UCCUAUGCAUCCUCAUCUATT | UAGAUGAGGAUGCAUAGGATT | 1.50 | 0.58 |
| | (SEQ ID NO: 358) | (SEQ ID NO: 30) | (SEQ ID NO: 194) | | |
| RAG7-908 | TGAAAGCTCAAAGCTATGT | UGAAAGCUCAAAGCUAUGUTT | ACAUAGCUUUGAGCUUUCATT | 1.49 | 0.57 |
| | (SEQ ID NO: 359) | (SEQ ID NO: 31) | (SEQ ID NO: 195) | | |
| RAG7-150 | TGTTTTCTGCTTTTCCTGC | UGUUUUCUGCUUUUCCUGCTT | GCAGGAAAAGCAGAAAACATT | 1.48 | 0.56 |
| | (SEQ ID NO: 360) | (SEQ ID NO: 32) | (SEQ ID NO: 196) | | |
| RAG7-916 | CTCTTCTTTGAAAGCTCAA | CUCUUCUUUGAAAGCUCAATT | UUGAGCUUUCAAAGAAGAGTT | 1.48 | 0.56 |
| | (SEQ ID NO: 361) | (SEQ ID NO: 33) | (SEQ ID NO: 197) | | |
| RAG7-847 | AAAGGTTCCGAGGGGCCAT | AAAGGUUCCGAGGGGCCAUTT | AUGGCCCCUCGGAACCUUUTT | 1.48 | 0.56 |
| | (SEQ ID NO: 362) | (SEQ ID NO: 34) | (SEQ ID NO: 198) | | |
| RAG7-189 | CTTCTTGTCTCAATTTGCC | CUUCUUGUCUCAAUUUGCCTT | GGCAAAUUGAGACAAGAAGTT | 1.47 | 0.56 |
| | (SEQ ID NO: 363) | (SEQ ID NO: 35) | (SEQ ID NO: 199) | | |
| RAG7-830 | ATTGAGGAAACTCCTCCCT | AUUGAGGAAACUCCUCCCUTT | AGGGAGGAGUUUCCUCAAUTT | 1.45 | 0.54 |
| | (SEQ ID NO: 364) | (SEQ ID NO: 36) | (SEQ ID NO: 200) | | |
| RAG7-894 | TATGTTGGACCAGAAGTAA | UAUGUUGGACCAGAAGUAATT | UUACUUCUGGUCCAACAUATT | 1.45 | 0.54 |
| | (SEQ ID NO: 365) | (SEQ ID NO: 37) | (SEQ ID NO: 201) | | |
| RAG7-196 | CAGGTGCCTTCTTGTCTCA | CAGGUGCCUUCUUGUCUCATT | UGAGACAAGAAGGCACCUGTT | 1.45 | 0.54 |
| | (SEQ ID NO: 366) | (SEQ ID NO: 38) | (SEQ ID NO: 202) | | |
| RAG7-179 | CAATTTGCCTTTGACCTTT | CAAUUUGCCUUUGACCUUUTT | AAAGGUCAAAGGCAAAUUGTT | 1.45 | 0.53 |
| | (SEQ ID NO: 367) | (SEQ ID NO: 39) | (SEQ ID NO: 203) | | |
| RAG7-879 | GTAAAGTGTTCTCGTTTCT | GUAAAGUGUUCUCGUUUCUTT | AGAAACGAGAACACUUUACTT | 1.44 | 0.53 |
| | (SEQ ID NO: 368) | (SEQ ID NO: 40) | (SEQ ID NO: 204) | | |
| RAG7-697 | CCCAGGTCCTATGCATCCT | CCCAGGUCCUAUGCAUCCUTT | AGGAUGCAUAGGACCUGGGTT | 1.43 | 0.51 |
| | (SEQ ID NO: 369) | (SEQ ID NO: 41) | (SEQ ID NO: 205) | | |
| RAG7-690 | CCTATGCATCCTCATCTAT | CCUAUGCAUCCUCAUCUAUTT | AUAGAUGAGGAUGCAUAGGTT | 1.42 | 0.51 |
| | (SEQ ID NO: 370) | (SEQ ID NO: 42) | (SEQ ID NO: 206) | | |
| RAG7-104 | AAGCTTCCGAAAAGGACAC | AAGCUUCCGAAAAGGACACTT | GUGUCCUUUUCGGAAGCUUTT | 1.41 | 0.50 |
| | (SEQ ID NO: 371) | (SEQ ID NO: 43) | (SEQ ID NO: 207) | | |
| RAG7-32 | TTGCACCCGGTCTTCTTGC | UUGCACCCGGUCUUCUUGCTT | GCAAGAAGACCGGGUGCAATT | 1.40 | 0.49 |
| | (SEQ ID NO: 372) | (SEQ ID NO: 44) | (SEQ ID NO: 208) | | |
| RAG7-126 | GTCCGCTGATCTCCTGGGA | GUCCGCUGAUCUCCUGGGATT | UCCCAGGAGAUCAGCGGACTT | 1.39 | 0.48 |
| | (SEQ ID NO: 373) | (SEQ ID NO: 45) | (SEQ ID NO: 209) | | |
| RAG7-850 | TTGAAAGGTTCCGAGGGGC | UUGAAAGGUUCCGAGGGGCTT | GCCCCUCGGAACCUUUCAATT | 1.39 | 0.47 |
| | (SEQ ID NO: 374) | (SEQ ID NO: 46) | (SEQ ID NO: 210) | | |
| RAG7-684 | CATCCTCATCTATGCAGGG | CAUCCUCAUCUAUGCAGGGTT | CCCUGCAUAGAUGAGGAUGTT | 1.39 | 0.47 |
| | (SEQ ID NO: 375) | (SEQ ID NO: 47) | (SEQ ID NO: 211) | | |
| RAG7-194 | GGTGCCTTCTTGTCTCAAT | GGUGCCUUCUUGUCUCAAUTT | AUUGAGACAAGAAGGCACCTT | 1.38 | 0.47 |
| | (SEQ ID NO: 376) | (SEQ ID NO: 48) | (SEQ ID NO: 212) | | |
| RAG7-174 | TGCCTTTGACCTTTCTTAG | UGCCUUUGACCUUUCUUAGTT | CUAAGAAAGGUCAAAGGCATT | 1.38 | 0.47 |
| | (SEQ ID NO: 377) | (SEQ ID NO: 49) | (SEQ ID NO: 213) | | |
| RAG7-902 | CTCAAAGCTATGTTGGACC | CUCAAAGCUAUGUUGGACCTT | GGUCCAACAUAGCUUUGAGTT | 1.38 | 0.46 |
| | (SEQ ID NO: 378) | (SEQ ID NO: 50) | (SEQ ID NO: 214) | | |
| RAG7-887 | GACCAGAAGTAAAGTGTTC | GACCAGAAGUAAAGUGUUCTT | GAACACUUUACUUCUGGUCTT | 1.37 | 0.46 |
| | (SEQ ID NO: 379) | (SEQ ID NO: 51) | (SEQ ID NO: 215) | | |
| RAG7-121 | CTGATCTCCTGGGAAGAAA | CUGAUCUCCUGGGAAGAAATT | UUUCUUCCCAGGAGAUCAGTT | 1.36 | 0.45 |
| | (SEQ ID NO: 380) | (SEQ ID NO: 52) | (SEQ ID NO: 216) | | |
| RAG7-138 | TTCCTGCTCTTTGTCCGCT | UUCCUGCUCUUUGUCCGCUTT | AGCGGACAAAGAGCAGGAATT | 1.36 | 0.45 |
| | (SEQ ID NO: 381) | (SEQ ID NO: 53) | (SEQ ID NO: 217) | | |
| RAG7-695 | CAGGTCCTATGCATCCTCA | CAGGUCCUAUGCAUCCUCATT | UGAGGAUGCAUAGGACCUGTT | 1.36 | 0.44 |
| | (SEQ ID NO: 382) | (SEQ ID NO: 54) | (SEQ ID NO: 218) | | |
| RAG7-125 | TCCGCTGATCTCCTGGGAA | UCCGCUGAUCUCCUGGGAATT | UUCCCAGGAGAUCAGCGGATT | 1.36 | 0.44 |
| | (SEQ ID NO: 383) | (SEQ ID NO: 55) | (SEQ ID NO: 219) | | |
| RAG7-776 | TAGCATCGAGTGGTATTTT | UAGCAUCGAGUGGUAUUUUTT | AAAAUACCACUCGAUGCUATT | 1.35 | 0.44 |
| | (SEQ ID NO: 384) | (SEQ ID NO: 56) | (SEQ ID NO: 220) | | |
| RAG7-119 | GATCTCCTGGGAAGAAAGC | GAUCUCCUGGGAAGAAAGCTT | GCUUUCUUCCCAGGAGAUCTT | 1.35 | 0.43 |
| | (SEQ ID NO: 385) | (SEQ ID NO: 57) | (SEQ ID NO: 221) | | |
| RAG7-180 | TCAATTTGCCTTTGACCTT | UCAAUUUGCCUUUGACCUUTT | AAGGUCAAAGGCAAAUUGATT | 1.35 | 0.43 |
| | (SEQ ID NO: 386) | (SEQ ID NO: 58) | (SEQ ID NO: 222) | | |
| RAG7-898 | AAGCTATGTTGGACCAGAA | AAGCUAUGUUGGACCAGAATT | UUCUGGUCCAACAUAGCUUTT | 1.34 | 0.43 |
| | (SEQ ID NO: 387) | (SEQ ID NO: 59) | (SEQ ID NO: 223) | | |
| RAG7-175 | TTGCCTTTGACCTTTCTTA | UUGCCUUUGACCUUUCUUATT | UAAGAAAGGUCAAAGGCAATT | 1.34 | 0.42 |
| | (SEQ ID NO: 388) | (SEQ ID NO: 60) | (SEQ ID NO: 224) | | |
| RAG7-169 | TTGACCTTTCTTAGGGACT | UUGACCUUUCUUAGGGACUTT | AGUCCCUAAGAAAGGUCAATT | 1.34 | 0.42 |
| | (SEQ ID NO: 389) | (SEQ ID NO: 61) | (SEQ ID NO: 225) | | |
| RAG7-720 | TGATAGTCCCGTTTTCTTC | UGAUAGUCCCGUUUUCUUCTT | GAAGAAAACGGGACUAUCATT | 1.33 | 0.41 |
| | (SEQ ID NO: 390) | (SEQ ID NO: 62) | (SEQ ID NO: 226) | | |
| RAG7-678 | CATCTATGCAGGGCTGTTA | CAUCUAUGCAGGGCUGUUATT | UAACAGCCCUGCAUAGAUGTT | 1.33 | 0.41 |
| | (SEQ ID NO: 391) | (SEQ ID NO: 63) | (SEQ ID NO: 227) | | |
| RAG7-917 | TCTCTTCTTTGAAAGCTCA | UCUCUUCUUUGAAAGCUCATT | UGAGCUUUCAAAGAAGAGATT | 1.32 | 0.40 |
| | (SEQ ID NO: 392) | (SEQ ID NO: 64) | (SEQ ID NO: 228) | | |
| RAG7-897 | AGCTATGTTGGACCAGAAG | AGCUAUGUUGGACCAGAAGTT | CUUCUGGUCCAACAUAGCUTT | 1.31 | 0.39 |
| | (SEQ ID NO: 393) | (SEQ ID NO: 65) | (SEQ ID NO: 229) | | |
| RAG7-147 | TTTCTGCTTTTCCTGCTCT | UUUCUGCUUUUCCUGCUCUTT | AGAGCAGGAAAAGCAGAAATT | 1.31 | 0.39 |
| | (SEQ ID NO: 394) | (SEQ ID NO: 66) | (SEQ ID NO: 230) | | |
| RAG7-148 | TTTTCTGCTTTTCCTGCTC | UUUUCUGCUUUUCCUGCUCTT | GAGCAGGAAAAGCAGAAAATT | 1.30 | 0.38 |
| | (SEQ ID NO: 395) | (SEQ ID NO: 67) | (SEQ ID NO: 231) | | |
| RAG7-123 | CGCTGATCTCCTGGGAAGA | CGCUGAUCUCCUGGGAAGATT | UCUUCCCAGGAGAUCAGCGTT | 1.30 | 0.38 |
| | (SEQ ID NO: 396) | (SEQ ID NO: 68) | (SEQ ID NO: 232) | | |
| RAG7-896 | GCTATGTTGGACCAGAAGT | GCUAUGUUGGACCAGAAGUTT | ACUUCUGGUCCAACAUAGCTT | 1.30 | 0.38 |
| | (SEQ ID NO: 397) | (SEQ ID NO: 69) | (SEQ ID NO: 233) | | |
| RAG7-778 | TGTAGCATCGAGTGGTATT | UGUAGCAUCGAGUGGUAUUTT | AAUACCACUCGAUGCUACATT | 1.29 | 0.37 |
| | (SEQ ID NO: 398) | (SEQ ID NO: 70) | (SEQ ID NO: 234) | | |
| RAG7-97 | CGAAAAGGACACCGTTTCA | CGAAAAGGACACCGUUUCATT | UGAAACGGUGUCCUUUUCGTT | 1.29 | 0.37 |
| | (SEQ ID NO: 399) | (SEQ ID NO: 71) | (SEQ ID NO: 235) | | |
| RAG7-103 | AGCTTCCGAAAAGGACACC | AGCUUCCGAAAAGGACACCTT | GGUGUCCUUUUCGGAAGCUTT | 1.29 | 0.37 |
| | (SEQ ID NO: 400) | (SEQ ID NO: 72) | (SEQ ID NO: 236) | | |
| RAG7-114 | CCTGGGAAGAAAGCTTCCG | CCUGGGAAGAAAGCUUCCGTT | CGGAAGCUUUCUUCCCAGGTT | 1.28 | 0.36 |
| | (SEQ ID NO: 401) | (SEQ ID NO: 73) | (SEQ ID NO: 237) | | |
| RAG7-140 | TTTTCCTGCTCTTTGTCCG | UUUUCCUGCUCUUUGUCCGTT | CGGACAAAGAGCAGGAAAATT | 1.28 | 0.36 |
| | (SEQ ID NO: 402) | (SEQ ID NO: 74) | (SEQ ID NO: 238) | | |
| RAG7-134 | TGCTCTTTGTCCGCTGATC | UGCUCUUUGUCCGCUGAUCTT | GAUCAGCGGACAAAGAGCATT | 1.28 | 0.36 |
| | (SEQ ID NO: 403) | (SEQ ID NO: 75) | (SEQ ID NO: 239) | | |
| RAG7-890 | TTGGACCAGAAGTAAAGTG | UUGGACCAGAAGUAAAGUGTT | CACUUUACUUCUGGUCCAATT | 1.28 | 0.36 |
| | (SEQ ID NO: 404) | (SEQ ID NO: 76) | (SEQ ID NO: 240) | | |
| RAG7-130 | CTTTGTCCGCTGATCTCCT | CUUUGUCCGCUGAUCUCCUTT | AGGAGAUCAGCGGACAAAGTT | 1.28 | 0.36 |
| | (SEQ ID NO: 405) | (SEQ ID NO: 77) | (SEQ ID NO: 241) | | |
| RAG7-186 | CTTGTCTCAATTTGCCTTT | CUUGUCUCAAUUUGCCUUUTT | AAAGGCAAAUUGAGACAAGTT | 1.28 | 0.35 |
| | (SEQ ID NO: 406) | (SEQ ID NO: 78) | (SEQ ID NO: 242) | | |
| RAG7-29 | CACCCGGTCTTCTTGCCCT | CACCCGGUCUUCUUGCCCUTT | AGGGCAAGAAGACCGGGUGTT | 1.28 | 0.35 |
| | (SEQ ID NO: 407) | (SEQ ID NO: 79) | (SEQ ID NO: 243) | | |
| RAG7-171 | CTTTGACCTTTCTTAGGGA | CUUUGACCUUUCUUAGGGATT | UCCCUAAGAAAGGUCAAAGTT | 1.27 | 0.34 |
| | (SEQ ID NO: 408) | (SEQ ID NO: 80) | (SEQ ID NO: 244) | | |
| RAG7-172 | CCTTTGACCTTTCTTAGGG | CCUUUGACCUUUCUUAGGGTT | CCCUAAGAAAGGUCAAAGGTT | 1.27 | 0.34 |
| | (SEQ ID NO: 409) | (SEQ ID NO: 81) | (SEQ ID NO: 245) | | |
| RAG7-112 | TGGGAAGAAAGCTTCCGAA | UGGGAAGAAAGCUUCCGAATT | UUCGGAAGCUUUCUUCCCATT | 1.26 | 0.33 |
| | (SEQ ID NO: 410) | (SEQ ID NO: 82) | (SEQ ID NO: 246) | | |
| RAG7-676 | TCTATGCAGGGCTGTTATC | UCUAUGCAGGGCUGUUAUCTT | GAUAACAGCCCUGCAUAGATT | 1.26 | 0.33 |
| | (SEQ ID NO: 411) | (SEQ ID NO: 83) | (SEQ ID NO: 247) | | |
| RAG7-899 | AAAGCTATGTTGGACCAGA | AAAGCUAUGUUGGACCAGATT | UCUGGUCCAACAUAGCUUUTT | 1.26 | 0.33 |
| | (SEQ ID NO: 412) | (SEQ ID NO: 84) | (SEQ ID NO: 248) | | |
| RAG7-182 | TCTCAATTTGCCTTTGACC | UCUCAAUUUGCCUUUGACCTT | GGUCAAAGGCAAAUUGAGATT | 1.26 | 0.33 |
| | (SEQ ID NO: 413) | (SEQ ID NO: 85) | (SEQ ID NO: 249) | | |
| RAG7-686 | TGCATCCTCATCTATGCAG | UGCAUCCUCAUCUAUGCAGTT | CUGCAUAGAUGAGGAUGCATT | 1.25 | 0.32 |
| | (SEQ ID NO: 414) | (SEQ ID NO: 86) | (SEQ ID NO: 250) | | |
| RAG7-848 | GAAAGGTTCCGAGGGGCCA | GAAAGGUUCCGAGGGGCCATT | UGGCCCCUCGGAACCUUUCTT | 1.25 | 0.32 |
| | (SEQ ID NO: 415) | (SEQ ID NO: 87) | (SEQ ID NO: 251) | | |
| RAG7-191 | GCCTTCTTGTCTCAATTTG | GCCUUCUUGUCUCAAUUUGTT | CAAAUUGAGACAAGAAGGCTT | 1.25 | 0.32 |
| | (SEQ ID NO: 416) | (SEQ ID NO: 88) | (SEQ ID NO: 252) | | |
| RAG7-821 | ACTCCTCCCTTTTAATATC | ACUCCUCCCUUUUAAUAUCTT | GAUAUUAAAAGGGAGGAGUTT | 1.25 | 0.32 |
| | (SEQ ID NO: 417) | (SEQ ID NO: 89) | (SEQ ID NO: 253) | | |
| RAG7-109 | GAAGAAAGCTTCCGAAAAG | GAAGAAAGCUUCCGAAAAGTT | CUUUUCGGAAGCUUUCUUCTT | 1.24 | 0.31 |
| | (SEQ ID NO: 418) | (SEQ ID NO: 90) | (SEQ ID NO: 254) | | |
| RAG7-168 | TGACCTTTCTTAGGGACTT | UGACCUUUCUUAGGGACUUTT | AAGUCCCUAAGAAAGGUCATT | 1.24 | 0.31 |
| | (SEQ ID NO: 419) | (SEQ ID NO: 91) | (SEQ ID NO: 255) | | |
| RAG7-905 | AAGCTCAAAGCTATGTTGG | AAGCUCAAAGCUAUGUUGGTT | CCAACAUAGCUUUGAGCUUTT | 1.24 | 0.31 |
| | (SEQ ID NO: 420) | (SEQ ID NO: 92) | (SEQ ID NO: 256) | | |
| RAG7-43 | CAGTTCCGGGTTTGCACCC | CAGUUCCGGGUUUGCACCCTT | GGGUGCAAACCCGGAACUGTT | 1.23 | 0.30 |
| | (SEQ ID NO: 421) | (SEQ ID NO: 93) | (SEQ ID NO: 257) | | |
| RAG7-31 | TGCACCCGGTCTTCTTGCC | UGCACCCGGUCUUCUUGCCTT | GGCAAGAAGACCGGGUGCATT | 1.23 | 0.30 |
| | (SEQ ID NO: 422) | (SEQ ID NO: 94) | (SEQ ID NO: 258) | | |
| RAG7-741 | CCTCCTGGGTTTAACGCAT | CCUCCUGGGUUUAACGCAUTT | AUGCGUUAAACCCAGGAGGTT | 1.23 | 0.30 |
| | (SEQ ID NO: 423) | (SEQ ID NO: 95) | (SEQ ID NO: 259) | | |
| RAG7-102 | GCTTCCGAAAAGGACACCG | GCUUCCGAAAAGGACACCGTT | CGGUGUCCUUUUCGGAAGCTT | 1.23 | 0.30 |
| | (SEQ ID NO: 424) | (SEQ ID NO: 96) | (SEQ ID NO: 260) | | |
| RAG7-181 | CTCAATTTGCCTTTGACCT | CUCAAUUUGCCUUUGACCUTT | AGGUCAAAGGCAAAUUGAGTT | 1.22 | 0.29 |
| | (SEQ ID NO: 425) | (SEQ ID NO: 97) | (SEQ ID NO: 261) | | |
| RAG7-693 | GGTCCTATGCATCCTCATC | GGUCCUAUGCAUCCUCAUCTT | GAUGAGGAUGCAUAGGACCTT | 1.22 | 0.29 |
| | (SEQ ID NO: 426) | (SEQ ID NO: 98) | (SEQ ID NO: 262) | | |
| RAG7-149 | GTTTTCTGCTTTTCCTGCT | GUUUUCUGCUUUUCCUGCUTT | AGCAGGAAAAGCAGAAAACTT | 1.22 | 0.29 |
| | (SEQ ID NO: 427) | (SEQ ID NO: 99) | (SEQ ID NO: 263) | | |
| RAG7-151 | TTGTTTTCTGCTTTTCCTG | UUGUUUUCUGCUUUUCCUGTT | CAGGAAAAGCAGAAAACAATT | 1.22 | 0.28 |
| | (SEQ ID NO: 428) | (SEQ ID NO: 100) | (SEQ ID NO: 264) | | |
| RAG7-884 | CAGAAGTAAAGTGTTCTCG | CAGAAGUAAAGUGUUCUCGTT | CGAGAACACUUUACUUCUGTT | 1.22 | 0.28 |
| | (SEQ ID NO: 429) | (SEQ ID NO: 101) | (SEQ ID NO: 265) | | |
| RAG7-143 | TGCTTTTCCTGCTCTTTGT | UGCUUUUCCUGCUCUUUGUTT | ACAAAGAGCAGGAAAAGCATT | 1.21 | 0.28 |
| | (SEQ ID NO: 430) | (SEQ ID NO: 102) | (SEQ ID NO: 266) | | |
| RAG7-122 | GCTGATCTCCTGGGAAGAA | GCUGAUCUCCUGGGAAGAATT | UUCUUCCCAGGAGAUCAGCTT | 1.21 | 0.28 |
| | (SEQ ID NO: 431) | (SEQ ID NO: 103) | (SEQ ID NO: 267) | | |
| RAG7-89 | ACACCGTTTCAGGGGCGAG | ACACCGUUUCAGGGGCGAGTT | CUCGCCCCUGAAACGGUGUTT | 1.21 | 0.28 |
| | (SEQ ID NO: 432) | (SEQ ID NO: 104) | (SEQ ID NO: 268) | | |
| RAG7-96 | GAAAAGGACACCGTTTCAG | GAAAAGGACACCGUUUCAGTT | CUGAAACGGUGUCCUUUUCTT | 1.21 | 0.27 |
| | (SEQ ID NO: 433) | (SEQ ID NO: 105) | (SEQ ID NO: 269) | | |
| RAG7-708 | TTTCTTCTCAGCCCAGGTC | UUUCUUCUCAGCCCAGGUCTT | GACCUGGGCUGAGAAGAAATT | 1.21 | 0.27 |
| | (SEQ ID NO: 434) | (SEQ ID NO: 106) | (SEQ ID NO: 270) | | |
| RAG7-679 | TCATCTATGCAGGGCTGTT | UCAUCUAUGCAGGGCUGUUTT | AACAGCCCUGCAUAGAUGATT | 1.20 | 0.27 |
| | (SEQ ID NO: 435) | (SEQ ID NO: 107) | (SEQ ID NO: 271) | | |
| RAG7-828 | TGAGGAAACTCCTCCCTTT | UGAGGAAACUCCUCCCUUUTT | AAAGGGAGGAGUUUCCUCATT | 1.20 | 0.26 |
| | (SEQ ID NO: 436) | (SEQ ID NO: 108) | (SEQ ID NO: 272) | | |
| RAG7-837 | AGGGGCCATTGAGGAAACT | AGGGGCCAUUGAGGAAACUTT | AGUUUCCUCAAUGGCCCCUTT | 1.20 | 0.26 |
| | (SEQ ID NO: 437) | (SEQ ID NO: 109) | (SEQ ID NO: 273) | | |
| RAG7-176 | TTTGCCTTTGACCTTTCTT | UUUGCCUUUGACCUUUCUUTT | AAGAAAGGUCAAAGGCAAATT | 1.20 | 0.26 |
| | (SEQ ID NO: 438) | (SEQ ID NO: 110) | (SEQ ID NO: 274) | | |
| RAG7-128 | TTGTCCGCTGATCTCCTGG | UUGUCCGCUGAUCUCCUGGTT | CCAGGAGAUCAGCGGACAATT | 1.20 | 0.26 |
| | (SEQ ID NO: 439) | (SEQ ID NO: 111) | (SEQ ID NO: 275) | | |
| RAG7-704 | TTCTCAGCCCAGGTCCTAT | UUCUCAGCCCAGGUCCUAUTT | AUAGGACCUGGGCUGAGAATT | 1.19 | 0.26 |
| | (SEQ ID NO: 440) | (SEQ ID NO: 112) | (SEQ ID NO: 276) | | |
| RAG7-193 | GTGCCTTCTTGTCTCAATT | GUGCCUUCUUGUCUCAAUUTT | AAUUGAGACAAGAAGGCACTT | 1.19 | 0.26 |
| | (SEQ ID NO: 441) | (SEQ ID NO: 113) | (SEQ ID NO: 277) | | |
| RAG7-735 | GGGTTTAACGCATTGTGAT | GGGUUUAACGCAUUGUGAUTT | AUCACAAUGCGUUAAACCCTT | 1.19 | 0.25 |
| | (SEQ ID NO: 442) | (SEQ ID NO: 114) | (SEQ ID NO: 278) | | |
| RAG7-889 | TGGACCAGAAGTAAAGTGT | UGGACCAGAAGUAAAGUGUTT | ACACUUUACUUCUGGUCCATT | 1.19 | 0.25 |
| | (SEQ ID NO: 443) | (SEQ ID NO: 115) | (SEQ ID NO: 279) | | |
| RAG7-185 | TTGTCTCAATTTGCCTTTG | UUGUCUCAAUUUGCCUUUGTT | CAAAGGCAAAUUGAGACAATT | 1.19 | 0.25 |
| | (SEQ ID NO: 444) | (SEQ ID NO: 116) | (SEQ ID NO: 280) | | |
| RAG7-111 | GGGAAGAAAGCTTCCGAAA | GGGAAGAAAGCUUCCGAAATT | UUUCGGAAGCUUUCUUCCCTT | 1.18 | 0.24 |
| | (SEQ ID NO: 445) | (SEQ ID NO: 117) | (SEQ ID NO: 281) | | |
| RAG7-698 | GCCCAGGTCCTATGCATCC | GCCCAGGUCCUAUGCAUCCTT | GGAUGCAUAGGACCUGGGCTT | 1.18 | 0.24 |
| | (SEQ ID NO: 446) | (SEQ ID NO: 118) | (SEQ ID NO: 282) | | |
| RAG7-33 | TTTGCACCCGGTCTTCTTG | UUUGCACCCGGUCUUCUUGTT | CAAGAAGACCGGGUGCAAATT | 1.18 | 0.24 |
| | (SEQ ID NO: 447) | (SEQ ID NO: 119) | (SEQ ID NO: 283) | | |
| RAG7-113 | CTGGGAAGAAAGCTTCCGA | CUGGGAAGAAAGCUUCCGATT | UCGGAAGCUUUCUUCCCAGTT | 1.18 | 0.24 |
| | (SEQ ID NO: 448) | (SEQ ID NO: 120) | (SEQ ID NO: 284) | | |
| RAG7-44 | CCAGTTCCGGGTTTGCACC | CCAGUUCCGGGUUUGCACCTT | GGUGCAAACCCGGAACUGGTT | 1.18 | 0.24 |
| | (SEQ ID NO: 449) | (SEQ ID NO: 121) | (SEQ ID NO: 285) | | |
| RAG7-710 | GTTTTCTTCTCAGCCCAGG | GUUUUCUUCUCAGCCCAGGTT | CCUGGGCUGAGAAGAAAACTT | 1.18 | 0.24 |
| | (SEQ ID NO: 450) | (SEQ ID NO: 122) | (SEQ ID NO: 286) | | |
| RAG7-187 | TCTTGTCTCAATTTGCCTT | UCUUGUCUCAAUUUGCCUUTT | AAGGCAAAUUGAGACAAGATT | 1.17 | 0.22 |
| | (SEQ ID NO: 451) | (SEQ ID NO: 123) | (SEQ ID NO: 287) | | |
| RAG7-692 | GTCCTATGCATCCTCATCT | GUCCUAUGCAUCCUCAUCUTT | AGAUGAGGAUGCAUAGGACTT | 1.17 | 0.22 |
| | (SEQ ID NO: 452) | (SEQ ID NO: 124) | (SEQ ID NO: 288) | | |
| RAG7-100 | TTCCGAAAAGGACACCGTT | UUCCGAAAAGGACACCGUUTT | AACGGUGUCCUUUUCGGAATT | 1.17 | 0.22 |
| | (SEQ ID NO: 453) | (SEQ ID NO: 125) | (SEQ ID NO: 289) | | |
| RAG7-709 | TTTTCTTCTCAGCCCAGGT | UUUUCUUCUCAGCCCAGGUTT | ACCUGGGCUGAGAAGAAAATT | 1.17 | 0.22 |
| | (SEQ ID NO: 454) | (SEQ ID NO: 126) | (SEQ ID NO: 290) | | |
| RAG7-726 | GCATTGTGATAGTCCCGTT | GCAUUGUGAUAGUCCCGUUTT | AACGGGACUAUCACAAUGCTT | 1.17 | 0.22 |
| | (SEQ ID NO: 455) | (SEQ ID NO: 127) | (SEQ ID NO: 291) | | |
| RAG7-852 | ACTTGAAAGGTTCCGAGGG | ACUUGAAAGGUUCCGAGGGTT | CCCUCGGAACCUUUCAAGUTT | 1.17 | 0.22 |
| | (SEQ ID NO: 456) | (SEQ ID NO: 128) | (SEQ ID NO: 292) | | |
| RAG7-844 | GGTTCCGAGGGGCCATTGA | GGUUCCGAGGGGCCAUUGATT | UCAAUGGCCCCUCGGAACCTT | 1.17 | 0.22 |
| | (SEQ ID NO: 457) | (SEQ ID NO: 129) | (SEQ ID NO: 293) | | |
| RAG7-190 | CCTTCTTGTCTCAATTTGC | CCUUCUUGUCUCAAUUUGCTT | GCAAAUUGAGACAAGAAGGTT | 1.16 | 0.22 |
| | (SEQ ID NO: 458) | (SEQ ID NO: 130) | (SEQ ID NO: 294) | | |
| RAG7-736 | TGGGTTTAACGCATTGTGA | UGGGUUUAACGCAUUGUGATT | UCACAAUGCGUUAAACCCATT | 1.16 | 0.22 |
| | (SEQ ID NO: 459) | (SEQ ID NO: 131) | (SEQ ID NO: 295) | | |
| RAG7-170 | TTTGACCTTTCTTAGGGAC | UUUGACCUUUCUUAGGGACTT | GUCCCUAAGAAAGGUCAAATT | 1.16 | 0.21 |
| | (SEQ ID NO: 460) | (SEQ ID NO: 132) | (SEQ ID NO: 296) | | |
| RAG7-721 | GTGATAGTCCCGTTTTCTT | GUGAUAGUCCCGUUUUCUUTT | AAGAAAACGGGACUAUCACTT | 1.16 | 0.21 |
| | (SEQ ID NO: 461) | (SEQ ID NO: 133) | (SEQ ID NO: 297) | | |
| RAG7-198 | TACAGGTGCCTTCTTGTCT | UACAGGUGCCUUCUUGUCUTT | AGACAAGAAGGCACCUGUATT | 1.16 | 0.21 |
| | (SEQ ID NO: 462) | (SEQ ID NO: 134) | (SEQ ID NO: 298) | | |
| RAG7-722 | TGTGATAGTCCCGTTTTCT | UGUGAUAGUCCCGUUUUCUTT | AGAAAACGGGACUAUCACATT | 1.15 | 0.21 |
| | (SEQ ID NO: 463) | (SEQ ID NO: 135) | (SEQ ID NO: 299) | | |
| RAG7-670 | CAGGGCTGTTATCTGCATA | CAGGGCUGUUAUCUGCAUATT | UAUGCAGAUAACAGCCCUGTT | 1.15 | 0.20 |
| | (SEQ ID NO: 464) | (SEQ ID NO: 136) | (SEQ ID NO: 300) | | |
| RAG7-86 | CCGTTTCAGGGGCGAGTGA | CCGUUUCAGGGGCGAGUGATT | UCACUCGCCCCUGAAACGGTT | 1.15 | 0.20 |
| | (SEQ ID NO: 465) | (SEQ ID NO: 137) | (SEQ ID NO: 301) | | |
| RAG7-833 | GCCATTGAGGAAACTCCTC | GCCAUUGAGGAAACUCCUCTT | GAGGAGUUUCCUCAAUGGCTT | 1.14 | 0.20 |
| | (SEQ ID NO: 466) | (SEQ ID NO: 138) | (SEQ ID NO: 302) | | |
| RAG7-832 | CCATTGAGGAAACTCCTCC | CCAUUGAGGAAACUCCUCCTT | GGAGGAGUUUCCUCAAUGGTT | 1.14 | 0.19 |
| | (SEQ ID NO: 467) | (SEQ ID NO: 139) | (SEQ ID NO: 303) | | |
| RAG7-702 | CTCAGCCCAGGTCCTATGC | CUCAGCCCAGGUCCUAUGCTT | GCAUAGGACCUGGGCUGAGTT | 1.14 | 0.19 |
| | (SEQ ID NO: 468) | (SEQ ID NO: 140) | (SEQ ID NO: 304) | | |
| RAG7-120 | TGATCTCCTGGGAAGAAAG | UGAUCUCCUGGGAAGAAAGTT | CUUUCUUCCCAGGAGAUCATT | 1.14 | 0.19 |
| | (SEQ ID NO: 469) | (SEQ ID NO: 141) | (SEQ ID NO: 305) | | |
| RAG7-780 | AATGTAGCATCGAGTGGTA | AAUGUAGCAUCGAGUGGUATT | UACCACUCGAUGCUACAUUTT | 1.14 | 0.19 |
| | (SEQ ID NO: 470) | (SEQ ID NO: 142) | (SEQ ID NO: 306) | | |
| RAG7-914 | CTTCTTTGAAAGCTCAAAG | CUUCUUUGAAAGCUCAAAGTT | CUUUGAGCUUUCAAAGAAGTT | 1.14 | 0.19 |
| | (SEQ ID NO: 471) | (SEQ ID NO: 143) | (SEQ ID NO: 307) | | |
| RAG7-93 | AAGGACACCGTTTCAGGGG | AAGGACACCGUUUCAGGGGTT | CCCCUGAAACGGUGUCCUUTT | 1.14 | 0.19 |
| | (SEQ ID NO: 472) | (SEQ ID NO: 144) | (SEQ ID NO: 308) | | |
| RAG7-98 | CCGAAAAGGACACCGTTTC | CCGAAAAGGACACCGUUUCTT | GAAACGGUGUCCUUUUCGGTT | 1.14 | 0.19 |
| | (SEQ ID NO: 473) | (SEQ ID NO: 145) | (SEQ ID NO: 309) | | |
| RAG7-853 | AACTTGAAAGGTTCCGAGG | AACUUGAAAGGUUCCGAGGTT | CCUCGGAACCUUUCAAGUUTT | 1.14 | 0.18 |
| | (SEQ ID NO: 474) | (SEQ ID NO: 146) | (SEQ ID NO: 310) | | |
| RAG7-885 | CCAGAAGTAAAGTGTTCTC | CCAGAAGUAAAGUGUUCUCTT | GAGAACACUUUACUUCUGGTT | 1.14 | 0.18 |
| | (SEQ ID NO: 475) | (SEQ ID NO: 147) | (SEQ ID NO: 311) | | |
| RAG7-715 | GTCCCGTTTTCTTCTCAGC | GUCCCGUUUUCUUCUCAGCTT | GCUGAGAAGAAAACGGGACTT | 1.13 | 0.18 |
| | (SEQ ID NO: 476) | (SEQ ID NO: 148) | (SEQ ID NO: 312) | | |
| RAG7-681 | CCTCATCTATGCAGGGCTG | CCUCAUCUAUGCAGGGCUGTT | CAGCCCUGCAUAGAUGAGGTT | 1.13 | 0.17 |
| | (SEQ ID NO: 477) | (SEQ ID NO: 149) | (SEQ ID NO: 313) | | |
| RAG7-106 | GAAAGCTTCCGAAAAGGAC | GAAAGCUUCCGAAAAGGACTT | GUCCUUUUCGGAAGCUUUCTT | 1.12 | 0.17 |
| | (SEQ ID NO: 478) | (SEQ ID NO: 150) | (SEQ ID NO: 314) | | |
| RAG7-137 | TCCTGCTCTTTGTCCGCTG | UCCUGCUCUUUGUCCGCUGTT | CAGCGGACAAAGAGCAGGATT | 1.12 | 0.16 |
| | (SEQ ID NO: 479) | (SEQ ID NO: 151) | (SEQ ID NO: 315) | | |
| RAG7-165 | CCTTTCTTAGGGACTTGTT | CCUUUCUUAGGGACUUGUUTT | AACAAGUCCCUAAGAAAGGTT | 1.11 | 0.16 |
| | (SEQ ID NO: 480) | (SEQ ID NO: 152) | (SEQ ID NO: 316) | | |
| RAG7-160 | CTTAGGGACTTGTTTTCTG | CUUAGGGACUUGUUUUCUGTT | CAGAAAACAAGUCCCUAAGTT | 1.11 | 0.15 |
| | (SEQ ID NO: 481) | (SEQ ID NO: 153) | (SEQ ID NO: 317) | | |
| RAG7-745 | AAAACCTCCTGGGTTTAAC | AAAACCUCCUGGGUUUAACTT | GUUAAACCCAGGAGGUUUUTT | 1.11 | 0.15 |
| | (SEQ ID NO: 482) | (SEQ ID NO: 154) | (SEQ ID NO: 318) | | |
| RAG7-92 | AGGACACCGTTTCAGGGGC | AGGACACCGUUUCAGGGGCTT | GCCCCUGAAACGGUGUCCUTT | 1.11 | 0.15 |
| | (SEQ ID NO: 483) | (SEQ ID NO: 155) | (SEQ ID NO: 319) | | |
| RAG7-107 | AGAAAGCTTCCGAAAAGGA | AGAAAGCUUCCGAAAAGGATT | UCCUUUUCGGAAGCUUUCUTT | 1.11 | 0.15 |
| | (SEQ ID NO: 484) | (SEQ ID NO: 156) | (SEQ ID NO: 320) | | |
| RAG7-826 | AGGAAACTCCTCCCTTTTA | AGGAAACUCCUCCCUUUUATT | UAAAAGGGAGGAGUUUCCUTT | 1.11 | 0.15 |
| | (SEQ ID NO: 485) | (SEQ ID NO: 157) | (SEQ ID NO: 321) | | |
| RAG7-839 | CGAGGGGCCATTGAGGAAA | CGAGGGGCCAUUGAGGAAATT | UUUCCUCAAUGGCCCCUCGTT | 1.11 | 0.15 |
| | (SEQ ID NO: 486) | (SEQ ID NO: 158) | (SEQ ID NO: 322) | | |
| RAG7-738 | CCTGGGTTTAACGCATTGT | CCUGGGUUUAACGCAUUGUTT | ACAAUGCGUUAAACCCAGGTT | 1.11 | 0.15 |
| | (SEQ ID NO: 487) | (SEQ ID NO: 159) | (SEQ ID NO: 323) | | |
| RAG7-911 | CTTTGAAAGCTCAAAGCTA | CUUUGAAAGCUCAAAGCUATT | UAGCUUUGAGCUUUCAAAGTT | 1.11 | 0.15 |
| | (SEQ ID NO: 488) | (SEQ ID NO: 160) | (SEQ ID NO: 324) | | |
| RAG7-883 | AGAAGTAAAGTGTTCTCGT | AGAAGUAAAGUGUUCUCGUTT | ACGAGAACACUUUACUUCUTT | 1.11 | 0.14 |
| | (SEQ ID NO: 489) | (SEQ ID NO: 161) | (SEQ ID NO: 325) | | |
| RAG7-687 | ATGCATCCTCATCTATGCA | AUGCAUCCUCAUCUAUGCATT | UGCAUAGAUGAGGAUGCAUTT | 1.10 | 0.14 |
| | (SEQ ID NO: 490) | (SEQ ID NO: 162) | (SEQ ID NO: 326) | | |
| RAG7-851 | CTTGAAAGGTTCCGAGGGG | CUUGAAAGGUUCCGAGGGGTT | CCCCUCGGAACCUUUCAAGTT | 1.10 | 0.14 |
| | (SEQ ID NO: 491) | (SEQ ID NO: 163) | (SEQ ID NO: 327) | | |
| RAG7-142 | GCTTTTCCTGCTCTTTGTC | GCUUUUCCUGCUCUUUGUCTT | GACAAAGAGCAGGAAAAGCTT | 1.10 | 0.14 |
| | (SEQ ID NO: 492) | (SEQ ID NO: 164) | (SEQ ID NO: 328) | | |

When the 290 saRNAs were sorted by their targeting positions on the LHPP promoter, it can be clearly seen that the functional saRNAs were distributed across the promoter region in a cluster fashion, i.e., at certain promoter regions, there were “hot spots” where functional sRNAs were enriched (FIG. 3). As shown in FIG. 3, there are five hot spots highly enriched for activating saRNAs in the region (H1) from −917 to −844, the region (H2) from −710 to −675, the region (H3) from −198 to −168, the region (H4) from −151 to −28, and the region (H5) from −845 to −711 of the promoter. This study result indicates that the activating saRNAs are not randomly distributed on the promoter, but instead they are enriched in the specific hot spot regions.

The sequence of the hot spot H1 (5′ to 3′: −917 to −844) corresponds to position 1 to position 74 from 5′ to 3′ of SEQ ID NO: 500:

tctcttc tttgaaagct caaagctatg ttggaccaga
agtaaagtgt tctcgtttct atttaataac ttgaaag

The sequence of the hot spot H2 (5′ to 3′: −710 to −675) corresponds to position 1 to position 36 from 5′ to 3′ of SEQ ID NO: 501:

gttttcttct cagcccaggt cctatgcatc ctcatc

The sequence of the hot spot H3 (5′ to 3′: −198 to −168) corresponds to position 1 to position 31 from 5′ to 3′ of SEQ ID NO: 502:

tacaggtg ccttcttgtc tcaatttgcc ttt

The sequence of the hot spot H4 (5′ to 3′: −151 to −28) corresponds to position 1 to position 124 from 5′ to 3′ of SEQ ID NO: 503:

t tgttttctgc ttttcctgct ctttgtccgc tgatctcctg
ggaagaaagc ttccgaaaag gacaccgttt caggggcgag
tgacgccggg gtgcccaggc cgcgccccag ttccgggttt gca

The sequence of the hot spot HC (5′ to 3′: −845 to −711) corresponds to position 1 to position 135 from 5′ to 3′ of SEQ ID NO: 504:

aggtt ccgaggggcc attgaggaaa ctcctccctt ttaatatcaa
tgtgtattta ttgcaaaaat aatgtagcat cgagtggtat
tttatagctt atccaaaaac ctcctgggtt taacgcattg
tgatagtccc.

### Example 3

### saRNAs Promoted LHPP mRNA Expression and Inhibited Tumor Cell Proliferation

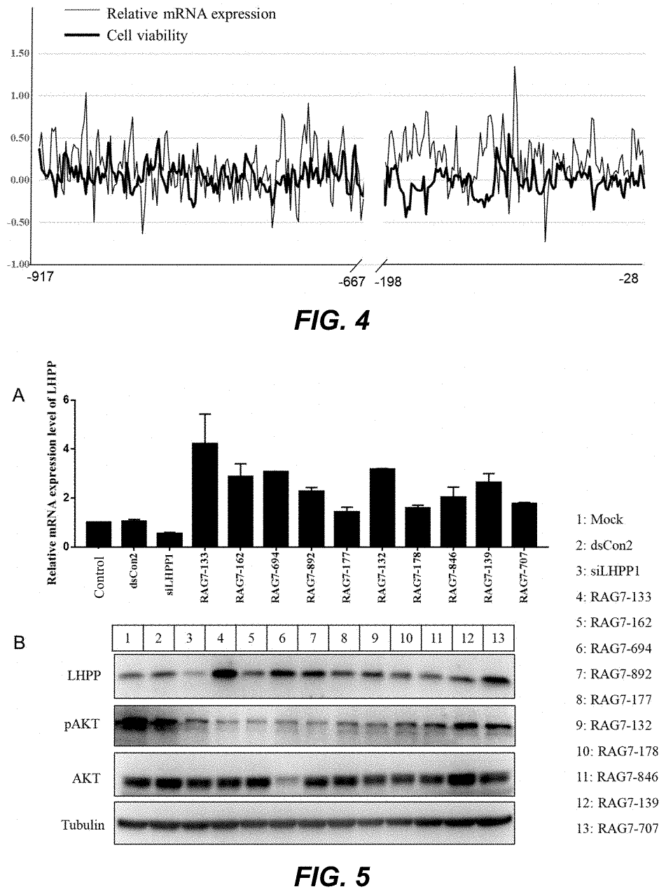

The 290 saRNAs targeting the LHPP promoter were individually transfected into Huh7 cells, and 72 hour later, one-step RT-qPCR was employed to analyze the expression levels of LHPP mRNA and cell viability was detected by the CCK-8 method. As shown in FIG. 4, cell viability decreased when the activating saRNAs promoted the LHPP mRNA expression, and there was a negative correlation between the LHPP mRNA expression level and the cell viability.

The cell viability was detected by the following CCK-8 method: the cCells were plated into a 96-well plate at 3-5×10³ cells/well, cultured overnight, and transfected with the oligonucleotide duplexes. After 72 hour of transfection, 10 uL of CCK-8 solution (Dojindo Molecular Technologies) was added into each well. After 1 hour of incubation at 37° C., a microplate reader was used to measure absorbances at 450 nm.

### Example 4

### saRNAs Promoted LHPP Protein Expression

Cells were plated into a 96-well plate at 3-5×10³ cells/well, cultured overnight, and transfected with 10 randomly selected oligonucleotide duplexes. After 72 h of transfection, the cells were collected and lysed using cell lysis buffer (1×RIPA buffer, CST) containing protease inhibitor. Protein quantification was performed by using the BCA method (Thermo). After polyacrylamide gel electrophoresis separation, then the protein was transferred to a 0.45 μm PVDF membrane. The primary antibody used for the blot assay was a mouse monoclonal anti-LHPP antibody (Invitrogen), a rabbit polyclonal anti-AKT antibody (Cell Signaling Technology), a rabbit polyclonal anti-pAKT antibody (Cell Signaling Technology), or a rabbit polyclonal anti-α/β-tubulin antibody (Cell Signaling Technology); and the secondary antibody used was an anti-mouse IgG HRP-linked antibody (Cell Signaling Technology) or an anti-rabbit IgG HRP-linked antibody (Cell Signaling Technology). Image Lab (BIO-RAD, Chemistry Doctm MP imaging system) was used to scan detecting signals.

TABLE 5
Double-stranded RNA sequences as study controls

| Double-stranded | | |
|---|---|---|
| RNA | Sequence No. | Sequence (5′-3′) |
| dsCon2-sense | SEQ ID NO: 505 | ACUACUGAGUGACAGUAGATT |
| strand | SEQ ID NO: 506 | UCUACUGUCACUCAGUAGUTT |
| dsCon2- | | |
| antisense | | |
| strand | | |
| siLHPP1-sense | SEQ ID NO: 507 | GAAGUUCAGAGCCGCUCAATT |
| strand | SEQ ID NO: 508 | UUGAGCGGCUCUGAACUUCTT |
| siLHPP1- | | |
| antisense | | |
| strand | | |

As shown in FIG. 5, the 10 randomly selected saRNAs downregulated the phosphorylation of AKT, while promoting or increasing the LHPP mRNA and protein expression.

### Example 5

### saRNAs Inhibited Proliferation of A Variety of Tumor Cells

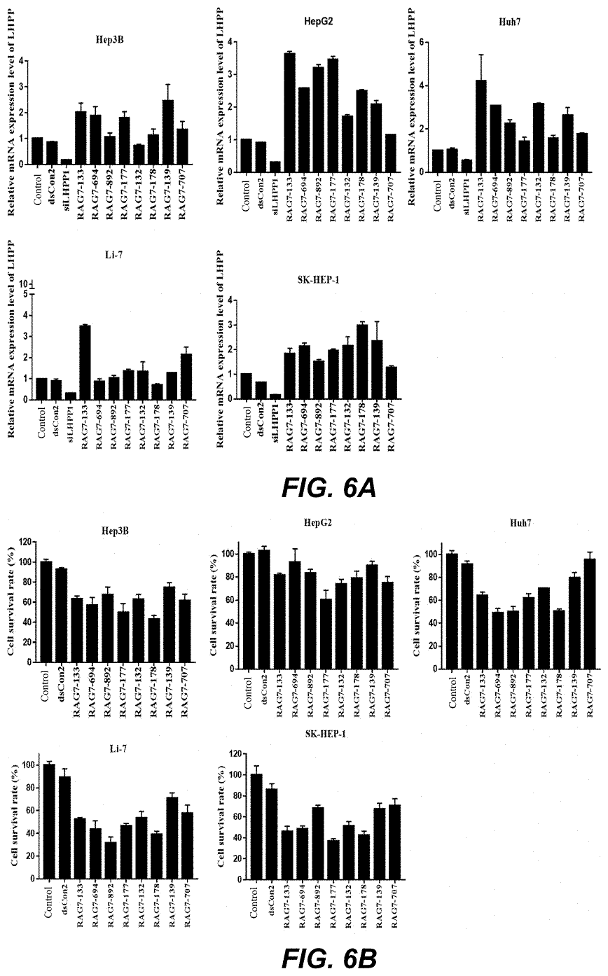

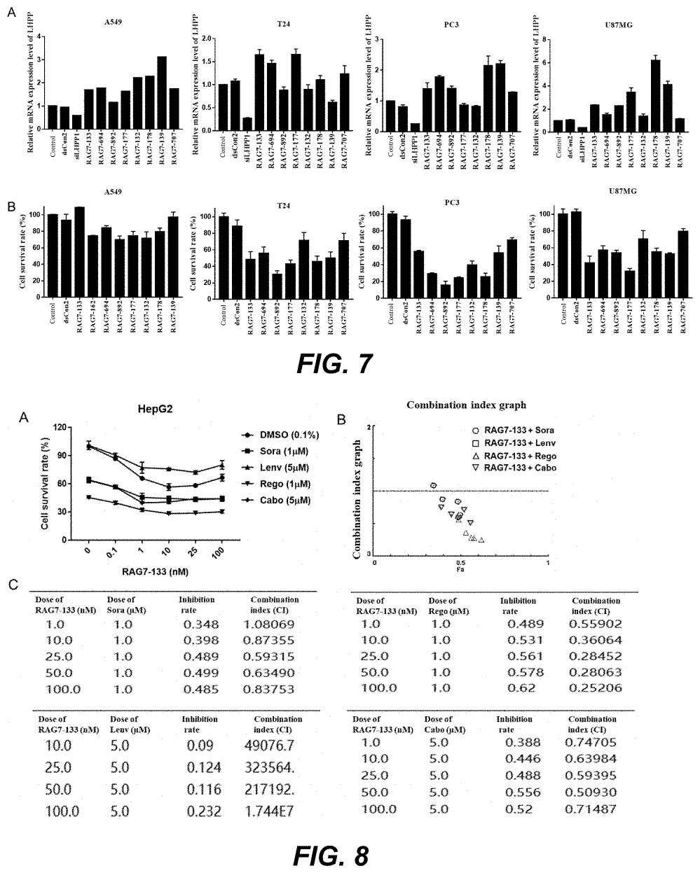

In order to further evaluate the effect of LHPP saRNAs in inducing the mRNA expression of the LHPP gene and inhibiting the proliferation of cancer cells, eight screened saRNAs (RAG7-132, RAG7-133, RAG7-139, RAG7-177, RAG7-178, RAG7-694, RAG7-707 and RAG7-892) each were transfected into the liver cancer cell lines Huh7 (Medical Cell Resource Center, Tohoku University, Japan), HepG2 (ATCC), Hep3B (ATCC), Li-7 (Medical Cell Resource Center, Tohoku University, Japan) and SK-HEP-1 (ATCC), a lung cancer cell line A549 (ATCC), a bladder cancer cell line T24 (ATCC), a prostatic cancer cell line PC3 (ATCC), and a glioma cell line U87MG (ATCC). The mRNA expression and cell viability of the transfected cells were measured. As shown in FIG. 6, RAG7-133 induced the expression of LHPP gene to different degrees and inhibited cell proliferation in all five liver cancer cell lines; and RAG7-694 induced the expression of LHPP gene to different degrees and inhibited cell proliferation in four of the liver cancer cell lines, other than Li-7. In another aspect, all of the aforementioned 8 saRNAs induced the expression of LHPP gene to different degrees and inhibited cell proliferation in the cell lines HepG2 and SK-HEP-1. As shown in FIG. 7, RAG7-133 induced the expression of LHPP gene to different degrees and inhibited cell proliferation in the cell lines T24, PC3, and U87MG; RAG7-694 induced the expression of LHPP gene to different degrees and inhibited cell proliferation in the cell lines A549, T24 and PC3; RAG7-177 induced the expression of LHPP gene to different degrees and inhibited cell proliferation in the cell lines A549, T24, and U87MG; and RAG7-178 induced the expression of LHPP gene to different degrees and inhibited cell proliferation in the cell lines A549, PC3, and U87MG.

### Example 6

### saRNAs in Combination with Chemotherapies Inhibited Cell Proliferation

The compounds used in the study included: Sorafenib (Sora) (SELLECK, S1040), Lenvatinib (Lenv) (SELLECK, S1164), Regorafenib (Rego) (SELLECK, S1178), and Cabozantinib (Cabo) (SELLECK, S1119). Cells were transfected with each candidate saRNAs at varying concentration gradients for 24 hours. Thereafter, the aforementioned compounds were added to the transfected cells at a concentration of 5 μM and the cells were incubated with the compounds for 48 hours. Cell viability was measured using the CCK-8 method. Compusyn© version 1.0 software (ComboSyn, Inc. Paramus, N.J., USA) was used to analyze the combination index (CI) of drugs, wherein CI<1 represented a synergistic effect, CI=1 represented an additive effect, and CI>1 represented an antagonistic effect.

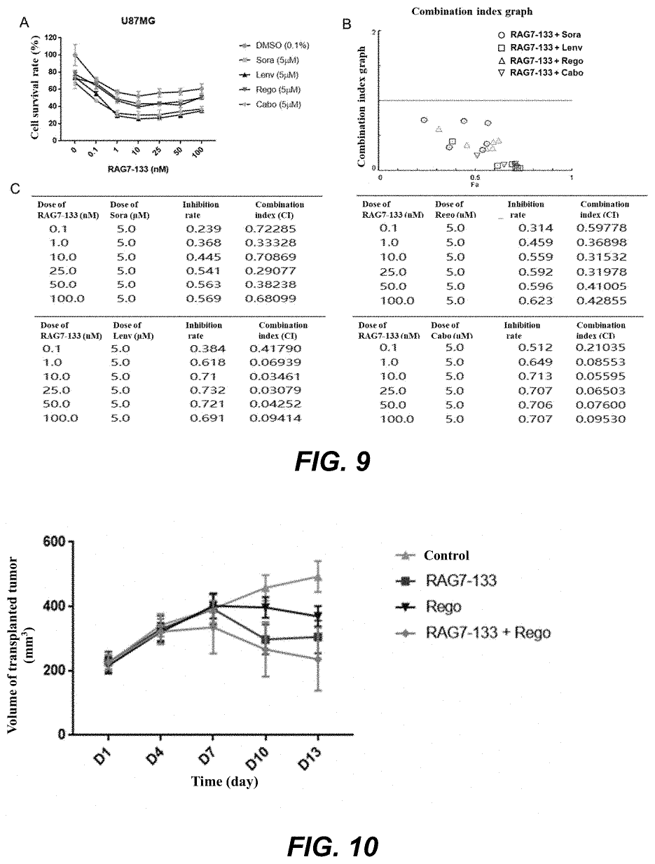

As shown for HepG2 cells in FIG. 8, a high dose (25 nM to 100 nM) of RAG7-133 saRNA and Regorafenib had a strong synergistic effect (CI<0.3); a low dose (1.0 nM to 10 nM) of RAG7-133 and Regorafenib had a synergistic effect (0.3<CI<0.7); and RAG7-133 saRNA had a synergistic effect with Sorafenib and Cabozantinib (CI<1), but did not have a synergistic effect with Lenvatinib (CI>1). As shown for U87MG cells in FIG. 9, RAG7-133 saRNA had a synergistic effect (CI<1) with all the four compounds, in particular, when used in combination with Lenvatinib or Cabozantinib. RAG7-133 saRNA had an extremely strong synergistic effect (CI<0.1) with the compounds within a wide dosage range (1.0 nM to 100 nM).

### Example 7

### Drug Combination Inhibited Tumor Growth In Vivo in Mice Xenograped with Human HepG2

To prepare the saRNA formulation, the in vivo-jetPEI (201-10G, Polyplus-transfection, France) was adopted as an saRNA delivery system. The preparation process is briefly described as follows. An saRNA was first diluted in 10% glucose solution to obtain a solution A. According to the instructions of the manufacturer, a required amount of in vivo-jetPEI was diluted in 10% glucose solution to obtain a solution B. Equal volumes of the solution A and the solution B were mixed (nitrogen-to-phosphorus ratio: 8; final concentration of glucose: 5%). After mixing, the mixture was let to stand at room temperature for 15 minutes for later use.

HepG2 cells in the logarithmic growth phase were obtained and counted, and then the cell suspension was regulated to 5×10⁷ cells/mL and subcutaneously inoculated into the right armpit of BALB/c nude mice at a volume of 0.1 mL per mouse. When tumors in nude mice grew to about 100 mm³, the nude mice were randomly divided into four groups each with six mice: (vehicle control (Vehicle) group, saRNA group, regorafenib group, and saRNA and regorafenib combination group (saRNA+regorafenib)). For the saRNA group and the saRNA+regorafenib group, intratumor injection of saRNA was performed at 1 mg·kg⁻¹ on days 1, 4, 7 and 10. For the regorafenib group and the saRNA+regorafenib group, intragastric administration of regorafenib was performed at 3 mg kg⁻¹ everyday from day 1 through day 12. Starting from the initial administration, the long diameter and the short diameter of each tumor were measured with a vernier caliper every two days. The tumor volume was calculated according to the formula V=(l×w²)/2, wherein 1 represents the longest diameter of the tumor and w represents the diameter parallel to the surface of the tumor and perpendicular to the long diameter. A tumor growth curve and size and morphology of the tumor after anatomy were recorded during the administration. As shown in FIG. 10, compared with the vehicle control group (Vehicle), the tumors began to show the tendency to grow slowly and shrink as of day 7 in the saRNA group (given RAG7-133 alone), and by day 13, the tumor volume increased by 34% compared with that at the beginning of treatment in the saRNA group, while the tumor volume increased by 118% in the vehicle control group. There is a significant difference (P<0.05) between the tumor volume changes of the two groups, indicating that the LHPP saRNA can remarkably inhibit tumor growth in vivo in mice. In the saRNA and regorafenib combination group (RAG7-133+regorafenib), the tumors began to show the tendency to grow slowly as of day 4 and began to shrink on day 7 and by day 13 the tumor volume only increased by 4% compared with that at the beginning of treatment, while the tumor volume in the group given the chemotherapy regorafenib (Rego) alone increased by 70% on day 13 of treatment compared with that at the beginning of treatment. There is a significant difference (P<0.01) between the tumor volume changes of the two groups, indicating that saRNA in combination with the chemotherapy synergistically enhances the cancer inhibition effect of the chemotherapy.

### Example 8

### Drug Combination Inhibited Tumor Growth In Vivo in Mice Xenograped with Human U87MG

To prepare the saRNA formulation, in vivo-jetPEI (201-10G, Polyplus-transfection, France) was adopted as an saRNA delivery system. The preparation process is briefly described as follows: an saRNA was first diluted in a 10% glucose solution to give a solution A; a required amount of in vivo-jetPEI was diluted in 10% glucose solution to give a solution B; then, equal volumes of the solution A and the solution B were mixed (nitrogen-to-phosphorus ratio: 8; final concentration of glucose: 5%). After mixing the mixture was let to stand under room temperature for 15 minutes for later use.

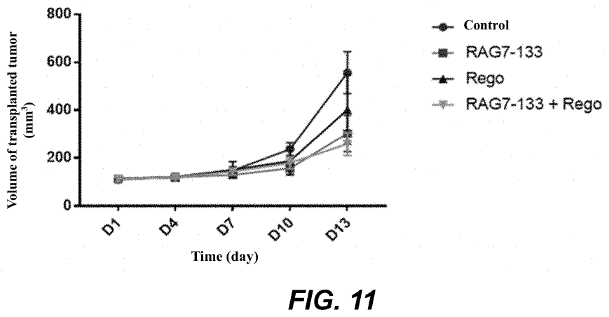

The glioma cell line U87MG were grown to the logarithmic phase, counted, then subcutaneously inoculated at a concentration of 9×10⁷ cells/mL into the right armpit of BALB/c nude mice at 0.1 mL per mouse. Tumor-bearing nude mice were randomly divided into four groups after tumors grew to about 100 mm³ (vehicle control group, saRNA group, regorafenib group, and saRNA and regorafenib in combination group (RAG7-133+regorafenib group)) with seven mice in each group. For the saRNA group and the saRNA+regorafenib group, intratumor injection of saRNA at 1 mg·kg⁻¹ was performed on days 1, 4, 7 and 10. For the regorafenib group and the saRNA+regorafenib group, intragastric administeration of regorafenib at 3 mg·kg⁻¹ was performed every day on day 1 through day 12. After the initial administration, the long diameter and short diameter of each tumor were measured with a vernier caliper every two days. The tumor volume was calculated according to the formula V=(l×w²)/2, wherein 1 represents the longest diameter of the tumor and w represents the diameter parallel to the surface of the tumor and perpendicular to the long diameter. A tumor growth curve and size and morphology of the tumor anatomy were recorded during the administration. As shown in FIG. 11, compared with the vehicle control group (Vehicle), the tumor volume increased by 167% on day 13 when compared with that at the beginning of treatment in the saRNA group (given RAG7-133 alone), while the tumor volume increased by 406% in the control group. There is a significant difference (P<0.05) between the tumor volume changes of the two groups, indicating that the LHPP saRNA can remarkably inhibit tumor growth in vivo in mice. In the saRNA and regorafenib combination group (RAG7-133+regorafenib), the tumor volume increased by 132% on day 13 compared with that at the beginning of treatment, while the tumor volume increased by 251% on day 13 compared with that at the beginning of treatment in the group given regorafenib (Rego) alone. There is a significant difference (P<0.05) between the tumor volume changes of the two groups, indicating that the saRNA in combination with the chemotherapy synergistically enhances the cancer inhibition effect of the chemotherapy.

Based on the results above, a plurality of saRNAs capable of remarkably activating the expression of LHPP gene were identified through high-throughput screening of saRNAs targeting LHPP gene promoter. These saRNAs inhibit the proliferation of tumor cells in vitro or in vivo by up-regulating the expression of LHPP gene and protein and downregulating the phosphorylation of AKT. These results clearly suggest that saRNAs targeting the LHPP gene promoter can be a promising strategy for tumor treatment.

### REFERENCES

1. Yokoi F, Hiraishi H, Izuhara K. 2003. Molecular cloning of a cDNA for the human phospholysine phosphohistidine inorganic pyrophosphate phosphatase. J Biochem 133:607-14.
2. Neff C D, Abkevich V, Packer J C, Chen Y, Potter J, et al. 2009. Evidence for HTR1A and LHPP as interacting genetic risk factors in major depression. Mol Psychiatry 14:621-30.
3. Gohla A. 2019. Do metabolic HAD phosphatases moonlight as protein phosphatases? Biochim Biophys Acta Mol Cell Res 1866:153-66.
4. CONVERGE consortium. 2015. Sparse whole-genome sequencing identifies two loci for major depressive disorder. Nature 523:588-91.
5. Knowles E E, Kent J W, Jr., McKay D R, Sprooten E, Mathias S R, et al. 2016. Genome-wide linkage on chromosome 10q26 for a dimensional scale of major depression. J Affect Disord 191:123-31.
6. Polimanti R, Wang Q, Meda S A, Patel K T, Pearlson G D, et al. 2017. The Interplay Between Risky Sexual Behaviors and Alcohol Dependence: Genome-Wide Association and Neuroimaging Support for LHPP as a Risk Gene. Neuropsychopharmacology 42:598-605.
7. Cui L, Gong X, Tang Y, Kong L, Chang M, et al. 2016. Relationship between the LHPP Gene Polymorphism and Resting-State Brain Activity in Major Depressive Disorder. Neural Plast 2016:9162590.
8. Lesseur C, Diergaarde B, Olshan A F, Wunsch-Filho V, Ness A R, et al. 2016. Genome-wide association analyses identify new susceptibility loci for oral cavity and pharyngeal cancer. Nat Genet 48:1544-50.
9. Gutierrez-Camino A, Martin-Guerrero I, Garcia-Orad A. 2017. Genetic susceptibility in childhood acute lymphoblastic leukemia. Med Oncol 34:179.
10. Vijayakrishnan J, Kumar R, Henrion M Y, Moorman A V, Rachakonda P S, et al. 2017. A genome-wide association study identifies risk loci for childhood acute lymphoblastic leukemia at 10q26.13 and 12q23.1. Leukemia 31:573-9.
11. Hindupur S K, Colombi M, Fuhs S R, Matter M S, Guri Y, et al. 2018. The protein histidine phosphatase LHPP is a tumour suppressor. Nature 555:678-82.
12. Bray F, Ferlay J, Soerjomataram I, Siegel R L, Torre L A, Jemal A. 2018. Global cancer statistics 2018: GLOBOCAN estimates of incidence and mortality worldwide for 36 cancers in 185 countries. CA Cancer J Clin 68:394-424.

1. A small activating RNA (saRNA) comprising a sense nucleic acid fragment and an antisense nucleic acid fragment, the sense nucleic acid fragment having at least 90% homology or complementarity to a continuous sequence of 16 to 35 nucleotides in length of any one of SEQ ID NOs: 500.

2. The saRNA of claim 1, wherein the sense nucleic acid fragment and the antisense nucleic acid fragment are located on two different nucleic acid strands or on an identical nucleic acid strand, preferably forming a hairpin single-stranded nucleic acid molecule, wherein the sense nucleic acid fragment and the antisense nucleic acid fragment comprise complementary regions, wherein the complementary regions form a double-stranded nucleic acid structure between the two fragments that can activate the expression of LHPP in a cell.

3. The saRNA of claim 1, wherein the sense nucleic acid fragment and the antisense nucleic acid fragment are located on an identical nucleic acid strand, forming a hairpin single-stranded nucleic acid molecule.

4. The saRNA of claim 2, wherein at least one nucleic acid fragment has 3′ overhang of 0 to 6 nucleotides in length.

5. The saRNA of claim 4, wherein the sense nucleic acid fragment and the antisense nucleic acid fragment has a 3′ overhang of 2 or 3 nucleotides in length.

6. The saRNA of claim 5, wherein the sense nucleic acid fragment and the antisense nucleic acid fragment independently are 16 to 35 nucleotides.

7. The saRNA of claim 1, wherein one fragment of the saRNA having at least 90% homology or complementarity to nucleotide sequence selected from the group consisting of SEQ ID NOs:329-492.

8. The saRNA of claim 7, wherein the sense nucleic acid fragment of the saRNA having at least 90% homology to nucleotide sequence selected from the group consisting of SEQ ID NOs:1-164, and an antisense nucleic acid fragment having at least 90% homology to nucleotide sequence selected from the group consisting of SEQ ID NOs:165-328.

9. The saRNA of claim 8, wherein the sense nucleic acid fragment of the saRNA comprises nucleotide sequence selected from the group consisting of SEQ ID NOs:1-164, and the antisense nucleic acid fragment comprises any nucleotide sequence chosen from SEQ ID NOs: 165-328.

10. The saRNA of claim 1, wherein the saRNA comprises:
i. at least one chemically modified nucleotide, or
ii. one or more modifications selected from the group consisting of:
a. modification of a phosphodiester bond connecting nucleotides in the nucleotide sequence of the saRNA;
b. modification of 2′-OH of a ribose in the nucleotide sequence of the saRNA;
c. modification of a base in the nucleotide sequence of the saRNA;
d. at least one nucleotide in the nucleotide sequence of the saRNA being a locked nucleic acid.

11. The saRNA of claim 1, wherein the nucleotides in the saRNA are not chemically modified nucleotides.

12. (canceled)

13. The saRNA of claim 1, wherein the saRNA activating nucleic acid molecule activates or upregulates the expression of LHPP by at least 10%.

14. A nucleic acid encoding the saRNA of claim 1, wherein the nucleic acid is a DNA or RNA molecule.

15. (canceled)

16. (canceled)

17. A composition comprising the saRNA of claim 1, and, a pharmaceutically acceptable carrier, wherein the pharmaceutically acceptable carrier is an aqueous carrier, a liposome, a high molecular polymer, or a polypeptide.

18. (canceled)

19. The composition of claim 17, wherein the composition comprises 1-150 nM of the saRNA.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method for activating or upregulating the expression of LHPP in a cell, comprising administering a composition of claim 1 to the cell.

32. (canceled)

33. (canceled)

34. (canceled)

35. (canceled)

36. (canceled)

37. (canceled)

38. (canceled)

39. A method for treating a disease or condition related to insufficient or decreased expression of LHPP protein in a human patient in need thereof, comprising administering a composition of claim 1.

40. The method of claim 39, wherein the disease or condition related to insufficient or decreased expression of LHPP protein comprises solid tumors, wherein the solid tumor is selected from the group consisting of liver cancer, lung cancer, bladder cancer, prostatic cancer, and glioma.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. The saRNA of claim 2, wherein the sense nucleic acid fragment and the antisense nucleic acid fragment has a 3′ overhang of 2 or 3 nucleotides in length, and wherein the nucleotide of the overhang is dT.

54. The saRNA of claim 1, containing a sense nucleic acid fragment and an antisense nucleic acid fragment combination selected from the group consisting of:
SEQ ID NO:1 and SEQ ID NO:165;
SEQ ID NO:2 and SEQ ID NO:166;
SEQ ID NO:3 and SEQ ID NO:167;
SEQ ID NO:4 and SEQ ID NO:168;
SEQ ID NO:5 and SEQ ID NO:169;
SEQ ID NO:6 and SEQ ID NO:170;
SEQ ID NO:7 and SEQ ID NO:171;
SEQ ID NO:8 and SEQ ID NO:172;
SEQ ID NO:9 and SEQ ID NO:173;
SEQ ID NO:10 and SEQ ID NO:174;
SEQ ID NO:11 and SEQ ID NO:175;
SEQ ID NO:12 and SEQ ID NO:176;
SEQ ID NO:13 and SEQ ID NO:177;
SEQ ID NO:14 and SEQ ID NO:178;
SEQ ID NO:15 and SEQ ID NO:179;
SEQ ID NO:16 and SEQ ID NO:180;
SEQ ID NO:17 and SEQ ID NO:181;
SEQ ID NO:18 and SEQ ID NO:182;
SEQ ID NO:19 and SEQ ID NO:183;
SEQ ID NO:20 and SEQ ID NO:184;
SEQ ID NO:21 and SEQ ID NO:185;
SEQ ID NO:22 and SEQ ID NO:186;
SEQ ID NO:23 and SEQ ID NO:187;
SEQ ID NO:24 and SEQ ID NO:188;
SEQ ID NO:25 and SEQ ID NO:189;
SEQ ID NO:26 and SEQ ID NO:190;
SEQ ID NO:27 and SEQ ID NO:191;
SEQ ID NO:28 and SEQ ID NO:192;
SEQ ID NO:29 and SEQ ID NO:193;
SEQ ID NO:30 and SEQ ID NO:194;
SEQ ID NO:31 and SEQ ID NO:195;
SEQ ID NO:32 and SEQ ID NO:196;
SEQ ID NO:33 and SEQ ID NO:197;
SEQ ID NO:34 and SEQ ID NO:198;
SEQ ID NO:35 and SEQ ID NO:199;
SEQ ID NO:36 and SEQ ID NO:200;
SEQ ID NO:37 and SEQ ID NO:201;
SEQ ID NO:38 and SEQ ID NO:202;
SEQ ID NO:39 and SEQ ID NO:203;
SEQ ID NO:40 and SEQ ID NO:204;
SEQ ID NO:41 and SEQ ID NO:205;
SEQ ID NO:42 and SEQ ID NO:206;
SEQ ID NO:43 and SEQ ID NO:207;
SEQ ID NO:44 and SEQ ID NO:208;
SEQ ID NO:45 and SEQ ID NO:209;
SEQ ID NO:46 and SEQ ID NO:210;
SEQ ID NO:47 and SEQ ID NO:211;
SEQ ID NO:48 and SEQ ID NO:212;
SEQ ID NO:49 and SEQ ID NO:213;
SEQ ID NO:50 and SEQ ID NO:214;
SEQ ID NO:51 and SEQ ID NO:215;
SEQ ID NO:52 and SEQ ID NO:216;
SEQ ID NO:53 and SEQ ID NO:217;
SEQ ID NO:54 and SEQ ID NO:218;
SEQ ID NO:55 and SEQ ID NO:219;
SEQ ID NO:56 and SEQ ID NO:220;
SEQ ID NO:57 and SEQ ID NO:221;
SEQ ID NO:58 and SEQ ID NO:222;
SEQ ID NO:59 and SEQ ID NO:223;
SEQ ID NO:60 and SEQ ID NO:224;
SEQ ID NO:61 and SEQ ID NO:225;
SEQ ID NO:62 and SEQ ID NO:226;
SEQ ID NO:63 and SEQ ID NO:227;
SEQ ID NO:64 and SEQ ID NO:228;
SEQ ID NO:65 and SEQ ID NO:229;
SEQ ID NO:66 and SEQ ID NO:230;
SEQ ID NO:67 and SEQ ID NO:231;
SEQ ID NO:68 and SEQ ID NO:232;
SEQ ID NO:69 and SEQ ID NO:233;
SEQ ID NO:70 and SEQ ID NO:234;
SEQ ID NO:71 and SEQ ID NO:235;
SEQ ID NO:72 and SEQ ID NO:236;
SEQ ID NO:73 and SEQ ID NO:237;
SEQ ID NO:74 and SEQ ID NO:238;
SEQ ID NO:75 and SEQ ID NO:239;
SEQ ID NO:76 and SEQ ID NO:240;
SEQ ID NO:77 and SEQ ID NO:241;
SEQ ID NO:78 and SEQ ID NO:242;
SEQ ID NO:79 and SEQ ID NO:243;
SEQ ID NO:80 and SEQ ID NO:244;
SEQ ID NO:81 and SEQ ID NO:245;
SEQ ID NO:82 and SEQ ID NO:246;
SEQ ID NO:83 and SEQ ID NO:247;
SEQ ID NO:84 and SEQ ID NO:248;
SEQ ID NO:85 and SEQ ID NO:249;
SEQ ID NO:86 and SEQ ID NO:250;
SEQ ID NO:87 and SEQ ID NO:251;
SEQ ID NO:88 and SEQ ID NO:252;
SEQ ID NO:89 and SEQ ID NO:253;
SEQ ID NO:90 and SEQ ID NO:254;
SEQ ID NO:91 and SEQ ID NO:255;
SEQ ID NO:92 and SEQ ID NO:256;
SEQ ID NO:93 and SEQ ID NO:257;
SEQ ID NO:94 and SEQ ID NO:258;
SEQ ID NO:95 and SEQ ID NO:259;
SEQ ID NO:96 and SEQ ID NO:260;
SEQ ID NO:97 and SEQ ID NO:261;
SEQ ID NO:98 and SEQ ID NO:262;
SEQ ID NO:99 and SEQ ID NO:263;
SEQ ID NO:100 and SEQ ID NO:264;
SEQ ID NO:101 and SEQ ID NO:265;
SEQ ID NO:102 and SEQ ID NO:266;
SEQ ID NO:103 and SEQ ID NO:267;
SEQ ID NO:104 and SEQ ID NO:268;
SEQ ID NO:105 and SEQ ID NO:269;
SEQ ID NO:106 and SEQ ID NO:270;
SEQ ID NO:107 and SEQ ID NO:271;
SEQ ID NO:108 and SEQ ID NO:272;
SEQ ID NO:109 and SEQ ID NO:273;
SEQ ID NO:110 and SEQ ID NO:274;
SEQ ID NO:111 and SEQ ID NO:275;
SEQ ID NO:112 and SEQ ID NO:276;
SEQ ID NO:113 and SEQ ID NO:277;
SEQ ID NO:114 and SEQ ID NO:278;
SEQ ID NO:115 and SEQ ID NO:279;
SEQ ID NO:116 and SEQ ID NO:280;
SEQ ID NO:117 and SEQ ID NO:281;
SEQ ID NO:118 and SEQ ID NO:282;
SEQ ID NO:119 and SEQ ID NO:283;
SEQ ID NO:120 and SEQ ID NO:284;
SEQ ID NO:121 and SEQ ID NO:285;
SEQ ID NO:122 and SEQ ID NO:286;
SEQ ID NO:123 and SEQ ID NO:287;
SEQ ID NO:124 and SEQ ID NO:288;
SEQ ID NO:125 and SEQ ID NO:289;
SEQ ID NO:126 and SEQ ID NO:290;
SEQ ID NO:127 and SEQ ID NO:291;
SEQ ID NO:128 and SEQ ID NO:292;
SEQ ID NO:129 and SEQ ID NO:293;
SEQ ID NO:130 and SEQ ID NO:294;
SEQ ID NO:131 and SEQ ID NO:295;
SEQ ID NO:132 and SEQ ID NO:296;
SEQ ID NO:133 and SEQ ID NO:297;
SEQ ID NO:134 and SEQ ID NO:298;
SEQ ID NO:135 and SEQ ID NO:299;
SEQ ID NO:136 and SEQ ID NO:300;
SEQ ID NO:137 and SEQ ID NO:301;
SEQ ID NO:138 and SEQ ID NO:302;
SEQ ID NO:139 and SEQ ID NO:303;
SEQ ID NO:140 and SEQ ID NO:304;
SEQ ID NO:141 and SEQ ID NO:305;
SEQ ID NO:142 and SEQ ID NO:306;
SEQ ID NO:143 and SEQ ID NO:307;
SEQ ID NO:144 and SEQ ID NO:308;
SEQ ID NO:145 and SEQ ID NO:309;
SEQ ID NO:146 and SEQ ID NO:310;
SEQ ID NO:147 and SEQ ID NO:311;
SEQ ID NO:148 and SEQ ID NO:312;
SEQ ID NO:149 and SEQ ID NO:313;
SEQ ID NO:150 and SEQ ID NO:314;
SEQ ID NO:151 and SEQ ID NO:315;
SEQ ID NO:152 and SEQ ID NO:316;
SEQ ID NO:153 and SEQ ID NO:317;
SEQ ID NO:154 and SEQ ID NO:318;
SEQ ID NO:155 and SEQ ID NO:319;
SEQ ID NO:156 and SEQ ID NO:320;
SEQ ID NO:157 and SEQ ID NO:321;
SEQ ID NO:158 and SEQ ID NO:322;
SEQ ID NO:159 and SEQ ID NO:323;
SEQ ID NO:160 and SEQ ID NO:324;
SEQ ID NO:161 and SEQ ID NO:325;
SEQ ID NO:162 and SEQ ID NO:326;
SEQ ID NO:163 and SEQ ID NO:327; and
SEQ ID NO:164 and SEQ ID NO:328.
